SEQUENCE LISTING (229 sequences)

SEQ ID NO: 1 - protein, 8 aa, Artificial Sequence
  Synthetic
LSGRSDNH

SEQ ID NO: 2 - protein, 8 aa, Artificial Sequence
  Synthetic
TGRGPSWV

SEQ ID NO: 3 - protein, 8 aa, Artificial Sequence
  Synthetic
PLTGRSGG

SEQ ID NO: 4 - protein, 8 aa, Artificial Sequence
  Synthetic
TARGPSFK

SEQ ID NO: 5 - protein, 12 aa, Artificial Sequence
  Synthetic
NTLSGRSENHSG

SEQ ID NO: 6 - protein, 12 aa, Artificial Sequence
  Synthetic
NTLSGRSGNHGS

SEQ ID NO: 7 - protein, 12 aa, Artificial Sequence
  Synthetic
TSTSGRSANPRG

SEQ ID NO: 8 - protein, 8 aa, Artificial Sequence
  Synthetic
TSGRSANP

SEQ ID NO: 9 - protein, 10 aa, Artificial Sequence
  Synthetic
VHMPLGFLGP

SEQ ID NO: 10 - protein, 8 aa, Artificial Sequence
  Synthetic
AVGLLAPP

SEQ ID NO: 11 - protein, 8 aa, Artificial Sequence
  Synthetic
AQNLLGMV

SEQ ID NO: 12 - protein, 8 aa, Artificial Sequence
  Synthetic
QNQALRMA

SEQ ID NO: 13 - protein, 8 aa, Artificial Sequence
  Synthetic
LAAPLGLL

SEQ ID NO: 14 - protein, 8 aa, Artificial Sequence
  Synthetic
STFPFGMF

SEQ ID NO: 15 - protein, 8 aa, Artificial Sequence
  Synthetic
ISSGLLSS

SEQ ID NO: 16 - protein, 8 aa, Artificial Sequence
  Synthetic
PAGLWLDP

SEQ ID NO: 17 - protein, 8 aa, Artificial Sequence
  Synthetic
VAGRSMRP

SEQ ID NO: 18 - protein, 8 aa, Artificial Sequence
  Synthetic
VVPEGRRS

SEQ ID NO: 19 - protein, 8 aa, Artificial Sequence
  Synthetic
ILPRSPAF

SEQ ID NO: 20 - protein, 8 aa, Artificial Sequence
  Synthetic
MVLGRSLL

SEQ ID NO: 21 - protein, 8 aa, Artificial Sequence
  Synthetic
QGRAITFI

SEQ ID NO: 22 - protein, 8 aa, Artificial Sequence
  Synthetic
SPRSIMLA

SEQ ID NO: 23 - protein, 8 aa, Artificial Sequence
  Synthetic
SMLRSMPL

SEQ ID NO: 24 - protein, 13 aa, Artificial Sequence
  Synthetic
ISSGLLSGRSDNH

SEQ ID NO: 25 - protein, 17 aa, Artificial Sequence
  Synthetic
AVGLLAPPGGLSGRSDN

SEQ ID NO: 26 - protein, 22 aa, Artificial Sequence
  Synthetic
ISSGLLSSGGSGGSLSGRSDNH

SEQ ID NO: 27 - protein, 8 aa, Artificial Sequence
  Synthetic
LSGRSGNH

SEQ ID NO: 28 - protein, 9 aa, Artificial Sequence
  Synthetic
SGRSANPRG

SEQ ID NO: 29 - protein, 8 aa, Artificial Sequence
  Synthetic
LSGRSDDH

SEQ ID NO: 30 - protein, 8 aa, Artificial Sequence
  Synthetic
LSGRSDIH

SEQ ID NO: 31 - protein, 8 aa, Artificial Sequence
  Synthetic
LSGRSDQH

SEQ ID NO: 32 - protein, 8 aa, Artificial Sequence
  Synthetic
LSGRSDTH

SEQ ID NO: 33 - protein, 8 aa, Artificial Sequence
  Synthetic
LSGRSDYH

SEQ ID NO: 34 - protein, 8 aa, Artificial Sequence
  Synthetic
LSGRSDNP

SEQ ID NO: 35 - protein, 8 aa, Artificial Sequence
  Synthetic
LSGRSANP

SEQ ID NO: 36 - protein, 8 aa, Artificial Sequence
  Synthetic
LSGRSANI

SEQ ID NO: 37 - protein, 8 aa, Artificial Sequence
  Synthetic
LSGRSDNI

SEQ ID NO: 38 - protein, 8 aa, Artificial Sequence
  Synthetic
MIAPVAYR

SEQ ID NO: 39 - protein, 8 aa, Artificial Sequence
  Synthetic
RPSPMWAY

SEQ ID NO: 40 - protein, 8 aa, Artificial Sequence
  Synthetic
WATPRPMR

SEQ ID NO: 41 - protein, 8 aa, Artificial Sequence
  Synthetic
FRLLDWQW

SEQ ID NO: 42 - protein, 5 aa, Artificial Sequence
  Synthetic
ISSGL

SEQ ID NO: 43 - protein, 7 aa, Artificial Sequence
  Synthetic
ISSGLLS

SEQ ID NO: 44 - protein, 6 aa, Artificial Sequence
  Synthetic
ISSGLL

SEQ ID NO: 45 - protein, 15 aa, Artificial Sequence
  Synthetic
ISSGLLSGRSANPRG

SEQ ID NO: 46 - protein, 18 aa, Artificial Sequence
  Synthetic
AVGLLAPPTSGRSANPRG

SEQ ID NO: 47 - protein, 17 aa, Artificial Sequence
  Synthetic
AVGLLAPPSGRSANPRG

SEQ ID NO: 48 - protein, 13 aa, Artificial Sequence
  Synthetic
ISSGLLSGRSDDH

SEQ ID NO: 49 - protein, 13 aa, Artificial Sequence
  Synthetic
ISSGLLSGRSDIH

SEQ ID NO: 50 - protein, 13 aa, Artificial Sequence
  Synthetic
ISSGLLSGRSDQH

SEQ ID NO: 51 - protein, 13 aa, Artificial Sequence
  Synthetic
ISSGLLSGRSDTH

SEQ ID NO: 52 - protein, 13 aa, Artificial Sequence
  Synthetic
ISSGLLSGRSDYH

SEQ ID NO: 53 - protein, 13 aa, Artificial Sequence
  Synthetic
ISSGLLSGRSDNP

SEQ ID NO: 54 - protein, 13 aa, Artificial Sequence
  Synthetic
ISSGLLSGRSANP

SEQ ID NO: 55 - protein, 13 aa, Artificial Sequence
  Synthetic
ISSGLLSGRSANI

SEQ ID NO: 56 - protein, 18 aa, Artificial Sequence
  Synthetic
AVGLLAPPGGLSGRSDDH

SEQ ID NO: 57 - protein, 18 aa, Artificial Sequence
  Synthetic
AVGLLAPPGGLSGRSDIH

SEQ ID NO: 58 - protein, 18 aa, Artificial Sequence
  Synthetic
AVGLLAPPGGLSGRSDQH

SEQ ID NO: 59 - protein, 18 aa, Artificial Sequence
  Synthetic
AVGLLAPPGGLSGRSDTH

SEQ ID NO: 60 - protein, 18 aa, Artificial Sequence
  Synthetic
AVGLLAPPGGLSGRSDYH

SEQ ID NO: 61 - protein, 18 aa, Artificial Sequence
  Synthetic
AVGLLAPPGGLSGRSDNP

SEQ ID NO: 62 - protein, 18 aa, Artificial Sequence
  Synthetic
AVGLLAPPGGLSGRSANP

SEQ ID NO: 63 - protein, 18 aa, Artificial Sequence
  Synthetic
AVGLLAPPGGLSGRSANI

SEQ ID NO: 64 - protein, 13 aa, Artificial Sequence
  Synthetic
ISSGLLSGRSDNI

SEQ ID NO: 65 - protein, 18 aa, Artificial Sequence
  Synthetic
AVGLLAPPGGLSGRSDNI

SEQ ID NO: 66 - protein, 19 aa, Artificial Sequence
  Synthetic
GLSGRSDNHGGAVGLLAPP

SEQ ID NO: 67 - protein, 21 aa, Artificial Sequence
  Synthetic
GLSGRSDNHGGVHMPLGFLGP

SEQ ID NO: 68 - protein, 5 aa, Artificial Sequence
  Synthetic
GSGGS

SEQ ID NO: 69 - protein, 4 aa, Artificial Sequence
  Synthetic
GGGS

SEQ ID NO: 70 - protein, 4 aa, Artificial Sequence
  Synthetic
GGSG

SEQ ID NO: 71 - protein, 5 aa, Artificial Sequence
  Synthetic
GGSGG

SEQ ID NO: 72 - protein, 5 aa, Artificial Sequence
  Synthetic
GSGSG

SEQ ID NO: 73 - protein, 5 aa, Artificial Sequence
  Synthetic
GSGGG

SEQ ID NO: 74 - protein, 5 aa, Artificial Sequence
  Synthetic
GGGSG

SEQ ID NO: 75 - protein, 5 aa, Artificial Sequence
  Synthetic
GSSSG

SEQ ID NO: 76 - protein, 13 aa, Artificial Sequence
  Synthetic
GSSGGSGGSGGSG

SEQ ID NO: 77 - protein, 11 aa, Artificial Sequence
  Synthetic
GSSGGSGGSGG

SEQ ID NO: 78 - protein, 12 aa, Artificial Sequence
  Synthetic
GSSGGSGGSGGS

SEQ ID NO: 79 - protein, 16 aa, Artificial Sequence
  Synthetic
GSSGGSGGSGGSGGGS

SEQ ID NO: 80 - protein, 10 aa, Artificial Sequence
  Synthetic
GSSGGSGGSG

SEQ ID NO: 81 - protein, 11 aa, Artificial Sequence
  Synthetic
GSSGGSGGSGS

SEQ ID NO: 82 - protein, 4 aa, Artificial Sequence
  Synthetic
GGGS

SEQ ID NO: 83 - protein, 5 aa, Artificial Sequence
  Synthetic
GSSGT

SEQ ID NO: 84 - protein, 4 aa, Artificial Sequence
  Synthetic
GSSG

SEQ ID NO: 85 - protein, 15 aa, Artificial Sequence
  Synthetic
YLCQRHPLALKYCTN

SEQ ID NO: 86 - protein, 15 aa, Artificial Sequence
  Synthetic
PLCVPTQLLRSCYNY

SEQ ID NO: 87 - protein, 15 aa, Artificial Sequence
  Synthetic
AVCHPLANVETQCLD

SEQ ID NO: 88 - protein, 15 aa, Artificial Sequence
  Synthetic
PHCHPLFNNTYCYRH

SEQ ID NO: 89 - protein, 15 aa, Artificial Sequence
  Synthetic
PLCRPIELLASCPMK

SEQ ID NO: 90 - protein, 15 aa, Artificial Sequence
  Synthetic
GAACVSAWGFFCECC

SEQ ID NO: 91 - protein, 15 aa, Artificial Sequence
  Synthetic
DCAKDILHLMPHCSM

SEQ ID NO: 92 - protein, 15 aa, Artificial Sequence
  Synthetic
NTCMHPLLLQGCKTY

SEQ ID NO: 93 - protein, 15 aa, Artificial Sequence
  Synthetic
YLGCLLYAGPGCEGG

SEQ ID NO: 94 - protein, 15 aa, Artificial Sequence
  Synthetic
ARCPHPLLLSICENN

SEQ ID NO: 95 - protein, 15 aa, Artificial Sequence
  Synthetic
ELCPHPLPFGFCNNY

SEQ ID NO: 96 - protein, 15 aa, Artificial Sequence
  Synthetic
ALYCHPPYIRCEEMT

SEQ ID NO: 97 - protein, 15 aa, Artificial Sequence
  Synthetic
TSLCHPVMIMYCKTG

SEQ ID NO: 98 - protein, 15 aa, Artificial Sequence
  Synthetic
PLCHPLEQASWCNMD

SEQ ID NO: 99 - protein, 15 aa, Artificial Sequence
  Synthetic
PHPCPRTGSRMCHFS

SEQ ID NO: 100 - protein, 15 aa, Artificial Sequence
  Synthetic
SGCRHPLPLKACGTN

SEQ ID NO: 101 - protein, 15 aa, Artificial Sequence
  Synthetic
GLCHPIRLHNTQCTI

SEQ ID NO: 102 - protein, 15 aa, Artificial Sequence
  Synthetic
KCMHPLNLHNINCNH

SEQ ID NO: 103 - protein, 15 aa, Artificial Sequence
  Synthetic
PICHPLREFMNTCFK

SEQ ID NO: 104 - protein, 15 aa, Artificial Sequence
  Synthetic
NCHPLDVVGWLGCMK

SEQ ID NO: 105 - protein, 15 aa, Artificial Sequence
  Synthetic
YNNVCHPLFCSQHTY

SEQ ID NO: 106 - protein, 15 aa, Artificial Sequence
  Synthetic
TFCHPLFSLNYCGHK

SEQ ID NO: 107 - protein, 15 aa, Artificial Sequence
  Synthetic
FCHPLTLSNNKQCNR

SEQ ID NO: 108 - protein, 15 aa, Artificial Sequence
  Synthetic
LSHCAVLLLRVCSGS

SEQ ID NO: 109 - protein, 15 aa, Artificial Sequence
  Synthetic
KIHCHPLRLGTCLVG

SEQ ID NO: 110 - protein, 15 aa, Artificial Sequence
  Synthetic
ETCAHPLDMRMCRHN

SEQ ID NO: 111 - protein, 15 aa, Artificial Sequence
  Synthetic
PLCYPLILMSSCWLG

SEQ ID NO: 112 - protein, 15 aa, Artificial Sequence
  Synthetic
YGICHPAPDLPCMQI

SEQ ID NO: 113 - protein, 15 aa, Artificial Sequence
  Synthetic
TACHPLYNVEHLCEI

SEQ ID NO: 114 - protein, 15 aa, Artificial Sequence
  Synthetic
TACNKSVCVAGCCLL

SEQ ID NO: 115 - protein, 15 aa, Artificial Sequence
  Synthetic
LHPLCSYMKSCMKNN

SEQ ID NO: 116 - protein, 15 aa, Artificial Sequence
  Synthetic
THCHCMVYFCPCRWS

SEQ ID NO: 117 - protein, 15 aa, Artificial Sequence
  Synthetic
PKCPHPLHLANCYAS

SEQ ID NO: 118 - protein, 15 aa, Artificial Sequence
  Synthetic
  MISC_FEATURE (12)..(12): Xaa = any amino acid
KTCYHPTPVIAXNSY

SEQ ID NO: 119 - protein, 15 aa, Artificial Sequence
  Synthetic
AKCLPPLIQYCRCIK

SEQ ID NO: 120 - protein, 15 aa, Artificial Sequence
  Synthetic
HACQHPLQLHTCKHN

SEQ ID NO: 121 - protein, 15 aa, Artificial Sequence
  Synthetic
LCHPLVLSAWESCSN

SEQ ID NO: 122 - protein, 15 aa, Artificial Sequence
  Synthetic
WPLCSFGKSFCAQNA

SEQ ID NO: 123 - protein, 15 aa, Artificial Sequence
  Synthetic
ECQSFEHFLTNNCHS

SEQ ID NO: 124 - protein, 15 aa, Artificial Sequence
  Synthetic
SCKHPLVMPNLKCTR

SEQ ID NO: 125 - protein, 15 aa, Artificial Sequence
  Synthetic
YPCHPLQLSIPHCTK

SEQ ID NO: 126 - protein, 15 aa, Artificial Sequence
  Synthetic
ICHPLTHTMEYMCMN

SEQ ID NO: 127 - protein, 15 aa, Artificial Sequence
  Synthetic
TLCHPLTFSVPTCTN

SEQ ID NO: 128 - protein, 15 aa, Artificial Sequence
  Synthetic
PLCQPNRLLQACGNT

SEQ ID NO: 129 - protein, 15 aa, Artificial Sequence
  Synthetic
TLCRHPLALDGCQNN

SEQ ID NO: 130 - protein, 15 aa, Artificial Sequence
  Synthetic
QPMCYQPAHPLCNTI

SEQ ID NO: 131 - protein, 15 aa, Artificial Sequence
  Synthetic
SNCHPLLFQHYHCML

SEQ ID NO: 132 - protein, 15 aa, Artificial Sequence
  Synthetic
EKCYHPLTLAHCQNH

SEQ ID NO: 133 - protein, 15 aa, Artificial Sequence
  Synthetic
NKCFVHPLAMPNCNS

SEQ ID NO: 134 - protein, 15 aa, Artificial Sequence
  Synthetic
VNNCLLMTRAHCTSY

SEQ ID NO: 135 - protein, 15 aa, Artificial Sequence
  Synthetic
LPCWAFAVNPLHCGD

SEQ ID NO: 136 - protein, 15 aa, Artificial Sequence
  Synthetic
VNNCLLMTRAHCTSY

SEQ ID NO: 137 - protein, 15 aa, Artificial Sequence
  Synthetic
SSCPHPLGLTGCNDK

SEQ ID NO: 138 - protein, 15 aa, Artificial Sequence
  Synthetic
NKCFVHPLAMPNCNS

SEQ ID NO: 139 - protein, 15 aa, Artificial Sequence
  Synthetic
FVGCHSVYVSGCLRA

SEQ ID NO: 140 - protein, 15 aa, Artificial Sequence
  Synthetic
NMCHPPHNIYSICNM

SEQ ID NO: 141 - protein, 14 aa, Artificial Sequence
  Synthetic
LTCHLLPGLTLHTK

SEQ ID NO: 142 - protein, 15 aa, Artificial Sequence
  Synthetic
RTCHPLPGLTLHCTK

SEQ ID NO: 143 - protein, 15 aa, Artificial Sequence
  Synthetic
HPLCFESMKNCFPNY

SEQ ID NO: 144 - protein, 15 aa, Artificial Sequence
  Synthetic
TTCHPLSFTHNYCIT

SEQ ID NO: 145 - protein, 15 aa, Artificial Sequence
  Synthetic
RDCGFDAVRADCLFG

SEQ ID NO: 146 - protein, 15 aa, Artificial Sequence
  Synthetic
RTCSTHPLTMPQCNY

SEQ ID NO: 147 - protein, 15 aa, Artificial Sequence
  Synthetic
MKCHPLQLTGNTCSM

SEQ ID NO: 148 - protein, 15 aa, Artificial Sequence
  Synthetic
SGCPHPLQLITCSTA

SEQ ID NO: 149 - protein, 15 aa, Artificial Sequence
  Synthetic
KCFPAFHDGPLACAS

SEQ ID NO: 150 - protein, 15 aa, Artificial Sequence
  Synthetic
LKCQHPLPMSHCQPQ

SEQ ID NO: 151 - protein, 15 aa, Artificial Sequence
  Synthetic
AFCGFSVIHPLCSGA

SEQ ID NO: 152 - protein, 15 aa, Artificial Sequence
  Synthetic
SVHCAVLKLDGCLGW

SEQ ID NO: 153 - protein, 15 aa, Artificial Sequence
  Synthetic
TLPCHPIMVLGCTPM

SEQ ID NO: 154 - protein, 15 aa, Artificial Sequence
  Synthetic
HYPCMKYNPLNCSMS

SEQ ID NO: 155 - protein, 15 aa, Artificial Sequence
  Synthetic
LKCPHPLSLNGCTLK

SEQ ID NO: 156 - protein, 15 aa, Artificial Sequence
  Synthetic
VYSCMANNPLDCFTQ

SEQ ID NO: 157 - protein, 15 aa, Artificial Sequence
  Synthetic
PICHPLVTLMSYCNK

SEQ ID NO: 158 - protein, 15 aa, Artificial Sequence
  Synthetic
DWCSFWAGQSVWCTS

SEQ ID NO: 159 - protein, 15 aa, Artificial Sequence
  Synthetic
STCHPLTPFHDKCRY

SEQ ID NO: 160 - protein, 15 aa, Artificial Sequence
  Synthetic
PVCPPLVTLMSYCNK

SEQ ID NO: 161 - protein, 15 aa, Artificial Sequence
  Synthetic
STCHPLPTLMPYCNS

SEQ ID NO: 162 - protein, 15 aa, Artificial Sequence
  Synthetic
FPLCGIGPAFCDTTV

SEQ ID NO: 163 - protein, 15 aa, Artificial Sequence
  Synthetic
PTCHPLVLSVPCPKI

SEQ ID NO: 164 - protein, 15 aa, Artificial Sequence
  Synthetic
GPLCDYFVFYSCRGS

SEQ ID NO: 165 - protein, 15 aa, Artificial Sequence
  Synthetic
HTCYHPLKLGQCEMF

SEQ ID NO: 166 - protein, 15 aa, Artificial Sequence
  Synthetic
RTCIHPLPLHQCHKP

SEQ ID NO: 167 - protein, 15 aa, Artificial Sequence
  Synthetic
ACHPINFNSIVYCNN

SEQ ID NO: 168 - protein, 15 aa, Artificial Sequence
  Synthetic
SHPCSVVNLPGCEPD

SEQ ID NO: 169 - protein, 15 aa, Artificial Sequence
  Synthetic
LCHPLVLSAWESCSS

SEQ ID NO: 170 - protein, 15 aa, Artificial Sequence
  Synthetic
LCAPLVLSAWESCSS

SEQ ID NO: 171 - protein, 15 aa, Artificial Sequence
  Synthetic
LCHALVLSAWESCSS

SEQ ID NO: 172 - protein, 15 aa, Artificial Sequence
  Synthetic
LCHPAVLSAWESCSS

SEQ ID NO: 173 - protein, 15 aa, Artificial Sequence
  Synthetic
LCHPLALSAWESCSS

SEQ ID NO: 174 - protein, 15 aa, Artificial Sequence
  Synthetic
LCHPLVASAWESCSS

SEQ ID NO: 175 - protein, 15 aa, Artificial Sequence
  Synthetic
LCHPLVLSAAESCSS

SEQ ID NO: 176 - protein, 15 aa, Artificial Sequence
  Synthetic
LCHPLVLSAWASCSS

SEQ ID NO: 177 - protein, 12 aa, Artificial Sequence
  Synthetic
CHPLVLSAWESC

SEQ ID NO: 178 - protein, 5 aa, Artificial Sequence
  Synthetic
HPLVL

SEQ ID NO: 179 - skipped

SEQ ID NO: 180 - protein, 15 aa, Artificial Sequence
  Synthetic
LEGWCLHPLCLWGAG

SEQ ID NO: 181 - protein, 15 aa, Artificial Sequence
  Synthetic
LEGACLHPLCLWGAG

SEQ ID NO: 182 - protein, 15 aa, Artificial Sequence
  Synthetic
LEGWCAHPLCLWGAG

SEQ ID NO: 183 - protein, 15 aa, Artificial Sequence
  Synthetic
LEGWCLAPLCLWGAG

SEQ ID NO: 184 - protein, 15 aa, Artificial Sequence
  Synthetic
LEGWCLHALCLWGAG

SEQ ID NO: 185 - protein, 15 aa, Artificial Sequence
  Synthetic
LEGWCLHPACLWGAG

SEQ ID NO: 186 - protein, 15 aa, Artificial Sequence
  Synthetic
LEGWCLHPLCAWGAG

SEQ ID NO: 187 - protein, 15 aa, Artificial Sequence
  Synthetic
LEGWCLHPLCLAGAG

SEQ ID NO: 188 - protein, 6 aa, Artificial Sequence
  Synthetic
CLHPLC

SEQ ID NO: 189 - protein, 6 aa, Artificial Sequence
  Synthetic
QGQSGS

SEQ ID NO: 190 - protein, 5 aa, Artificial Sequence
  Synthetic
GQSGS

SEQ ID NO: 191 - protein, 4 aa, Artificial Sequence
  Synthetic
QSGS

SEQ ID NO: 192-193 - skipped (2 entries)

SEQ ID NO: 194 - protein, 7 aa, Artificial Sequence
  Synthetic
QGQSGQG

SEQ ID NO: 195 - protein, 6 aa, Artificial Sequence
  Synthetic
GQSGQG

SEQ ID NO: 196 - protein, 5 aa, Artificial Sequence
  Synthetic
QSGQG

SEQ ID NO: 197 - protein, 4 aa, Artificial Sequence
  Synthetic
SGQG

SEQ ID NO: 198-199 - skipped (2 entries)

SEQ ID NO: 200 - protein, 6 aa, Artificial Sequence
  Synthetic
QGQSGQ

SEQ ID NO: 201 - protein, 5 aa, Artificial Sequence
  Synthetic
GQSGQ

SEQ ID NO: 202 - protein, 4 aa, Artificial Sequence
  Synthetic
QSGQ

SEQ ID NO: 203-204 - skipped (2 entries)

SEQ ID NO: 205 - protein, 121 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTMTNMDPVDTATYYCVQIDYGNDYAFTYWGQGTLVTVSS

SEQ ID NO: 206 - protein, 121 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 207 - protein, 112 aa, Artificial Sequence
  Synthetic
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 208 - protein, 112 aa, Artificial Sequence
  Synthetic
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPYTFGQGTKLEIK

SEQ ID NO: 209 - protein, 112 aa, Artificial Sequence
  Synthetic
DIVMTQSPLSLPVTPGEPASISCRSSQSLLHSNGITYLYWYLQKPGQSPQLLIYQMSNRASGVPDRFSGSGSGTDFTLKISRVEAEDVGVYYCAQNLELPYTFGQGTKLEIK

SEQ ID NO: 210 - protein, 112 aa, Artificial Sequence
  Synthetic
DIVMTQSPLSLPVTPGEPASISCRSSQSLLHSNGITYLYWYLQKPGQSPQLLIYQMSNRASGVPDRFSSSGSGTDFTLKISRVEAEDVGVYYCAQNLELPYTFGQGTKLEIK

SEQ ID NO: 211 - protein, 152 aa, Artificial Sequence
  Synthetic
LCHPAVLSAWESCSSGGGSSGGSISSGLLSGRSDNHGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 212 - protein, 152 aa, Artificial Sequence
  Synthetic
LCHPLVASAWESCSSGGGSSGGSISSGLLSGRSDNHGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 213 - protein, 156 aa, Artificial Sequence
  Synthetic
LCHPAVLSAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 214 - protein, 156 aa, Artificial Sequence
  Synthetic
LCHPLVASAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 215 - protein, 146 aa, Artificial Sequence
  Synthetic
LCHPAVLSAWESCSSGGGSSGGSGGGSGGGSGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 216 - protein, 451 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 259 aa, Artificial Sequence
  Synthetic
LCHPAVLSAWESCSSGGGSSGGSISSGLLSGRSDNHGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 259 aa, Artificial Sequence
  Synthetic
LCHPLVASAWESCSSGGGSSGGSISSGLLSGRSDNHGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 263 aa, Artificial Sequence
  Synthetic
LCHPAVLSAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 263 aa, Artificial Sequence
  Synthetic
LCHPLVASAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 253 aa, Artificial Sequence
  Synthetic
LCHPLVASAWESCSSGGGSSGGSGGGSGGGSGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 451 aa, Artificial Sequence; identical to SEQ ID NO: 216
  Synthetic

SEQ ID NO: 223 - protein, 13 aa, Artificial Sequence
  Synthetic
GGGSSGGSGGSGG

SEQ ID NO: 224 - protein, 442 aa, Artificial Sequence
  Synthetic
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 225 - protein, 264 aa, Artificial Sequence
  Synthetic
QGQSGSGIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 445 aa, Artificial Sequence
  Synthetic
EVQLVESGGGLVQPGGSLRLSCAASGFTFSGYAMSWVRQAPGKGLEWVAYISNSGGNAHYADSVKGRFTISRDNSKNTLYLQMNSLRAEDTAVYYCTREDYGTSPFVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 227 - protein, 265 aa, Artificial Sequence
  Synthetic
QGQSGQGTSYCSIEHYPCNTHHGGGSSGGSISSGLLSGRSDNPGGGSDIQLTQSPSSLSASVGDRVTITCRASESVDAYGISFMNWFQQKPGKAPKLLIYAASNQGSGVPSRFSGSGSGTDFTLTISSMQPEDFATYYCQQSKDVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 218 aa, Artificial Sequence
  Synthetic
DIQLTQSPSSLSASVGDRVTITCRASESVDAYGISFMNWFQQKPGKAPKLLIYAASNQGSGVPSRFSGSGSGTDFTLTISSMQPEDFATYYCQQSKDVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 8 aa, Artificial Sequence
  Synthetic
GGGSSGGS

(19) United States
(12) Patent Application Publication
(10) Pub. No.: US 2021/0020264 A1
(43) Pub. Date: Jan. 21, 2021

(54) SYSTEMS AND METHODS FOR QUANTITATIVE PHARMACOLOGICAL MODELING OF ACTIVATABLE ANTIBODY SPECIES IN MAMMALIAN SUBJECTS

(71) Applicant: CytomX Therapeutics, Inc., South San Francisco, CA (US)

(72) Inventors: Mark Anthony Stroh, South San Francisco, CA (US); Jason Gary Sagert, South San Francisco, CA (US); Joshua F. Apgar, Lincoln, MA (US); Bjorn L. Millard, Lincoln, MA (US); Lin Lin, Lincoln, MA (US); John M. Burke, Lincoln, MA (US); W. Michael Kavanaugh, South San Francisco, CA (US)

(21) Appl. No.: 16/981,281

(22) PCT Filed: Mar. 20, 2019
(86) PCT No.: PCT/US2019/023161

§ 371 Date: Sep. 16, 2020

Related U.S. Application Data
(60) Provisional application No. 62/716,870, filed on Aug. 9, 2018.
(60) Provisional application No. 62/657,549, filed on Apr. 13, 2018.
(60) Provisional application No. 62/645,737, filed on Mar. 20, 2018.

(51) Int. Cl. G16B 5/30

(52) U.S. Cl. CPC G16B 5/30

(57) ABSTRACT

The present invention provides methods and systems useful for modeling the pharmacology of an activated binding polypeptide or activatable antibody in a mammalian subject.

### CROSS-REFERENCE TO RELATED APPLICATIONS

This application claims the benefit of U.S. Provisional Application No. 62/645,737, filed on Mar. 20, 2018, No. 62/657,549, filed on Apr. 13, 2018, and No. 62/716,870, filed on Aug. 9, 2018, the contents of which are incorporated herein by reference in their entireties.

### REFERENCE TO SEQUENCE LISTING

The Sequence Listing submitted electronically concurrently herewith pursuant 37 C.F.R. § 1.821 in computer readable form (ASCII format) via EFS-Web as file name CYTX_049_PCT_ST25.txt is incorporated herein by reference. The ASCII copy of the Sequence Listing was created on Mar. 19, 2019 and is 89 kilobytes in size.

### BACKGROUND

Antibody-based therapies have been demonstrated to be effective in the treatment of several diseases, including many types of cancers. However, in some cases, these therapeutic antibodies have on-target toxicities due to the broad expression of the target in both diseased and healthy tissues. Other limitations such as rapid clearance from the circulation following administration further hinder their effective use as a therapy. Activatable antibodies are designed to selectively activate and bind when exposed to the microenvironment of a target tissue, such as in a tumor, thus potentially reducing toxicities associated with antibody binding to widely expressed binding targets. Methods and systems of modeling the distribution, pharmacodynamics, and pharmacokinetics of activatable antibodies in a subject are desired.

### SUMMARY OF THE INVENTION

One aspect of this disclosure pertains to methods of preparing a quantitative systems pharmacology model for predicting the disposition of an activatable antibody administered to a subject. Such methods may be characterized by the following operations: (a) providing at least one relationship or parameter characterizing mass transfer of activatable antibody and/or activated antibody between a non-target compartment of the subject and a target compartment of the subject; (b) providing a plurality of relationships and/or parameters characterizing reactions in the non-target compartment and/or the target compartment; (c) programming a computational system with (i) a rate constant for the relationship or the parameter characterizing the mass transfer of activatable antibody and/or activated antibody, and (ii) a rate constant for the relationship or the parameter characterizing at least one of the reactions in the non-target compartment and/or the target compartment.

In certain embodiments, the target compartment includes a target to which an antibody or an antigen binding fragment (AB) specifically binds. In certain embodiments, the activatable antibody includes an AB and a prodomain that includes a masking moiety (MM) and a cleavable moiety (CM). The activatable antibody has a reduced binding affinity to the target compared to the AB. In certain embodiments, the activated antibody includes an AB that includes at least one prodomain that no longer masks the AB or lacks at least one prodomain relative to the activatable antibody, where the activated antibody has a higher binding affinity to the target compared to the activatable antibody.

In certain embodiments, the activatable antibody is an activatable bispecific antibody that includes (1) an AB1 and a first prodomain that includes a first masking moiety (MM1) and a first cleavable moiety (CM1) and (2) an AB2 and a second prodomain that includes a second masking moiety (MM2) and a second cleavable moiety (CM2). The AB1 and AB2 can each specifically bind to a different target. In some embodiments, the AB1 can specifically bind to a target antigen expressed on a target cell, such as a tumor cell, and the AB2 can specifically bind to a target antigen expressed on a T-cell. The activatable bispecific antibody has a reduced binding affinity to the respective targets compared to the AB1 and AB2 when both prodomains are masking their respective AB domains. In certain embodiments, the activated bispecific antibody includes an AB1 or AB2 that includes at least one prodomain that no longer masks the respective AB1 or AB2 or lacks at least one prodomain relative to the activatable bispecific antibody, where the activated bispecific antibody has a higher binding affinity to the target of the unmasked AB1 or AB2 compared to the activatable bispecific antibody. In certain embodiments, the activated bispecific antibody includes an AB1 or AB2 where both the AB1 and the AB2 include at least one prodomain that no longer masks the respective AB1 or AB2 or lacks at least one prodomain relative to the activatable bispecific antibody, where the activated bispecific antibody has a higher binding affinity to the targets of the unmasked AB1 or AB2 compared to the activatable bispecific antibody. In such embodiments, the activated bispecific antibody can bind to both antigens simultaneously, thereby forming a trimer of the activated bispecific antibody, the target cell of AB1, and the target cell (e.g. T-cell) of AB2 where the two cells are in physical proximity.

In certain embodiments, at least one of the reactions is a reaction that converts the activatable antibody to the activated antibody which has increased affinity to binding the target compared to the activatable antibody by (i) a change of conformation of at least one prodomain of the activatable antibody with respect to the AB in the activatable antibody or (ii) a cleavage of at least one prodomain away from the AB. The conversion results in increased affinity to binding the target by the AB compared to the activatable antibody.

In some cases, the resulting computational system is programmed to (i) solve a system of expressions under a defined set of pharmacological conditions, where the system of expressions includes the at least one relationship or parameter characterizing mass transfer of activatable antibody and/or activated antibody, and the plurality of relationships and/or parameters characterizing the reactions, and (ii) output of one or more predicted pharmacodynamics and/or pharmacokinetic parameter values in the subject after administration of the activatable antibody to the subject under the defined set of pharmacological conditions.

In certain embodiments, the methods may include one or both of the following operations: determining a rate constant for the relationship or the parameter characterizing the mass transfer of activatable antibody and/or activated antibody by using first measurements of the activatable antibody and/or the activated antibody in one or more test subjects or in vitro; and determining a rate constant for the relationship or the parameter characterizing at least one of the reactions by using second measurements of the activatable antibody and/or the activated antibody in the one or more test subjects or in vitro. The first measurements of the activatable antibody and/or activated antibody may include measurements of time-varying values of concentrations of the activatable antibody and/or the activated antibody in samples taken from the one or more test subjects who were administered one or more doses of the activatable antibody. The second measurements of the activatable antibody and/or the activated antibody may be measurements of time-varying values of concentrations of the activatable antibody and/or activated antibody in samples taken from the one or more test subjects who were administered one or more doses of the activatable antibody.

In certain embodiments, the target compartment represents a tumor in the subject, wherein the tumor expresses the target. In certain embodiments, the non-target compartment represents a portion of the subject that initially receives, upon administration, the activatable antibody. For example, the non-target compartment may represent, at least, a plasma compartment of the subject.

In certain embodiments, a first reaction of the reactions takes place in the target compartment and a second reaction of the reactions takes place in the non-target compartment. In certain embodiments, the relationship characterizing mass transfer of the activatable antibody and/or the activated antibody is a rate expression employing concentrations of the activatable antibody and/or the activated antibody in the non-target compartment. In certain embodiments, the methods additionally include providing a relationship or parameter characterizing mass transfer of the activatable antibody and/or the activated antibody between the non-target compartment of the subject and a second non-target compartment of the subject. The second non-target compartment may represent one or more non-tumor organs or tissues in the subject.

In certain embodiments, at least one of the plurality of relationships characterizing the reactions in the non-target compartment and/or the target compartment includes cleavage of the CM of the activated antibody or the activatable antibody. In certain embodiments, at least one of the plurality of relationships characterizing the reactions in the non-target compartment and/or the target compartment comprises binding of the activated antibody to the target. In certain embodiments, at least one of the plurality of relationships characterizing the reactions in the non-target compartment and/or the target compartment comprises unmasking of the AB of the uncleaved activatable antibody resulting in reduced inhibition to binding the target by the AB. In certain embodiments, at least one of the plurality of relationships characterizing the reactions in the non-target compartment and/or the target compartment comprises a relationship for a rate of cleaving the CM as a function of concentration of a protease, where the CM is a substrate for the protease. In certain embodiments, the plurality of relationships and/or parameters characterizing the reactions in the non-target compartment and/or the target compartment includes a relationship for a rate of target expression or an amount of the target.

In certain embodiments, the pharmacological conditions include one or more of: a dose of the activatable antibody, a frequency of dose of the activatable antibody, other medicaments administered concurrently with the activatable antibody, an activatable antibody binding affinity, an activated antibody binding affinity, a masking efficiency of the MM, a rate of cleavage of the CM, a target concentration in the target compartment, and a partition coefficient of the activatable antibody between two or more compartments. In certain embodiments, the pharmacodynamics and pharmacokinetic parameter values include one or more of: a target occupancy by the activatable antibody in a target compartment; a target occupancy by the activatable antibody in a peripheral compartment; a therapeutic window; a target mediated drug disposition in a target compartment; a target mediated drug disposition in a peripheral compartment; a target mediated drug disposition in a plasma compartment; a concentration of activated antibody and/or activatable antibody in a target compartment; a concentration of activated antibody and/or activatable antibody in a plasma compartment; and a concentration of activated antibody and/or activatable antibody in a peripheral compartment.

In certain embodiments, determining the rate constant for the relationship or the parameter characterizing the mass transfer of the activatable antibody and/or the activated antibody includes applying an objective function to evaluate at least time-varying values of concentration of the activatable antibody and/or the activated antibody in samples taken from the one or more test subjects. In certain embodiments, the objective function is a log likelihood function. In certain embodiments, determining the rate constant for the relationship or the parameter characterizing at least one of the reactions includes applying an objective function to evaluate at least time-varying values of concentration of the activatable antibody and/or the activated antibody in samples taken from the one or more test subjects. In certain embodiments, the objective function is a log likelihood function.

In certain embodiments, the system of expressions includes expressions for one or more zero order, first order, and/or second order rate relationships. In certain embodiments, the system of expressions includes: time-dependent differential equations for the activated antibody in the non-target compartment and/or time-dependent differential equations for the activatable antibody in the non-target compartment; and time-dependent differential equations for activated antibody in the target compartment and/or time-dependent differential equations for activatable antibody in the target compartment. In certain embodiments, the system of expressions is configured to, during execution of the quantitative systems pharmacology model, numerically solve time-dependent differential equations to provide: a prediction of a time-dependent concentration or amount of the activated antibody in the non-target compartment and/or a time-dependent concentration or amount of the activatable antibody in the non-target compartment, and a prediction of a time-dependent concentration or amount of the activated antibody in the target compartment and/or a time-dependent concentration or amount of the activatable antibody in the target compartment.

Another aspect of the disclosure pertains to computer program products including a non-transitory computer readable medium on which is provided instructions for causing a computational system to execute a quantitative systems pharmacology model for predicting pharmacodynamics and/or pharmacokinetic parameter values in a subject administered an activatable antibody. The instructions may include instructions for: solving a system of expressions under a defined set of pharmacological conditions; and outputting one or more predicted pharmacodynamics and/or pharmacokinetic parameter values in the subject after administration of the activatable antibody to the subject under the defined set of pharmacological conditions.

In certain embodiments, the system of expressions represents: (a) at least one relationship or parameter characterizing mass transfer of activatable antibody and/or activated antibody between a non-target compartment of the subject and a target compartment of the subject, and (b) a plurality of relationships and/or parameters characterizing reactions in the non-target compartment and/or the target compartment.

In certain embodiments, the target compartment includes a target to which an AB binds. In certain embodiments, the activatable antibody includes an AB and a prodomain that includes a MM and a CM. The activatable antibody has a reduced binding affinity to the target compared to the AB. In certain embodiments, the activated antibody includes an AB that includes at least one prodomain that no longer masks the AB or lacks at least one prodomain relative to the activatable antibody, where the activated antibody has a higher binding affinity to the target compared to the activatable antibody.

In certain embodiments, at least one of the reactions is a reaction that converts the activatable antibody to the activated antibody which has increased affinity to binding the target compared to the activatable antibody. In certain embodiments, the converting step involves a change of conformation of at least one prodomain of the activatable antibody with respect to the AB in the activatable antibody or a cleavage of at least one prodomain away from the AB, whereby the conversion results in increased affinity to binding the target by the AB compared to the activatable antibody.

In certain embodiments, a rate constant for the relationship or the parameter characterizing the mass transfer of activatable antibody and/or activated antibody was determined by using measurements of the activatable antibody and/or the activated antibody in one or more test subjects or in vitro. In certain embodiments, the rate constant for the relationship or the parameter characterizing the mass transfer of the activatable antibody and/or the activated antibody was determined by applying an objective function to evaluate at least time-varying values of concentration of the activatable antibody and/or the activated antibody in samples taken from the one or more test subjects. As an example, the objective function may be a log likelihood function.

In certain embodiments, a rate constant for the relationship or the parameter characterizing at least one of the reactions of activatable antibody and/or activated antibody was determined by using measurements of the activatable antibody and/or the activated antibody in one or more test subjects or in vitro. In certain embodiments, the rate constant for the relationship or the parameter characterizing at least one of the reactions was determined by applying an objective function to evaluate at least time-varying values of concentration of the activatable antibody and/or the activated antibody in samples taken from the one or more test subjects. As an example, the objective function may be a log likelihood function.

In certain embodiments, the target compartment represents a tumor in the subject, wherein the tumor expresses the target. In certain embodiments, the non-target compartment represents a portion of the subject that initially receives, upon administration, the activatable antibody. In some cases, the non-target compartment represents, at least, a plasma compartment of the subject.

In certain embodiments, a first reaction of the reactions takes place in the target compartment and a second reaction of the reactions takes place in the non-target compartment. In certain embodiments, the relationship characterizing mass transfer of the activatable antibody and/or the activated antibody is a rate expression employing concentrations of the activatable antibody and/or the activated antibody in the non-target compartment. In certain embodiments, the system of expressions further represents a relationship or parameter characterizing mass transfer of the activatable antibody and/or the activated antibody between the non-target compartment of the subject and a second non-target compartment of the subject. As an example, the second non-target compartment may represent one or more non-tumor organs or tissues in the subject.

In certain embodiments, at least one of the plurality of relationships characterizing the reactions in the non-target compartment and/or the target compartment includes cleavage of the CM of the activated antibody or the activatable antibody. In certain embodiments, at least one of the plurality of relationships characterizing the reactions in the non-target compartment and/or the target compartment comprises binding of the activated antibody to the target. In certain embodiments, at least one of the plurality of relationships characterizing the reactions in the non-target compartment and/or the target compartment comprises unmasking of the AB of the uncleaved activatable antibody resulting in reduced inhibition to binding the target by the AB. In certain embodiments, at least one of the plurality of relationships characterizing the reactions in the non-target compartment and/or the target compartment comprises a relationship for a rate of cleaving the CM as a function of concentration of a protease, where the CM is a substrate for the protease. In certain embodiments, the plurality of relationships and/or parameters characterizing the reactions in the non-target compartment and/or the target compartment includes a relationship for a rate of target expression or an amount of the target.

In certain embodiments, the pharmacological conditions include one or more of: a dose of the activatable antibody, a frequency of dose of the activatable antibody, other medicaments administered concurrently with the activatable antibody, an activatable antibody binding affinity, an activated antibody binding affinity, a masking efficiency of the MM, a rate of cleavage of the CM, a target concentration in the target compartment, and a partition coefficient of the activatable antibody between two or more compartments. In certain embodiments, the pharmacodynamics and pharmacokinetic parameter values include one or more of: a target occupancy by the activatable antibody in a target compartment; a target occupancy by the activatable antibody in a peripheral compartment; a therapeutic window; a target mediated drug disposition in a target compartment; a target mediated drug disposition in a peripheral compartment; a target mediated drug disposition in a plasma compartment; a concentration of activated antibody and/or activatable antibody in a target compartment; a concentration of activated antibody and/or activatable antibody in a plasma compartment; and a concentration of activated antibody and/or activatable antibody in a peripheral compartment.

In certain embodiments, the rate constant for the relationship or the parameter characterizing the mass transfer of the activatable antibody and/or the activated antibody was determined by applying an objective function (e.g., a log likelihood function) to evaluate at least time-varying values of concentration of the activatable antibody and/or the activated in samples taken from the one or more test subjects. In certain embodiments, the rate constant for the relationship or the parameter characterizing at least one of the reactions was determined by applying an objective function (e.g., a log likelihood function) to evaluate at least time-varying values of concentration of the activatable antibody and/or the activated antibody in samples taken from the one or more test subjects.

In certain embodiments, the system of expressions includes expressions for one or more zero order, first order, and/or second order rate relationships. In certain embodiments, the system of expressions includes: time-dependent differential equations for the activated antibody in the non-target compartment and/or time-dependent differential equations for the activatable antibody in the non-target compartment; and time-dependent differential equations for activated antibody in the target compartment and/or time-dependent differential equations for activatable antibody in the target compartment. In certain embodiments, the system of expressions is configured to, during execution of the quantitative systems pharmacology model, numerically solve time-dependent differential equations to provide: a prediction of a time-dependent concentration or amount of the activated antibody in the non-target compartment and/or a time-dependent concentration or amount of the activatable antibody in the non-target compartment, and a prediction of a time-dependent concentration or amount of the activated antibody in the target compartment and/or a time-dependent concentration or amount of the activatable antibody in the target compartment.

Yet another aspect of the disclosure pertains to methods of predicting pharmacodynamics and/or pharmacokinetic parameter values in a subject after administration of an activatable antibody. Such methods may be characterized by the following operations: inputting a defined set of pharmacological conditions to a quantitative systems pharmacology model having instructions for solving a system of expressions under a defined set of pharmacological conditions; and receiving from the quantitative systems pharmacology model one or more predicted pharmacodynamics and/or pharmacokinetic parameter values in the subject after administration of the activatable antibody to the subject under the defined set of pharmacological conditions.

In certain embodiments, the system of expressions represents: (a) at least one relationship or parameter characterizing mass transfer of activatable antibody and/or activated antibody between a non-target compartment of the subject and a target compartment of the subject, and (b) a plurality of relationships and/or parameters characterizing reactions in the non-target compartment and/or the target compartment.

In certain embodiments, the target compartment includes a target to which an AB binds. In certain embodiments, the activatable antibody includes an AB and a prodomain that includes a MM and a CM. The activatable antibody has a reduced binding affinity to the target compared to the AB. In certain embodiments, the activated antibody includes an AB that includes at least one prodomain that no longer masks the AB or lacks at least one prodomain relative to the activatable antibody, where the activated antibody has a higher binding affinity to the target compared to the activatable antibody.

In certain embodiments, at least one of the reactions is a reaction that converts the activatable antibody to the activated antibody which has increased affinity to binding the target compared to the activatable antibody. In certain embodiments, the converting step involves a change of conformation of at least one prodomain of the activatable antibody with respect to the AB in the activatable antibody or a cleavage of at least one prodomain away from the AB, whereby the conversion results in increased affinity to binding the target by the AB compared to the activatable antibody.

In certain embodiments, the methods additionally include using the one or more predicted pharmacodynamics and pharmacokinetic parameter values to identify or select a therapeutic activatable antibody having a selected susceptibility to cleaving the MM from the AB. In certain embodiments, the methods further include using the one or more predicted pharmacodynamics and pharmacokinetic parameter values to identify or select a treatment regimen for using the activatable antibody to treat a patient.

In certain embodiments, the target compartment represents a tumor in the subject, wherein the tumor expresses the target. In certain embodiments, the non-target compartment represents a portion of the subject that initially receives, upon administration, the activatable antibody. For example, the non-target compartment may represent, at least, a plasma compartment of the subject.

In certain embodiments, a first reaction of the reactions takes place in the target compartment and a second reaction of the reactions takes place in the non-target compartment. In certain embodiments, the relationship characterizing mass transfer of the activatable antibody and/or the activated antibody is a rate expression employing concentrations of the activatable antibody and/or the activated antibody in the non-target compartment. In certain embodiments, the methods additionally include providing a relationship or parameter characterizing mass transfer of the activatable antibody and/or the activated antibody between the non-target compartment of the subject and a second non-target compartment of the subject. The second non-target compartment may represent one or more non-tumor organs or tissues in the subject.

In certain embodiments, at least one of the plurality of relationships characterizing the reactions in the non-target compartment and/or the target compartment includes cleavage of the CM of the activated antibody or the activatable antibody. In certain embodiments, at least one of the plurality of relationships characterizing the reactions in the non-target compartment and/or the target compartment comprises binding of the activated antibody to the target. In certain embodiments, at least one of the plurality of relationships characterizing the reactions in the non-target compartment and/or the target compartment comprises unmasking of the AB of the uncleaved activatable antibody resulting in reduced inhibition to binding the target by the AB. In certain embodiments, at least one of the plurality of relationships characterizing the reactions in the non-target compartment and/or the target compartment comprises a relationship for a rate of cleaving the CM as a function of concentration of a protease, where the CM is a substrate for the protease. In certain embodiments, the plurality of relationships and/or parameters characterizing the reactions in the non-target compartment and/or the target compartment includes a relationship for a rate of target expression or an amount of the target.

In certain embodiments, the system of expressions includes expressions for one or more zero order, first order, and/or second order rate relationships. In certain embodiments, the system of expressions includes: time-dependent differential equations for the activated antibody in the non-target compartment and/or time-dependent differential equations for the activatable antibody in the non-target compartment; and time-dependent differential equations for activated antibody in the target compartment and/or time-dependent differential equations for activatable antibody in the target compartment. In certain embodiments, the system of expressions is configured to, during execution of the quantitative systems pharmacology model, numerically solve time-dependent differential equations to provide: a prediction of a time-dependent concentration or amount of the activated antibody in the non-target compartment and/or a time-dependent concentration or amount of the activatable antibody in the non-target compartment, and a prediction of a time-dependent concentration or amount of the activated antibody in the target compartment and/or a time-dependent concentration or amount of the activatable antibody in the target compartment.

The features described for each of the aspects presented above may be employed in any combination, so long as any two features in a putative combination are not inconsistent with one another. Thus, embodiments of this disclosure include various combinations of the above recited aspects.

### BRIEF DESCRIPTION OF THE FIGURES

FIG. 1A is a schematic depiction of various species of activatable and activated antibodies and conversion pathways therebetween that are modeled in the QSP model of the present disclosure.

FIG. 1B is a schematic depiction of various physiological compartments and the mass transfer pathways therebetween that are modeled in the QSP model of the present disclosure.

FIG. 2A is a flowchart for an exemplary method of generating a QSP model of the present disclosure.

FIG. 2B is a flowchart for an exemplary method of using a QSP model of the present disclosure.

FIGS. 3A-3C are graphs showing exemplary PK studies of anti-CD166 activatable antibodies in cynomolgus monkeys overlaid with an exemplary QSP model of the present disclosure of the PK of the activatable antibody in monkeys.

FIGS. 4A-4E are graphs showing exemplary QSP models of the present disclosure of the PK of anti-CD166 activatable antibodies in humans.

FIG. 5 is a graph showing an exemplary QSP model of the present disclosure of circulating levels of anti-CD166 activatable antibodies in a subject following multiple administrations.

FIG. 6 are graphs showing exemplary QSP models of the flux of anti-CD166 activated antibodies from various physiological compartments.

FIG. 7 is a schematic of an exemplary computer system used to implement the QSP models of the present disclosure.

FIGS. 8A, 8B, and 8C are exemplary pharmacokinetic data of plasma levels of intact anti-PD-L1 activatable antibodies of the present disclosure.

FIGS. 9A, 9B, and 9C are exemplary comparisons of QSP models of the present disclosure of plasma levels of intact anti-PD-L1 activatable antibody following the indicated dosing regimens.

FIGS. 10A and 10B are exemplary graphs of the QSP model of the present disclosure showing the relationships between administered dose of activatable antibody (FIG. 10A) or Cₘᵢₙ of intact activatable antibody (FIG. 10B) and the periphery/tumor cleavage ratio to achieve a specific receptor occupancy in the tumor.

FIGS. 11A through 11D are exemplary modeled pharmacokinetic graphs of the clearance of isotopically-labeled monoclonal and activatable antibodies from plasma.

FIGS. 12A, 12B, and 12C are exemplary comparisons of QSP models of the activated activatable antibody in tumor compartments as compared to the observed amounts of the activatable antibody.

FIGS. 13A and 13B are exemplary pharmacokinetic data and QSP models of the present disclosure of the anti-PD-1 monoclonal antibody and the anti-PD-1 activated activatable antibody in cynomolgus monkeys.

FIG. 14A are exemplary pharmacokinetic data and QSP models of the anti-PD-1 monoclonal antibody pembrolizumab in humans. FIG. 14B is an exemplary QSP model of the present disclosure of the receptor occupancy of the anti-PD-1 monoclonal antibody as a function of the dosage.

FIG. 15 is a schematic depiction of various species of activatable and activated T-cell bispecific antibodies and conversion pathways therebetween that are modeled in the QSP model of the present disclosure.

FIG. 16 is a schematic depiction of various physiological compartments and the mass transfer pathways therebetween that are modeled in the QSP model of the present disclosure as relating to activatable and activated T-cell bispecific antibodies.

FIG. 17 is a schematic depiction of a human QSP model of the present disclosure of circulating levels of anti-PD-1 activatable antibody following its administration at the indicated dosages.

FIG. 18 shows exemplary receptor occupancy (RO) of administered anti-PD-1 activatable antibody in a human tumor based on QSP modeling of the present disclosure and calculated based on patient biopsies.

### DETAILED DESCRIPTION

As used herein, the term “activatable binding polypeptide” refers to a compound that comprises a binding moiety (BM), linked either directly or indirectly, to a prodomain. The term “binding moiety” and “BM” are used interchangeably herein to refer to a polypeptide that is capable of specifically binding to a biological target. When in a form not modified by the presence of the prodomain, the BM is a polypeptide that specifically binds the biological target. The terms “biological target,” “binding target,” and “target” (when used in the context of binding) refer interchangeably herein to polypeptide that may be present in a mammalian subject. The terms “distribution” and “biodistribution” are used interchangeably herein to refer to the location of activated binding polypeptide in a mammalian subject.

As used herein, the term “activatable antibody” refers to an activatable binding polypeptide in which the binding moiety (BM) is an antibody or the antigen-binding fragment thereof (AB). When in a form not modified by the presence of the prodomain, the BM is an antibody or the antigen-binding fragment thereof (AB) that specifically binds the biological target. Examples, definitions, and descriptions provided herein that refer to a binding moiety (BM) are understood to be applicable to embodiments in which the BM is an antibody or antigen-binding fragment thereof (AB). Similarly, examples, definitions, and descriptions provided herein that refer to an antibody or antigen-binding fragment thereof (AB) are understood to be applicable to other BM embodiments where appropriate.

As used herein, the term “prodomain” refers to a peptide, which comprises a masking moiety (MM) and a cleavable moiety (CM). The prodomain functions to mask the BM or AB until the activatable binding polypeptide is exposed to an activation condition. As used herein, the terms “masking moiety” and “MM”, are used interchangeably herein to refer to a peptide that, when positioned proximal to the BM or AB, interferes with binding of the BM or AB to the biological target. The terms “cleavable moiety” and “CM” are used interchangeably herein to refer to a peptide that is susceptible to cleavage (e.g., an enzymatic substrate, and the like), bond reduction (e.g., reduction of disulfide bond(s), and the like), or other change in physical conformation. The CM is positioned relative to the MM and BM or AB, such that cleavage, or other change in its physical conformation, causes release of the MM from its position proximal to the BM or AB. The term “activation condition” refers to the condition that triggers unmasking of the BM or AB, and results in generation of an “activated binding polypeptide” (or “activated BP”), or an “activated antibody.” Unmasking of the BM or AB typically results in an activated binding polypeptide or activated antibody having greater binding affinity for the biological target as compared to the corresponding activatable binding polypeptide or activatable antibody, respectively. Typically, the activatable binding polypeptide or activatable antibody specifically binds, in vivo or in vitro, a biological target. The terms “peptide,” “polypeptide,” and “protein” are used interchangeably herein to refer to a polymer comprising naturally occurring or non-naturally occurring amino acid residues or amino acid analogues.

Activatable binding polypeptides or activatable antibodies that are suitable for use in the practice of the present invention may comprise the BM or AB and prodomain components, CM and MM, in a variety of linear or cyclic configurations (via, for example, a cysteine-cysteine disulfide bond), and may further comprise one or more optional linker moieties through which any two or more of the BM or AB, CM, and/or MM moieties may be bound indirectly to each other. Linkers suitable for use in the activatable binding polypeptides employed in the practice of the invention may be any of a variety of lengths. Suitable linkers include those having a length in the range of from about 1 to about 20 amino acids, or from about 1 to about 19 amino acids, or from about 1 to about 18 amino acids, or from about 1 to about 17 amino acids, or from about 1 to about 16 amino acids, or from about 1 to about 15 amino acids, or from about 2 to about 15 amino acids, or from about 3 to about 15 amino acids, or from about 3 to about 14 amino acids, or from about 3 to about 13 amino acids, or from about 3 to about 12 amino acids. In some embodiments, the ABP comprises one or more linkers comprising 1, 2, 3, 4, 5, 6, 7, 8, 9, 10, 11, 12, 13, 14, 15, 16, 17, 18, 19, or 20 amino acids. Typically, the linker is a flexible linker.

Exemplary flexible linkers include glycine homopolymers (G)ₙ, wherein n is an integer that is at least 1, glycine-serine polymers, including, for example, (GS)ₙ (wherein n is an integer that is at least 1), (GSGGS)ₙ (SEQ ID NO:68) (wherein n is an integer that is at least 1), (GGGS)ₙ (SEQ ID NO:69) (wherein n is an integer that is at least 1), GGSG (SEQ ID NO:70), GGSGG (SEQ ID NO:71), GSGSG (SEQ ID NO:72), GSGGG (SEQ ID NO:73), GGGSG (SEQ ID NO:74), GSSSG (SEQ ID NO:75), GSSGGSGGSGGSG (SEQ ID NO:76), GSSGGSGGSGG (SEQ ID NO:77), GSSGGSGGSGGS (SEQ ID NO:78), GSSGGSGGSGGSGGGS (SEQ ID NO:79), GSSGGSGGSG (SEQ ID NO:80), GSSGGSGGSGS (SEQ ID NO:81), GGGS (SEQ ID NO:82), GSSGT (SEQ ID NO:83), GSSG (SEQ ID NO:84), GGGSSGGSGGSGG (SEQ ID NO:223), GGS, and the like, and additionally, a glycine-alanine polymer, an alanine-serine polymer, and other flexible linkers known in the art.

Illustrative activatable binding polypeptide configurations include, for example, in either N- to C-terminal direction or C- to N-terminal direction:
(MM)-(CM)-(BM)
(BM)-(CM)-(MM)
(MM)-L₁-(CM)-(BM)
(MM)-L₁-(CM)-L₂-(BM)
cyclo[L₁-(MM)-L₂-(CM)-L₃-(BM)]
(MM)-(CM)-(AB)
(AB)-(CM)-(MM)
(MM)-L₁-(CM)-(AB)
(MM)-L₁-(CM)-L₂-(AB)
cyclo[L₁-(MM)-L₂-(CM)-L₃-(AB)]

wherein each of L₁, L₂, and L₃ is a linker peptide that may be identical or different.

An activatable binding polypeptide or activatable antibody can also include a spacer located, for example, at the amino terminus of the prodomain. In some embodiments, the spacer is joined directly to the MM of the activatable binding polypeptide or activatable antibody. In some embodiments, the spacer is joined directly to the MM of the activatable binding polypeptide or activatable antibody in the structural arrangement from N-terminus to C-terminus of spacer-MM-CM-BM or spacer-MM-CM-AB, respectively. An example of a spacer joined directly to the N-terminus of MM of the activatable antibody is selected from the group consisting of QGQSGS (SEQ ID NO: 189); GQSGS (SEQ ID NO: 190); QSGS (SEQ ID NO: 191); SGS; GS; S; QGQSGQG (SEQ ID NO: 194); GQSGQG (SEQ ID NO: 195); QSGQG (SEQ ID NO: 196); SGQG (SEQ ID NO: 197); GQG; QG; G; QGQSGQ (SEQ ID NO: 200); GQSGQ (SEQ ID NO: 201); QSGQ (SEQ ID NO: 202); SGQ; GQ; and Q.

In some embodiments, the spacer includes at least the amino acid sequence QGQSGS (SEQ ID NO: 189). In some embodiments, the spacer includes at least the amino acid sequence GQSGS (SEQ ID NO: 190). In some embodiments, the spacer includes at least the amino acid sequence QSGS (SEQ ID NO: 191). In some embodiments, the spacer includes at least the amino acid sequence SGS. In some embodiments, the spacer includes at least the amino acid sequence GS. In some embodiments, the spacer includes at least the amino acid sequence S. In some embodiments, the spacer includes at least the amino acid sequence QGQSGQG (SEQ ID NO: 194). In some embodiments, the spacer includes at least the amino acid sequence GQSGQG (SEQ ID NO: 195). In some embodiments, the spacer includes at least the amino acid sequence QSGQG (SEQ ID NO: 196). In some embodiments, the spacer includes at least the amino acid sequence SGQG (SEQ ID NO: 197). In some embodiments, the spacer includes at least the amino acid sequence GQG. In some embodiments, the spacer includes at least the amino acid sequence QG. In some embodiments, the spacer includes at least the amino acid sequence G. In some embodiments, the spacer includes at least the amino acid sequence QGQSGQ (SEQ ID NO: 200). In some embodiments, the spacer includes at least the amino acid sequence GQSGQ (SEQ ID NO: 201). In some embodiments, the spacer includes at least the amino acid sequence QSGQ (SEQ ID NO: 202). In some embodiments, the spacer includes at least the amino acid sequence SGQ. In some embodiments, the spacer includes at least the amino acid sequence GQ. In some embodiments, the spacer includes at least the amino acid sequence Q. In some embodiments, the activatable antibody does not include a spacer sequence.

Activatable binding polypeptides that are suitable for use in the binding polypeptide employed herein include any of the activatable binding polypeptides, modified antibodies, and activatable antibodies described in WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, WO 2018/085555, WO 2018/165619, PCT/US2018/048965, PCT/US2018/055733, PCT/US2018/055717, PCT/US2018/067740, and PCT/US2019/021449 each of which is incorporated herein by reference in its entirety.

Activatable antibodies that are suitable for use in the binding polypeptide employed herein include any of the activatable antibodies described in WO 2009/025846, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, WO 2018/085555, WO 2018/165619, PCT/US2018/048965, PCT/US2018/055733, PCT/US2018/055717, PCT/US2018/067740, and PCT/US2019/021449 each of which is incorporated herein by reference in its entirety.

Typically, the prodomain is linked, either directly or indirectly, to the BM or AB via the CM of the prodomain. The CM may be designed to be cleaved by upregulated proteolytic activity (i.e., the activation condition) in tissue, such as those present in many cancers. Thus, activatable binding polypeptides or activatable antibodies may be designed so they are predominantly activated at a target treatment site where proteolytic activity and the desired biological target are co-localized.

Cleavable moieties suitable for use in activatable binding polypeptides of the present invention include those that are a substrate for a protease. Usually, the protease is an extracellular protease. Suitable substrates may be readily identified using any of a variety of known techniques, including those described in U.S. Pat. Nos. 7,666,817, 8,563,269, PCT Publication No. WO 2014/026136, Boulware, et al., “Evolutionary optimization of peptide substrates for proteases that exhibit rapid hydrolysis kinetics,” Biotechnolo. Bioeng. (2010) 106.3: 339-46, each of which is hereby incorporated by reference in its entirety. Exemplary substrates that are suitable for use as a cleavable moiety include, for example, those that are substrates cleavable by any one or more of the following proteases: an ADAM, an ADAM-like, or ADAMT5 (such as, for example, ADAM8, ADAMS, ADAM10, ADAM12, ADAM15, ADAM17/TACE, ADAMDEC1, ADAMTS1, ADAMTS4, ADAMTS5); an aspartate protease (such as, for example, BACE, Renin, and the like); an aspartic cathepsin (such as, for example, Cathepsin D, Cathepsin E, and the like); a caspase (such as, for example, Caspase 1, Caspase 2, Caspase 3, Caspase 4, Casepase 5, Caspase 6, Caspase 7, Caspase 7, Caspase 8, Caspase 9, Caspase 10, Caspase 14, and the like); a cysteine proteinase (such as, for example, Cruzipain, Legumain, Otubain-2, and the like); a kallikrein-related peptidase (KLK) (such as, for example, KLK4, KLK5, KLK6, KLK7, KLK8, KLK10, KLK11, KLK13, KLK14, and the like); a metallo proteinase (such as, for example, Meprin, Neprilysin, prostate-specific membrane antigen (PSMA), bone morphogenetic protein 1 (BMP-1), and the like); a matrix metalloproteinase (MMP) (such as, for example, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, and the like); a serine protease (such as, for example, activated protein C, Cathepsin A, Cathepsin G, Chymase, a coagulation factor protease (such as, for example, FVIIa, FIXa, FXa, FXIa, FXIIa, and the like)); elastase, Granzyme B, Guanidinobenzoatase, HtrA1, Human Neutrophil Elastase, Lactoferrin, Marapsin, NS3/4A, PACE4, Plasmin, prostate-specific antigen (PSA), tissue plasminogen activator (tPA), Thrombin, Tryptase, urokinase (uPA), a Type II transmembrane Serine Protease (TTSP) (such as, for example, DESC1, DPP-4, FAP, Hepsin, Matriptase-2, MT-SP1/Matriptase, TMPRSS2, TMPRSS3, TMPRSS4, and the like), and the like. Exemplary CMs that are suitable for use in the activatable binding polypeptides of the present invention include those described in, for example, WO 2010/081173, WO 2015/048329, WO 2015/116933, and WO 2016/118629, each of which is incorporated herein by reference in its entirety. Illustrative CMs are provided herein as SEQ ID Nos: 1-67.

The MM is selected such that it reduces the ability of the BM to specifically bind the biological target. As such, the dissociation constant (K_{d}) of the activatable binding polypeptide toward the biological target is usually greater than the K_{d} of the corresponding activated binding polypeptide to the biological target. The MM can inhibit the binding of the activatable binding polypeptide to the biological target in a variety of ways. For example, the MM can bind to the BM thereby inhibiting binding of the activatable binding polypeptide to the biological target. The MM can allosterically or sterically inhibit binding of the activatable binding polypeptide to biological target. In some embodiments, the MM binds specifically to the BM. Suitable MMs may be identified using any of a variety of known techniques. For example, peptide MMs may be identified using the methods described in U.S. Patent Application Publication Nos. 2009/0062142 and 2012/0244154, and PCT Publication No. WO 2014/026136, each of which is hereby incorporated by reference in their entirety.

In some embodiments, the MM is selected such that binding of the activatable binding polypeptide to the biological target is reduced, relative to binding of the corresponding BM (i.e., without the prodomain) to the same target, by at least about 50%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, and even 100%, for at least about 2 hours, or at least about 4 hours, or at least about 6 hours, or at least about 8 hours, or at least about 12 hours, or at least about 24 hours, or at least about 28 hours, or at least about 30 hours, or at least about 36 hours, or at least about 48 hours, or at least about 60 hours, or at least about 72 hours, or at least about 84 hours, or at least about 96 hours, or at least about 5 days, or at least about 10 days, or at least about 15 days, or at least about 30 days, or at least about 45 days, or at least about 60 days, or at least about 90 days, or at least about 120 days, or at least about 150 days, or at least about 180 days, or at least about 1 month, or at least about 2 months, or at least about 3 months, or at least about 4 months, or at least about 5 months, or at least about 6 months, or at least about 7 months, or at least about 8 months, or at least about 9 months, or at least about 10 months, or at least about 11 months, or at least about 12 months or more.

Typically, the MM is selected such that the K_{d} of the activatable binding polypeptide towards the biological target is at least about 2, about 3, about 4, about 5, about 10, about 25, about 50, about 100, about 250, about 500, about 1,000, about 2,500, about 5,000, about 10,000, about 100,000, about 500,000, about 1,000,000, about 5,000,000, about 10,000,000, about 50,000,000, or greater, or in the range of from about 5 to about 10, or from about 10 to about 100, or from about 10 to about 1,000, or from about 10 to about 10,000 or from about 10 to about 100,000, or from about 10 to about 1,000,000, or from about 10 to about 10 to about 10,000,000, or from about 100 to about 1,000, or from about 100 to about 10,000, or from about 100 to about 100,000, or from about 100 to about 1,000,000, or from about 100 to about 10,000,000, or from about 1,000 to about 10,000, or from about 1,000 to about 100,000, or from about 1,000 to about 1,000,000, or from about 1,000 to about 10,000,000, or from about 10,000 to about 100,000, or from about 10,000 to about 1,000,000, or from about 10,000 to about 10,000,000 or from about 100,000 to about 1,000,00, or 100,000 to about 10,000,000 times greater than the K_{d} of the BM (i.e., not modified with a prodomain).

Conversely, the MM is selected such that the K_{d} of the BM (i.e., not modified with a prodomain) towards the biological target is at least about 2, about 3, about 4, about 5, about 10, about 25, about 50, about 100, about 250, about 500, about 1,000, about 2,500, about 5,000, about 10,000, about 100,000, about 500,000, about 1,000,000, about 5,000,000, about 10,000,000, about 50,000,000, or more times lower than the binding affinity of the corresponding activatable binding polypeptide; or in the range of from about 5 to about 10, or from about 10 to about 100, or from about 10 to about 1,000, or from about 10 to about 10,000 or from about 10 to about 100,000, or from about 10 to about 1,000,000, or from about 10 to about 10 to about 10,000,000, or from about 100 to about 1,000, or from about 100 to about 10,000, or from about 100 to about 100,000, or from about 100 to about 1,000,000, or from about 100 to about 10,000,000, or from about 1,000 to about 10,000, or from about 1,000 to about 100,000, or from about 1,000 to about 1,000,000, or from about 1,000 to about 10,000,000, or from about 10,000 to about 100,000, or from about 10,000 to about 1,000,000, or from about 10,000 to about 10,000,000 or from about 100,000 to about 1,000,00, or 100,000 to about 10,000,000 times lower than the binding affinity of the corresponding activatable binding polypeptide.

In some embodiments, the K_{d} of the MM towards the BM is greater than the K_{d} of the BM towards the biological target. In these embodiments, the K_{d} of the MM towards the BM may be at least about 5, at least about 10, at least about 25, at least about 50, at least about 100, at least about 250, at least about 500, at least about 1,000, at least about 2,500, at least about 5,000, at least about 10,000, at least about 100,000, at least about 1,000,000, or even 10,000,000 times greater than the K_{d} of the BM towards the biological target.

Illustrative MMs include those provided as SEQ ID NOS: 85-188 (for use in an anti-CD166 activatable antibody), as well as those disclosed in WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, PCT/US2017/059740, U.S. Provisional Application Ser. Nos. 62/469,429, 62/572,467, and 62/613,358, each of which is incorporated herein by reference in its entirety.

The binding moiety may be any of a variety of polypeptides that is capable of specifically binding a desired biological target. Illustrative classes of biological targets include cell surface receptors and secreted binding proteins (e.g., growth factors, and the like), soluble enzymes, structural proteins (e.g., collagen, fibronectin, and the like), and the like. Suitable biological targets include, for example, 1-92-LFA-3, α4-integrin, α-V-integrin, α4β1-integrin, AGR2, Anti-Lewis-Y, Apelin J receptor, APRIL, B7-H4, BAFF, BTLA, C5 complement, C-242, CA9, CA19-9 (Lewis a), carbonic anhydrase 9, CD2, CD3, CD6, CD9, CD11a, CD19, CD20, CD22, CD25, CD28, CD30, CD33, CD40, CD40L, CD41, CD44, CD44v6, CD47, CD51, CD52, CD56, CD64, CD70, CD71, CD74, CD80, CD81, CD86, CD95, CD117, CD125, CD132 (IL-2RG), CD133, CD137, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA-4, CTGF, CXCL10, CXCL13, CXCR1, CXCR2, CXCR4, CYR61, DL44, DLK1, DLL4, DPP-4, DSG1, EGFR, EGFRviii, Endothelin B receptor (ETBR), ENPP3, EpCAM, EPHA2, ERBB3, F protein of RSV, FAP, FGF-2, FGF-8, FGFR1, FGFR2, FGFR3, FGFR4, Folate receptor, GAL3ST1, G-CSF, G-CSFR, GD2, GITR, GLUT1, GLUT4, GM-CSF, GM-CSFR, GP IIb/IIIa receptors, GP130, GPIIB/IIIA, GPNMB, GRP78, Her2/neu, HVEM, Hyaluronidase, ICOS, IFNα, IFNβHGF, hGH, hyaluronidase, ICOS, IFNα, IFNβ, IFNγ, IgE, IgE receptor (FceRI), IGF, IGF1R, IL1B, IL1R, IL2, IL11, IL12p40, IL-12R, IL-12Rβ1, IL13, IL13R, IL15, IL17, IL18, IL21, IL23, IL23R, IL27/IL27R (wsx1), IL29, IL-31R, IL31/IL31R, IL-2R, IL4, IL4-R, IL6, IL-6R, Insulin Receptor, Jagged Ligands, Jagged 1, Jagged 2, LAG-3, LIF-R, Lewis X, LIGHT, LRP4, LRRC26, MCSP, Mesothelin, MRP4, MUC1, Mucin-16 (MUC16, CA-125), Na/K ATPase, Neutrophil elastase, NGF, Nicastrin, Notch Receptors, Notch 1, Notch 2, Notch 3, Notch 4, NOV, OSM-R, OX-40, PAR2, PDGF-AA, PDGF-BB, PDGFRα, PDGFRβ, PD-1, PD-L1, PD-L2, Phosphatidylserine, P1GF, PSCA, PSMA, RAAG12, RAGE, SLC44A4, Sphingosine 1 Phosphate, STEAP1, STEAP2, TAG-72, TAPA1, TGFβ, TIGIT, TIM-3, TLR2, TLR6, TLR7, TLR8, TLR9, TMEM31, TNFα, TNFR, TNFRS12A, TRAIL-R1, TRAIL-R2, Transferrin, Transferrin receptor, TRK-A, TRK-B, uPAR, VAP1, VCAM-1, VEGF, VEGF-A, VEGF-B, VEGF-C, VEGF-D, VEGFR1, VEGFR2, VEGFR3, VISTA, WISP-1, WISP-2, WISP-3, and the like.

In some embodiments, the binding moiety comprises a non-antibody polypeptide, such as, for example, the soluble domain of a cell surface receptor, a secreted binding polypeptide, a soluble enzyme, a structural protein, and portions and variants thereof. As used herein, the term “non-antibody polypeptide” refers to a polypeptide that does not comprise the antigen binding domain of an antibody. Illustrative non-antibody polypeptides that are suitable for use as binding moieties in the activatable binding polypeptides employed herein include any of the biological targets listed above, as well as portions (e.g., soluble domains) and variants thereof.

In one embodiment, the activatable binding polypeptide is an activatable antibody. As used herein, the term “activatable antibody” refers to an activatable binding polypeptide in which the binding moiety comprises a full-length antibody or portion thereof. Typically, in these embodiments, the binding moiety comprises at least a portion of the antigen binding domain. The term “antigen binding domain” refers herein to the part of an immunoglobulin molecule that participates in antigen binding. The antigen binding site is formed by amino acid residues of the N-terminal variable (“V) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of an antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)); Chothia & Lesk, J. Mol. Biol. 196:901-917 (1987); Chothia, et al. Nature 342:878-883 (1989)).

Activatable antibodies may comprise, for example, one or more variable or hypervariable region of a light and/or heavy chain (V_{L} and/or V_{H}, respectively), variable fragment (Fv, Fab′ fragment, F(ab′)₂ fragments, Fab fragment, single chain antibody (scAb), single chain variable region (scFv), complementarity determining region (CDR), domain antibody (dAB), single domain heavy chain immunoglobulin of the BHH or BNAR type, single domain light chain immunoglobulins, or other polypeptide known to bind a biological target. In some embodiments, an activatable antibody comprises an immunoglobulin comprising two Fab regions and an Fc region. In some embodiments, an activatable antibody is multivalent, e.g., bivalent, trivalent, and so on. In some embodiments, the activatable antibody comprises a prodomain joined to the N-terminus of the VL domain of the antibody (or portion thereof) component of the activatable antibody (e.g., from N-terminus to C-terminus, MM-CM-VL, where each “-” refers to a direct or indirect linkage). In some embodiments, the activatable antibody comprises a prodomain joined to the N-terminus of the VH domain of the antibody (or portion thereof) component of the activatable antibody (e.g., from N-terminus to C-terminus, MM-CM-VH, where each “-” refers to a direct or indirect linkage).

Antibodies and portions thereof (including, for example, individual CDRs, as well as light and heavy chains) that are suitable for use in the activatable binding polypeptides employed herein, include, for example, any of those described in WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, PCT/US2017/059740, U.S. Provisional Application Ser. Nos. 62/469,429, 62/572,467, and 62/613,358, each of which is incorporated herein by reference in its entirety. Illustrative specific sources of antibodies or portions thereof that may be employed in the practice of the present invention include, for example, bevacizumab (VEGF), ranibizumab (VEGF), cetuximab (EGFR), panitumumab (EGFR), infliximab (TNFα), adalimumab (TNFα), natalizumab (Integrin α4), basiliximab (IL2R), eculizumab (Complement C5), efalizumab (CD11a), tositumomab (CD20), ibritumomab tiuxetan (CD20), rituximab (CD20), ocrelizumab (CD20), ofatumamab (CD20), obinutuzumab (CD20), daclizumab (CD25), brentuximab vedotin (CD30), gemtuzumab (CD33), gemtuzumab ozogamicin (CD33), alemtuzumab (CD52), abiciximab (Glycoprotein receptor IIb/IIIa), omalizumab (IgE), trastuzumab (Her2), trastuzumab emtansine (Her2), palivizumab (F protein of RSV), ipilimumab (CTLA-4), tremelimumab (CTLA-4), Hu5c8 (CD40L), pertuzumab (Her2-neu), ertumaxomab (CD3/Her2-neu), abatacept (CTLA-4), tanezumab (NGF), bavituximab (Phosphatidylserine), zalutumumab (EGFR), mapatumamab (EGFR), matuzumab (EGFR), nimotuzumab (EGFR), ICR62 (EGFR), mAB 528 (EGFR), CH806 (EGFR), MDX-447 (EGFR/CD64), edrecolomab (EpCAM), RAV12 (RAAG12), huJ591 (PSMA), etanercept (TNF-R), alefacept (1-92-LFA-3), ankinra IL-1Ra), GC1008 (TGFβ), adecatumumab (EpCAM), figitumamab (IGF1R), tocilizumab (IL-6 receptor), ustekinumab (IL-12/IL-23), denosumab (RANKL), nivolumab (PD1), pembrolizumab (PD1), pidilizumab (PD1), MEDI0680 (PD1), PDR001 (PD1), REGN2810 (PD1), BGB-A317 (PD1), BI-754091 (PD1), JNJ-63723283 (PD1), MGA012 (PD1), TSR042 (PD1), AGEN2034 (PD1), INCSHR-1210 (PD1), JS001 (PD1), durvalumab (PD-L1), atezolizumab (PD-L1), avelumab (PD-L1), FAZ053 (PD-L1), LY-3300054 (PD-L1), KNO35 (PD-L1), and the like (with biological target indicated in parentheses).

In one embodiment, the BM or AB comprises an anti-CD166 antibody or portion thereof. Illustrative anti-CD166 antibodies (or portions thereof), include, for example, those having all or a portion of a VH region of an anti-CD166 antibody (including, for example, those encoded by SEQ ID NO: 205 and SEQ ID NO: 206) and/or all or a portion of a VL region of an anti-CD166 antibody (including, for example, any of the VL domains encoded by SEQ ID NOs: 211-215. Illustrative activatable anti-CD166 antibodies include an activatable anti-CD166 antibody comprising a light chain having an amino acid sequence corresponding to any one of SEQ ID NOs:217-221, and a heavy chain corresponding to SEQ ID NO:222. Additional activatable anti-CD166 antibodies, and portions thereof, that are suitable for use in the practice of the present invention include those described in WO 2016/179285, which is incorporated herein by reference in its entirety.

### Introduction to QSP Model

As described herein with respect to certain embodiments, quantitative systems pharmacology (“QSP”) models can predict not only the distribution of intact activatable antibody species following administration to a subject, but also the distribution of various activated antibody species with one or both MMs unmasked. To this end, the QSP models account for properties specific to activatable antibodies. Examples of such properties include interactions involving a masking moieties (MM) of an activatable antibody, where the MM reduces binding affinity of the activatable antibody to its target compared to its parental antibody. Such interactions include cleavage of the cleavable moiety (CM) by a cleaving agent, resulting in release of the MM from the activatable antibody, and “breathing” of the MM due to a conformational change of the prodomain while attached to the activatable antibody, both of which can result in unmasking of the target-binding fragment of the activatable antibody. As noted, in some embodiments the MM is attached to the AB by a cleavable moiety (CM) as part of a prodomain, where CM is a polypeptide that can act as a protease substrate. In some cases, the models account for the strength of the mask, the cleavability of the substrate, and affinity of the parental antibody.

The QSP models herein consider “compartments” of subjects who are administered activatable antibodies. Typically, though not necessarily, the compartments include a target compartment (e.g., a portion of the subject's body that produces antigens targeted by the activatable antibody) and at least one non-target compartment (e.g., a portion of the subject's body, such as the subject's plasma, to which the activatable antibody is typically administered). For each compartment, the QSP models represent the local physical and chemical processes that affect the activity and/or disposition of the activatable antibody. Such processes include inter-compartmental transfer of the activatable antibody and reactions of the activatable antibody. Examples of reactions include binding to the target by activatable antibodies and activated antibodies, and reactions affecting the MM portion of the activatable antibody; e.g., cleavage of the MM from the activatable antibody and breathing of the MM while attached to the activatable antibody. Each of these processes may be compartment specific; i.e., different compartments provide different environments that affect the activity and disposition of the activatable antibody species.

In some embodiments, QSP models predicting the distribution and/or activity of an activatable antibody by considering some or all of the following factors: antibody affinity, masking strength, substrate stability, protease activity, receptor density, tumor perfusion rate, partition coefficient, and volume.

### Flow Charts

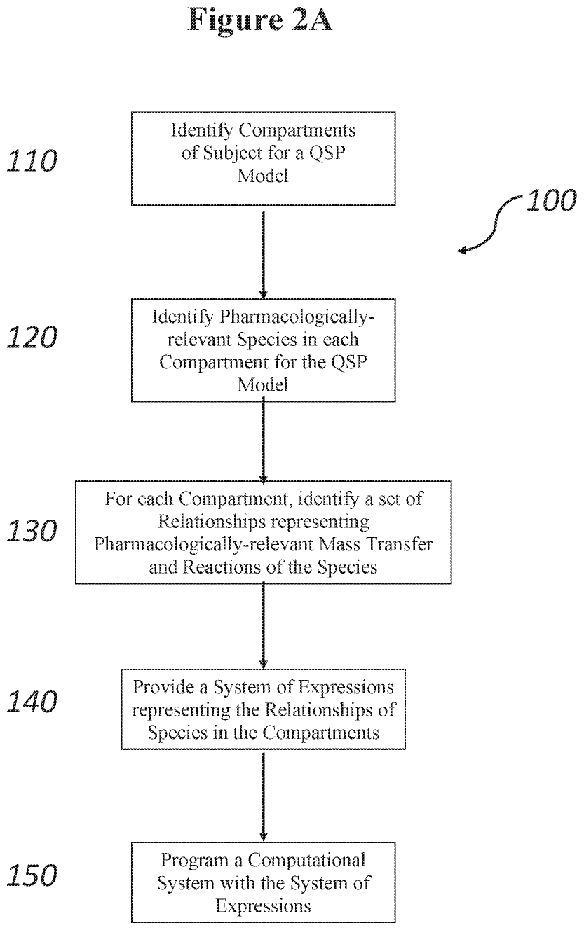

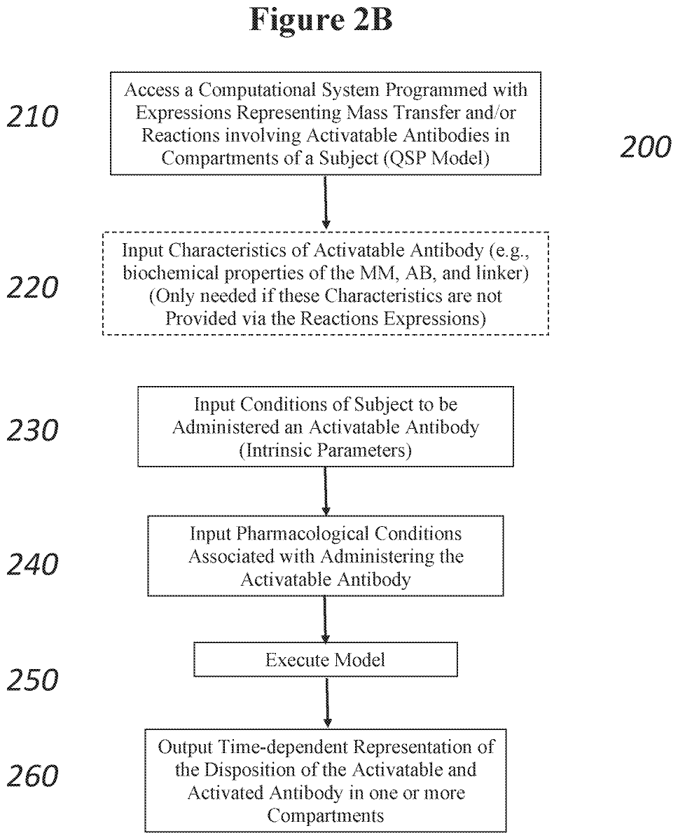

FIG. 2A presents a flow chart for an example method of generating a QSP model for activatable antibodies. The method is represented by reference numeral #100 and begins with an operation #110 in which a computer system used in generating the model receives information about compartments that will be used in the QSP model. As explained, compartments represent portions of a subject's body, and typically include at least one target compartment and at least one non-target compartment. Compartments are generally chosen because they are relevant to determining a time-dependent concentration or amount (mass or density) of activatable antibody and/or determining associated PK/PD parameters. The QSP model will use compartments to provide boundaries between regions of the subject's body where the activatable antibody is subject to different environments and consequently different reactions and/or reaction conditions. The activatable antibody can move between compartments via physical transport mechanisms such as diffusion, perfusion, and/or active transport. Further, even without such mass transport mechanisms, an activatable antibody's concentration in a compartment may change due to osmosis, degradation (extra- or intracellular), or other passive or active mechanism in which the antibody doesn't necessarily pass between compartments.

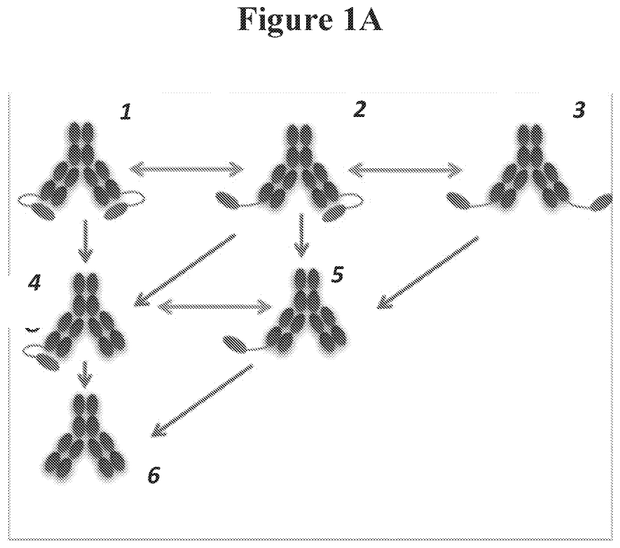

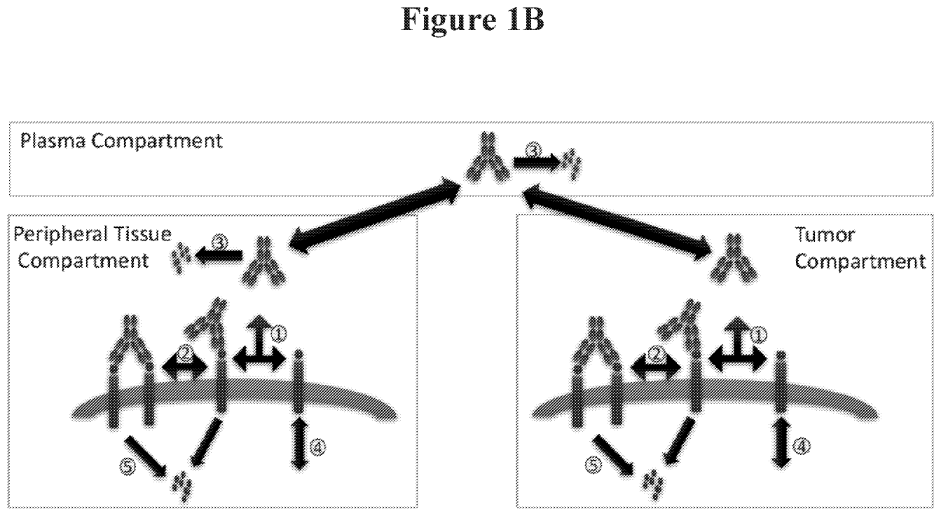

With the compartments in this exemplary embodiment defined, the system next receives identities of the pharmacologically-relevant species in each compartment. See operation #120 where the computer system receives this information. These species may be chosen because they influence a time-dependent concentration or amount of the activatable antibody. Typically, the activatable antibody is one such species. Other species include various species of activated antibody, which result from the unmasking of one or two prodomains of the activatable antibody, which can result from the cleavage of the CM by a cleaving agent and/or conformational unmasking of the MM. For example, as shown in the schematic of FIG. 1A, the activatable antibody is depicted as an intact species with two linked prodomains (items no. 1, showing ovals attached and masked to both arms as shown in the upper three configurations). This figure also schematically depicts different species of activated antibody, including an activated antibody with one conformationally unmasked prodomain (item no. 2), an activated antibody with two conformationally unmasked prodomains (item no. 3), an activated antibody with one prodomain removed, e.g., by cleavage, from the activatable antibody (item no. 4), an activated antibody with one prodomain removed, e.g., by cleavage, from the activatable antibody and one conformationally unmasked prodomain (item no. 5), and an activated antibody with two prodomains removed, e.g., by cleavage, from the activatable antibody (item no. 6).

Other pharmacologically-relevant species in the target compartment include species of the target, which is often an antigen, and the amount of the target that is bound with various of the aforementioned species of activated antibody. In some target compartments, such as when the target compartment is a tumor compartment, the pharmacologically-relevant species include increased levels and/or activity of proteases or other cleaving agents for the prodomain of the activatable antibody. The QSP model does not necessarily assume that all species or activatable or activated antibodies are present in all compartments. For example, the model may assume that no protease is present in a non-target compartment or in a peripheral or non-tumor target compartment. Operation #120 may also include specifying a concentration value for one or more species. If the concentration of a species is assumed to vary over the duration represented by the model, the concentration may be provided as an initial value. If, however, the concentration of the species is assumed not to vary over the duration, the concentration is provided as a constant; e.g., the concentration of antigen and/or protease in a target or non-target compartment may be assumed to be constant.

With the compartments and relevant species in each compartment identified, the computer system receives a set of relationships representing mass transfer and/or reactions of the species in the compartments. See operation #130. These relationships may include rate constants, equilibrium constants, concentrations of one or more species, etc. In certain embodiments, one or more of these relationships provide the rate of accumulation or depletion of a species due to a particular physical phenomenon (e.g., driven mass transfer between compartments or a reaction within a compartment). In some embodiments, one or more of the relationships is a ratio of concentrations of two or more species or a ratio of products of these species (e.g., an equilibrium constant or partition coefficient). In certain embodiments, the computer system obtains parameters such as rate constants for these relationships. Examples of sources of these parameters and methods of determining them are provided below.

With the relationships received, the computer system uses the rate constants, species concentrations, and any other components of the relationships to produce a system of expressions that can be used by the computational system to execute the QSP model. See operation #140. In certain embodiments, this operation includes organizing information from the set of relationships into, vectors, matrices, tensors, specified data structures, and/or other constructs that the computer system can use to calculate a time-dependent concentration of one or more species over a defined duration. The system of expressions is generally a computer-useable representation of the equations or other mathematics characterizing species in compartments. In certain embodiments, the system of expressions includes expressions representing one or more differential equations for one or more compartments. In some implementations, operations #130 and #140 are performed together. In other words, the computer system receives system of expressions directly, without first converting the relationships of operation #130 into the more computer-useful system of expressions.

With the system of expressions provided, the computer system programs a particular computational system with the system of expressions in a form ready for execution. See operation #150. In some cases, the computer system used to generate the QSP model is the same as the particular computational system programmed to execute the model. In other cases, the two systems are different, physically or logically. The programming of operation #150 allows the computational system to execute the QSP model when provided with appropriate initial conditions (pharmacological conditions) or other information.

Receiving instructions or data in operations #110, #120, #130 and/or #140 refers to actions of by or for a computer system that generates the QSP model. These actions may include inputting and/or storing information in memory accessible by processors responsible for programming computational system with instructions and data that comprise the QSP model. A human user may be indirectly responsible for causing a transmission of instructions and/or data to the portion of the computational system where it can be used to program the QSP model.

FIG. 2B presents a flow chart for an example method of using a QSP model to determine the disposition and/or activity of an activatable antibody administered to a subject. The method is represented by reference numeral #200 and begins with an operation #210 in which a computational system used in executing the QSP model is accessed or otherwise made available for execution. In certain embodiments the QSP model is generated using a method following the process of FIG. 2A. Regardless, the computational system is programmed with expressions representing mass transfer and/or reactions involving activatable antibodies in one or more compartments of a subject.

With the QSP model available, the computational system can receive and/or input various data and/or commands necessary to execute the model in a way that predicts activatable antibody and/or activated antibody concentration and/or PK/PD characteristics. For example, the computational system may receive and/or input properties specific for a particular activatable antibody or activated antibody. See operation #220. Examples of such properties include biochemical characteristics of activatable antibody and/or activated antibody components; e.g., target-binding characteristics of the AB, masking strength of the MM, and susceptibility of the CM to proteolytic cleaving. This information may be provided various forms such as affinity constants, cleavage rate constants, and the like.

In certain embodiments, operation #220 is not performed. This may be the case where the QSP model is designed or used for only a single type of activatable antibody, for which the biochemical properties are factored into the QSP model as generated. However, when the QSP model is generated to handle multiple types of activatable antibody, the model can be used to determine how redesign or modest changes to an activatable antibody can impact the PK/PD properties of the activatable antibody and/or activated antibody in a subject.

In an operation #230, the computational system receives or inputs conditions of a subject who is to be administered an activatable antibody. As explained elsewhere herein, such conditions are sometimes referred to as intrinsic parameters and include information such as the mass of the subject and characteristics of a tumor or other target compartment in the subject. The intrinsic parameters may be gathered by performing one or more tests on or measurements of the subject. For example, characteristics of a tumor may be gathered from a biopsy and/or tomography.

In an operation #240, the computational system receives or inputs one or more pharmacological conditions associated with administering the activatable antibody to the subject. Such pharmacological conditions are sometimes referred to as extrinsic parameters. As explained herein, such parameters concern the subject's treatment and they may include various details about how the activatable antibody is administered to the subject; e.g., doses in a treatment regimen.

With the intrinsic and extrinsic parameters available, the QSP model is ready to execute. Execution is depicted in operation #250 of FIG. 2B and involves performing various mathematical or numerical operations on the data and/or commands received via operations #230, #240, and optionally #250. The mathematical or numerical operations are performed by following instructions for, e.g., solving a system of expressions such as generated in operation #140 of FIG. 2A.

During or after execution, the computational system outputs values relevant to the distribution and/or activity of the activatable antibody and/or activated antibody in one or more compartments of the subject. See operation #260. These values may be time-dependent representations of the activatable antibody concentration and activated antibody species concentrations, or amounts thereof in one or more of the compartments (e.g., a plasma compartment, a peripheral compartment, and a target compartment). In certain embodiments, the values are PD or PK parameters such as target occupancy by various species of activated antibody in a target compartment, target occupancy by the various species of activated in a peripheral compartment, therapeutic window, target mediated drug disposition in the target compartment, target mediated drug disposition in the peripheral compartment, target mediated drug disposition in the plasma compartment, concentrations of various species of activated antibodies and/or activatable antibody in the target compartment, concentrations of various species of activated antibodies and/or activatable antibody in the plasma compartment, and concentrations of various species of activated antibodies and/or activatable antibody in the peripheral compartment.

### Species Details

### Activatable Antibody Details

As explained herein, an activatable antibody in an intact form is a species in which the prodomains are chemically linked to the antibody, and the MMs are masking the binding elements of the antibody. The intact activatable antibody has a lower binding affinity to its target compared to the parental antibody. Species of activated antibodies include fully cleaved activated antibody (both prodomains cleaved from the antibody), and partially cleaved activated antibody (one prodomain linked to the antibody and the other prodomain cleaved from the antibody). Further, an uncleaved or partially cleaved activatable or activated antibody may undergo “breathing” in which the prodomain linked to an arm of the antibody undergoes conformation changes that vary the degree to which the MM inhibits binding of the antibody to a target. As described herein, the various species of activated antibody have a reduced inhibition of binding affinity to its specific target compared to the binding affinity of the intact activatable antibody to the target.

A schematic for interactions involving species of activatable antibody and activated antibodies is provided in FIG. 1. The activatable antibody is depicted as an intact species with two linked prodomains (items no. 1, showing ovals attached and masked to both arms as shown in the upper three configurations), with subsequent successive stages of irreversible cleavage reactions. The first and second cleavage reactions generate mono-cleaved activated antibody species (item nos. 4 and 5; middle tier) and subsequently dual-cleaved, activated antibody (item no. 6; bottom left), respectively. In addition, the figure shows reversible breathing reactions that reflect mask binding to the antibody. Breathing conformations are shown for all species but the dual-cleaved parental antibody. Thus, this figure schematically depicts different species of activated antibody that an activated antibody with one conformationally unmasked prodomain (item no. 2), an activated antibody with two conformationally unmasked prodomains (item no. 3), an activated antibody with one prodomain removed, e.g., by cleavage, from the activatable antibody (item no. 4), an activated antibody with one prodomain removed, e.g., by cleavage, from the activatable antibody and one conformationally unmasked prodomain (item no. 5), and an activated antibody with two prodomains removed, e.g., by cleavage, from the activatable antibody (item no. 6).

The intact activatable antibody species with both MMs are masking the antibody binding elements binds the target with a lower binding affinity as compared to the parental antibody. The activated antibody with one prodomain removed and the other binding domain masked, and the activated antibody with only one binding element conformationally unmasked exhibit monovalent binding to target. The species on the diagonal of FIG. 1 are activatable antibodies with combinations of breathing and/or cleavage reactions that result in both arms available for bivalent binding. Reversible breathing events (represented by bidirectional arrows) and irreversible cleavage reactions (represented by unidirectional arrows) may both be captured in the model.

### Target Details

In many embodiments of the present disclosure, a target is an antigen that is specifically bound by the parental antibody of an activatable antibody and activated antibody as described herein. The term target is sometimes extended to include tissue, an organ, a tumor, etc. that disproportionately expresses the target antigen. In the context of a QSP model, a target is present in a higher quantity in a target compartment than in non-target compartments. Target compartments in the QSP model include, for example, both the tumor compartment and the peripheral compartment. In some embodiments of the model, both the tumor and peripheral compartments have the same steady state amounts of target available for binding. In some embodiments of the model, the tumor and peripheral compartments can have different steady state amounts of target available for binding, which may result from differences in synthesis of the target, differences in recycling of the target, or differences in both. In the context of an activatable antibody targeting cancer, the target is generally an antigen that is strongly associated with a tumor to be treated. In some embodiments, the target may be an antigen associated with non-cancer indications.

In certain embodiments, a QSP model accounts for target quantity in one or more compartments. The target concentration may vary from compartment-to-compartment. In certain embodiments, the target concentration in a compartment is treated as a constant. In certain embodiments, the target concentration varies with time. The QSP model may account for the target concentration in various ways. For example, an expected constant target concentration may factor into the value of a rate constant for binding with a species of activated antibody. Or, a time varying target concentration may appear explicitly in a rate expression affecting the disposition or binding of an activatable antibody species. When using a time varying target concentration, a QSP model may present a rate of expression of the target, a rate of endocytosis of the target, and/or the rate of regeneration of the within any particular compartment, often at least a target compartment. While this description has referred to a target “concentration,” the amount of target in a compartment may be reflected in other ways such as by a total amount (mass or molar amount).

### Protease Details

As explained herein, the prodomains linked to the parental antibody in an activatable antibody or certain species of activated antibody can include a CM that can serve as a substrate of a cleaving agent. In some embodiments, the cleaving agent is one or more target-associated proteases; the prodomain is then preferentially cleaved and released in the vicinity of the target relative to other locations in the target, resulting in local activation of activatable antibody at the intended site of action and, subsequently, enhanced therapeutic index in nonclinical model. A QSP model may handle the amount of cleaving agent, such as a protease, in a compartment explicitly or implicitly. If handled explicitly, the protease quantity is present as a constant or time-varying quantity in a relationship for a compartment. For example, the protease concentration may be present in a rate expression for cleaving prodomains from an activatable antibody. Alternatively, the protease concentration may impact the value of a rate constant or other parameter affecting the disposition of activatable antibody in a particular compartment.

### Compartment Details

The QSP model will define compartments to provide boundaries between regions of the subject's body where the activatable antibody and activated antibody species are subject to different environments and consequently different reactions and/or reaction conditions. For example, one compartment may include relatively high concentrations of a target (e.g., antigen) and a protease for a linker/substrate. A different compartment might contain relatively low concentrations of these species or contain them in a form in which they are less reactive (e.g., embedded in an extracellular matrix). Compartments are generally chosen for a model because they are relevant to determining a time-dependent concentration or amount (mass or density) of activatable antibody and/or determining associated PK/PD parameters.

While compartments in QSP models provide boundaries between regions of the subject's body, the models are typically designed to account for movement of activatable antibody and activated antibody species between compartments via physical transport mechanisms such as diffusion, perfusion, and/or active transport. Further, even without such mass transport mechanisms, an activatable antibody's or activated antibody's concentration in a compartment may change due to osmosis or other mechanism in which the antibody doesn't necessarily pass between compartments.

In certain embodiments, a QSP model includes at least one target compartment and at least one non-target compartment. The target compartment may have a higher concentration or amount of target compared to at least one other compartment. Or the target compartment may represent an environment containing a tumor or other deleterious feature in a subject's body; i.e., the target compartment is where the activatable antibody is intended to reduce the impact of a disease or other deleterious action. In certain embodiments, the target compartment is not the compartment with the highest concentration of target but it does contain an environment that selectively activates the activatable antibody; e.g., it contains a high concentration of a protease that cleaves the MM from the activatable antibody.

In some cases, a non-target compartment is a compartment where the activatable antibody is administered. Sometimes such compartments are referred to as “central” compartments. In one example, the non-target compartment is a plasma compartment.

In certain embodiments, the QSP model employs three or more compartments. There may be a target compartment and two or more non-target compartments. For example, there may be a central compartment, which may be a plasma compartment, and a “peripheral” compartment that represents one or more non-target organs or tissues in the subject. A peripheral compartment may or may not be physiological; e.g., it may contain multiple non-target organs/tissues, or even a subset of one organ or tissue.

In some cases, a compartment does not directly map to a real biological system. As an example, a compartment maps to a placeholder for some PD/PK related activity or disposition of the activable antibody or activated antibody species. The placeholder may be of unknown location or function in an organism.

An example of a compartmental arrangement for a QSP model is depicted in FIG. 2(b). The depicted arrangement includes plasma, peripheral, and tumor compartments. Concurrently, all six forms/species of an activatable antibody distribute to the plasma, peripheral, and tumor compartments. In the peripheral and tumor compartments, a subset of activatable or activated antibodies may engage in monovalent (1) or bivalent (2) binding, depending upon the number of breathing or cleaved binding sites, respectively. Circulating activatable and activated antibody and unbound activatable and activated antibody in the peripheral compartment (3), and internalized activatable and activated antibody (5) may be all be eliminated. The amount of target (4) that is available for binding by the activatable and activated antibody is determined by the expression and internalization rates of the targets in the compartments.

### Reactions and Transport Details

As explained, a QSP model uses various relationships and other details for mass transfer and reactions affecting the concentration of activatable and activated antibody species in each of the compartments. For example, a QSP model may be based on mechanisms of activatable and activated antibody breathing, cleavage, plasma elimination, tissue and tumor biodistribution, receptor binding, and/or receptor-drug complex endocytosis. In certain embodiments, reactions are modeled with 0^{th}, 1^{st}, and 2^{nd} order mass action relationships.

In certain embodiments, the activatable antibody is depicted first as an intact moiety with two prodomains, with subsequent successive stages of irreversible cleavage reactions characterized by a pseudo-first order rate constant k_{cleave} (s⁻¹) which captures both the rate of substrate proteolysis and protease concentration. The first and second cleavage reactions generate the mono-cleaved activated antibody species and subsequently the dual-cleaved, parental activated antibody, respectively. In addition, in certain embodiments, reversible breathing reactions that reflect mask binding to the parental antibody are included for all but the dual-cleaved parental activated antibody. In some implementations, breathing reactions are expressed in terms of a ratio (Kₘₐₛₖ) of first-order rate constants for mask closing (k_{close}, s⁻¹) and opening (kₒₚₑₙ, s⁻¹). The activatable antibody species with both masks closed may be modeled to so that it does not bind target, but both the mono-cleaved activated antibody with one mask closed and intact activated antibody with one mask open may be treated as exhibiting monovalent binding to target. In certain embodiments, the species of activatable and activated antibody are allowed to distribute to plasma, peripheral, and tumor compartments as depicted in FIG. 1(b). In certain embodiments, all free activatable and activated antibody species are assumed to be eliminated from the plasma compartment at the same first-order rate constant kₑₗ (s⁻¹). The model may allow activatable and activated antibody species in the plasma compartment to equilibrate with the peripheral compartment with inter-compartment transport rate constants k₁₂ (s⁻¹) and k₂₁ (s⁻¹), respectively. The model may also allow plasma activatable and activated antibody species to further distribute to the tumor compartment with inter-compartment transport rate constants k₁₃ (s⁻¹) and k₃₁ (s⁻¹), respectively. In some implementations, the constants k₁₃ (s⁻¹) and k₃₁ (s⁻¹) are derived from a plasma to tumor steady state activatable and activated antibody concentration ratio (partition coefficient, p) and a plasma to tumor perfusion rate (Q (s⁻¹)). Within both peripheral and tumor compartments, monovalent and bivalent activated antibody species may bind target with forward rates kₒₙ₁ (nM⁻¹ s⁻¹), and both kₒₙ₁ and kₒₙ₂ (nM⁻¹ s⁻¹), respectively, and reverse rate constant k_{off1} (s⁻¹). In certain embodiments, kₒₙ₂ is estimated under the assumption that avidity is not influential (i.e., kₒₙ₁=kₒₙ₂). Target expression in both the periphery and tumor may be governed by target synthesis and endocytosis rate constants k_{synR} (nmol s⁻¹) and k_{endo} (s⁻¹), respectively. In some implementations, the constants k₁₃ (s⁻¹) and k₃₁ (s⁻¹) that described the plasma to tumor ratio P_{T} and tumor perfusion rate Q_{T} (s⁻¹) were derived as follows:

k₁₃=Q_{T}P_{T}/(P_{T}+V₁/V₂)

k₃₁=Q_{T}/(1+P_{T}V₂/V₁)

Examples of relationships that may be used in QSP models follow. In certain embodiments, a QSP model employs any one or more of these relationships. In certain embodiments, a QSP model employs any two or more of these relationships.

### Rate of Producing Cleaved Activated Antibody (AA) (First Order):

d[AA_{cleaved}]/dt=k_{cleave}[AA_{uncleaved}]

There may, of course, be two of these expressions one for producing partially cleaved AA and a second one for producing fully cleaved AA.

### Equilibrium in “Breathing” Conformations (First Order):

Kₘₐₛₖ=[k_{close}/kₒₚₑₙ]

Elimination from Plasma (or Other Compartment) (First Order):

d[AA]/dt=kₑₗ[AA]

In any given compartment, there may be one of these expressions for each of three species of AA, uncleaved, partially cleaved, and fully cleaved.
Mass Transfer from any One Compartment to a Different Compartment (First Order):

d[AA]/dt=k₁₂[AA]

In this expression, 1 and 2 represent two different compartments. There may be separate mass transfer expressions for each of the three AA species.

### AA Binding to Target (Second Order):

d[AA]/dt=−kₒₙ[AA][Antigen]

In each of the target and peripheral compartments, there may be separate expressions for the monovalent (partially cleaved) and bivalent (fully cleaved) AAs.
Bound AA Release from Target (First Order):

d[AA]/dt=k_{off}[AA_{bound}]

### Target Expression (Zero Order):

d[Antigen]/dt=k_{synR}

### Target Endocytosis (First Order):

d[Antigen]/dt=−k_{endo}[Antigen]

### Expressions for Representation QSP Model in a Computational System

As discussed, the QSP model of the present disclosure executes instructions representing mathematical expressions characterizing one or more of the species in each compartment. The mathematical representation is provided as a set of expressions of the relationships and quantities for the species in each compartment. In some implementations, each compartment has one or more separate mathematical expressions, with one for each species under consideration in the compartment. The expressions correspond to the governing relationships, such as the reaction and transfer phenomena described above. The mathematical representation may include all information sufficient for representing or predicting (through computation) a time varying concentration of the component of interest in the compartment of interest.

For example, a plasma compartment may have mathematical expressions representing the reactions and transport of each of species of activatable and activated antibody (uncleaved, partially cleaved, and fully cleaved). Likewise, a target compartment may have mathematical expressions representing the reactions and transport of a target, a cleaving agent such as a protease, as well as each of the species of activatable and activated antibody. Likewise, a peripheral compartment may have mathematical expressions representing the reactions and transport of each of species of activatable and activated antibody. Stated another way, if uncleaved activatable and activated antibody, partially cleaved activated antibody, fully cleaved activated antibody, and target are to be modelled in the target compartment, at least four separate mathematical expressions are provided for the target compartment, one for each of the species in the target compartment.

In certain embodiments, the mathematical expressions are differential equations providing time-dependent representations of the species of interest in a particular compartment. The differential equations may include vectors and/or matrixes of rate constants or other parameters affecting the concentration or amount of the species of interest in the particular compartment. In some cases, the differential equation is represented by the following equation:

$$\frac{d x}{d t} = k + A x + B ( x ⊗ x )$$

In this expression, x is a concentration or amount of species, t is time, k is a vector of zeroith order rate constants, A is an n by n matrix of first order rate constants, and B is a n by n matrix of second order rate constants.

In these and other implementations, the individual differential equations and or other mathematical representations of the components' concentrations in the individual compartments are solved simultaneously, typically by numerical means, to provide time-dependent values of each of the components in each of the compartments. In one example, QSP models are implemented using KroneckerBio version 0.4, which is open source software and is maintained at the following website: github.com/kroneckerbio. As a further example, simulation runs, parameter estimation, and parameter scans are performed using MATLAB version 2015b (Mathworks, Natick Mass.).

To solve for the time-dependent concentrations, not only are the rate constants and other information about the reactions and transport required (e.g., via differential equations programmed into a computational system), but a set of subject-specific parameters are also required. These include intrinsic parameters and extrinsic parameters. Intrinsic parameters are parameters specific to the subject and outside the control of a physician or clinician treating the subject. Extrinsic parameters are parameters under the control of the physician or clinician. Examples of intrinsic parameters include the mass of the subject, and characteristics of the compartments that are specific to the subject. For a target compartment, examples of relevant parameters include the size of the tumor and the concentration or rate of generation of target. Examples of extrinsic parameters include the dose of an activatable antibody administered, frequency of dose of the activatable antibody, other medicaments administered concurrently with the activatable antibody, and the like.

Therefore, in some implementations, a computational system is programmed with mathematical expressions representing the activity and/or disposition of activatable antibody species, and possibly other species, on a compartment-by-compartment basis. The computational system programmed in this manner can receive as inputs various intrinsic and/or extrinsic parameters and then execute a QSP model to solve for the time-varying concentrations of activatable antibody species and possibly other species. With the time-varying concentrations of these various components in the various compartments, various pharmacokinetic and pharmacodynamics parameters can be generated. Examples of these parameters include the following: target occupancy by the activatable antibody in a target compartment, target occupancy by the activated and/or activatable antibody in a peripheral compartment, therapeutic window, target mediated drug disposition in the target compartment, target mediated drug disposition in the peripheral compartment, target mediated drug disposition in the plasma compartment, concentrations activated and/or activatable antibody in the target compartment, concentrations activated and/or activatable antibody in the plasma compartment, and concentrations activated and/or activatable antibody in the peripheral compartment.

### Obtaining Rate Constants and Other Parameters Required for Programming the QSP Model

Rate constants and other parameters programmed into a QSP model (e.g., included in ordinary differential equations for numerical solution) can be obtained from various sources including literature references and calibration by experimentation. Calibration may be conducted in vitro or in vivo.

In certain embodiments, determining a mass transfer rate constant for elimination or inter-compartment transport is accomplished by evaluating, at least, time-varying values of concentration of an activatable antibody in the samples taken from the one or more test subjects. Similarly, in certain embodiments, determining reaction rate constants for antibody binding, cleaving, etc. is accomplished by evaluating, at least, time-varying values of concentration of the activatable antibody in the samples taken from the one or more test subjects. These determinations may be made separately for activated and/or activatable antibodies.

In some embodiments, determining a rate constant or other parameter characterizing mass transfer or a reaction of an activatable antibody and/or activated antibody involves applying an objective function to evaluate time-varying values of concentration of the activatable antibody and/or the activated antibody in samples taken from the one or more test subjects. In some implementations, the objective function is a log likelihood function.

### Context for Disclosed Computational Embodiments

Certain embodiments disclosed herein relate to systems for generating and/or using QSP models. Certain embodiments disclosed herein relate to methods for generating and/or using a QSP model implemented on such systems. A system for generating a QSP model may be configured to analyze data for calibrating the expressions or relationships used to represent activity and disposition of activatable antibodies in a subject. In such calibration, the system may determine rate constants or other parameter values charactering activatable antibodies in the subject. A system for generating a QSP model may also be configured to receive data and instructions such as program code representing physical processes in one or more compartments of the subject. In this manner, a QSP model is generated or programmed on such system. A programmed system for using a QSP model may be configured to (i) receive input such as pharmacological conditions characterizing a subject and (ii) execute instructions that determine the disposition and/or activity of an activatable antibody in one or more compartments of the subject. To this end, the system may calculate time-dependent concentrations of the activatable antibody in the one or more compartments.

Many types of computing systems having any of various computer architectures may be employed as the disclosed systems for implementing QSP models and algorithms for generating and/or calibrating such models. For example, the systems may include software components executing on one or more general purpose processors or specially designed processors such as programmable logic devices (e.g., Field Programmable Gate Arrays (FPGAs)). Further, the systems may be implemented on a single device or distributed across multiple devices. The functions of the computational elements may be merged into one another or further split into multiple sub-modules.

In some embodiments, code executed during generation or execution of a QSP model on an appropriately programmed system can be embodied in the form of software elements which can be stored in a nonvolatile storage medium (such as optical disk, flash storage device, mobile hard disk, etc.), including a number of instructions for making a computer device (such as personal computers, servers, network equipment, etc.).

At one level a software element is implemented as a set of commands prepared by the programmer/developer. However, the module software that can be executed by the computer hardware is executable code committed to memory using “machine codes” selected from the specific machine language instruction set, or “native instructions,” designed into the hardware processor. The machine language instruction set, or native instruction set, is known to, and essentially built into, the hardware processor(s). This is the “language” by which the system and application software communicates with the hardware processors. Each native instruction is a discrete code that is recognized by the processing architecture and that can specify particular registers for arithmetic, addressing, or control functions; particular memory locations or offsets; and particular addressing modes used to interpret operands. More complex operations are built up by combining these simple native instructions, which are executed sequentially, or as otherwise directed by control flow instructions.

The inter-relationship between the executable software instructions and the hardware processor is structural. In other words, the instructions per se are a series of symbols or numeric values. They do not intrinsically convey any information. It is the processor, which by design was preconfigured to interpret the symbols/numeric values, which imparts meaning to the instructions.

The models used herein may be configured to execute on a single machine at a single location, on multiple machines at a single location, or on multiple machines at multiple locations. When multiple machines are employed, the individual machines may be tailored for their particular tasks. For example, operations requiring large blocks of code and/or significant processing capacity may be implemented on large and/or stationary machines. Such operations may be implemented on hardware remote from the site where a sample is acquired or where data is input; e.g., on a server or server farm connected by a network to a field device that captures the sample image. Less computationally intensive operations may be implemented on a portable or mobile device used on site for clinical evaluation.

In addition, certain embodiments relate to tangible and/or non-transitory computer readable media or computer program products that include program instructions and/or data (including data structures) for performing various computer-implemented operations. Examples of computer-readable media include, but are not limited to, semiconductor memory devices, phase-change devices, magnetic media such as disk drives, magnetic tape, optical media such as CDs, magneto-optical media, and hardware devices that are specially configured to store and perform program instructions, such as read-only memory devices (ROM) and random access memory (RAM). The computer readable media may be directly controlled by an end user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In various embodiments, the data or information employed in the disclosed methods and apparatus is provided in an electronic format. Such data or information may include pharmacological conditions associated with administering activatable antibodies to a subject, intrinsic characteristics of a subject, model parameters such as rate constants, PK/PD results, and the like. As used herein, data or other information provided in electronic format is available for storage on a machine and transmission between machines. Conventionally, data in electronic format is provided digitally and may be stored as bits and/or bytes in various data structures, lists, databases, etc. The data may be embodied electronically, optically, etc.

In certain embodiments, a QSP model can each be viewed as a form of application software that interfaces with a user and with system software. System software typically interfaces with computer hardware and associated memory. In certain embodiments, the system software includes operating system software and/or firmware, as well as any middleware and drivers installed in the system. The system software provides basic non-task-specific functions of the computer. In contrast, the modules and other application software are used to accomplish specific tasks. Each native instruction for a module is stored in a memory device and is represented by a numeric value.

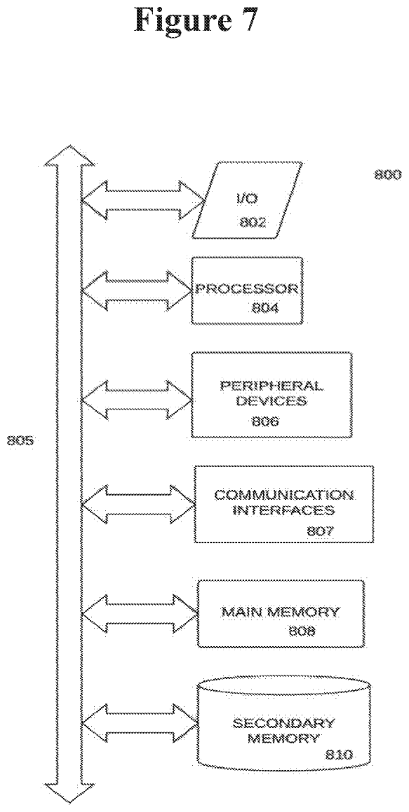

An example computer system 800 is depicted in FIG. 7. As shown, computer system 800 includes an input/output subsystem 802, which may implement an interface for interacting with human users and/or other computer systems depending upon the application. Embodiments of the invention may be implemented in program code on system 800 with I/O subsystem 802 used to receive input program statements and/or data from a human user (e.g., via a GUI or keyboard) and to display them back to the user. The I/O subsystem 802 may include, e.g., a keyboard, mouse, graphical user interface, touchscreen, or other interfaces for input, and, e.g., an LED or other flat screen display, or other interfaces for output. Other elements of embodiments of the disclosure, such as the order placement engine 208, may be implemented with a computer system like that of computer system 800, perhaps, however, without I/O.

Program code may be stored in non-transitory media such as persistent storage 810 or memory 808 or both. One or more processors 804 reads program code from one or more non-transitory media and executes the code to enable the computer system to accomplish the methods performed by the embodiments herein, such as those involved with generating or using a QSP model as described herein. Those skilled in the art will understand that the processor may accept source code, such as statements for executing training and/or modelling operations, and interpret or compile the source code into machine code that is understandable at the hardware gate level of the processor. A bus couples the I/O subsystem 802, the processor 804, peripheral devices 806, memory 808, and persistent storage 810.

### RESULTS/EXAMPLES

### Example 1: Pharmacokinetic (PK) Studies of Anti-CD166 Activatable Antibodies (AA) in Cynomolgus Monkeys

In this exemplary study, anti-CD166 antibody test articles were administered to cynomolgus monkey test subjects and exemplary pharmacokinetic (PK) data was generated. CD166 is also known as cluster of differentiation 166, activated leukocyte cell adhesion molecule (ALCAM), and/or MEMD. CD166 is an example of an attractive target for cancer therapy, as it is highly and homogenously expressed in many tumor types but whose normal tissue expression would be problematic for a traditional mAb. Accordingly, the modification of anti-CD166 antibodies to anti-CD166 activatable antibodies can provide benefits with respect to safety and efficacy in patients.

These exemplary data were used to inform the QSP model of the present disclosure of anti-CD166 activatable antibodies with differing substrates (designated as S1 and S2, respectively) and masks (designated M1 and M2, respectively) at both the molecular species level and the compartment level.

In this study, the parental anti-CD166 antibody is designated CD166-mAb(0,0), having a light chain with the variable light chain domain (VL) of SEQ ID NO: 207 and a heavy chain with the variable heavy chain domain (VH) of SEQ ID NO: 206. The anti-CD166 activatable antibodies are designated as CD166-AA(M1,S1) having a light chain with the VL of SEQ ID NO: 212 and a heavy chain with the VH of SEQ ID NO: 206, CD166-AA(M1,S2) having a light chain with the VL of SEQ ID NO: 214 and a heavy chain with the VH of SEQ ID NO: 206, CD166-AA(M2,S1) having a light chain with the VL of SEQ ID NO: 211 and a heavy chain with the VH of SEQ ID NO: 206, and CD166-AA(M2,S2) having a light chain with the VL of SEQ ID NO: 213 and a heavy chain with the VH of SEQ ID NO: 206. The M1 and M2 refer to different masking moieties (MM) and Si and S2 refer to different cleavable moieties (CM). A sixth test article, designated CD166-AA(M2,0) is an anti-CD166 activatable antibody with MM of M1 but lacking a cleavable moiety (a light chain with the VL of SEQ ID NO: 215 and a heavy chain with the VH of SEQ ID NO: 206.

Each of the six test articles were administered to two individual cynomolgus monkeys (1 male, 1 female) via slow bolus intravenous injection on day 1. The dose levels of each test article included 3, 5, and 10 mg/kg. Levels of the anti-CD166 test articles in the subjects' sera were measured at various time points up to 21 days after administration. Activatable antibody and antibody concentrations in the cynomolgus monkey lithium heparin plasma samples from PK studies were determined using sandwich-based colorimetric ELISA methods. The ELISA methods utilized a goat anti-human IgG (H+L) to serve as the capture antibody. Test article within the standards, quality control (QC) samples, and study samples were captured by the immobilized goat anti-human IgG (H+L). Horseradish peroxidase (HRP)-labeled goat anti-human IgG (H+L) was used for detection of the captured anti-CD166 Probody in conjunction with the detection reagent 3,3′,5,5′-tetramethylbenzidine (TMB).

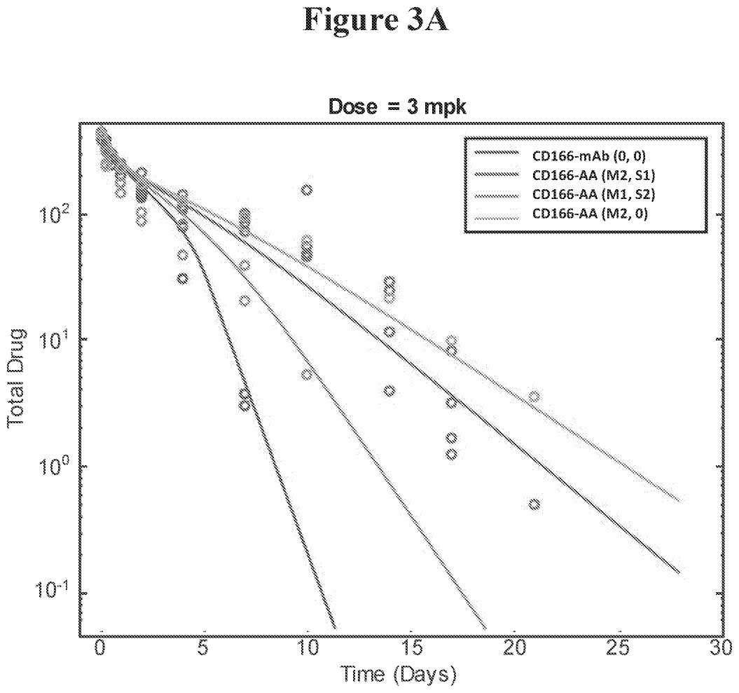

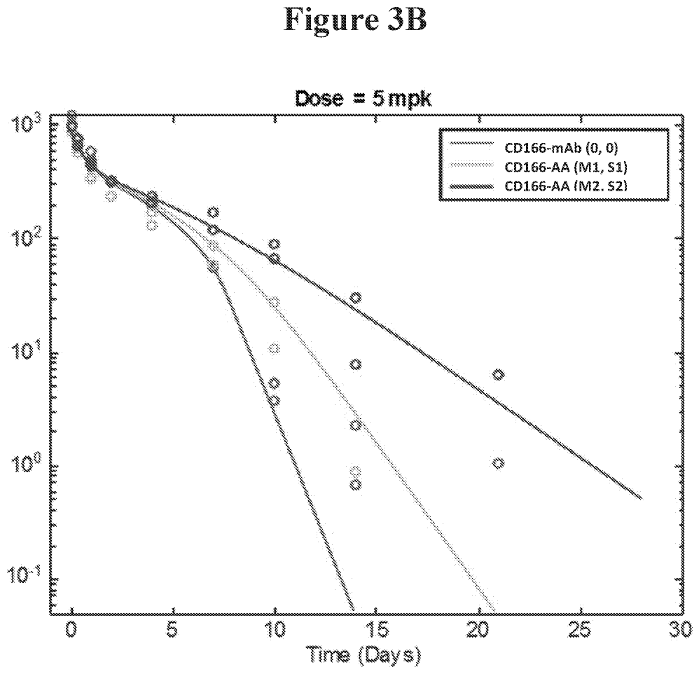

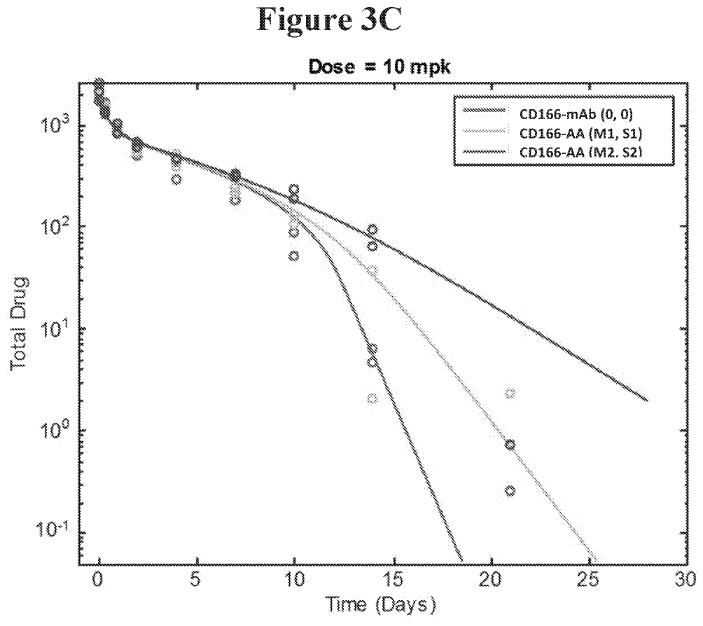

As shown in FIGS. 3A, 3B, and 3C, the observed PK in cynomolgus monkeys of the indicated test articles are shown at the indicated time points after administration. FIGS. 3A, 3B, and 3C show the PK (total amount of test article) of the 3 mg/kg, 5 mg/kg, and 10 mg/kg dosages, respectively, as hollow points.

A QSP model of the present disclosure was developed based on known mechanisms of activatable antibody breathing, cleavage, first order plasma elimination, tissue and tumor bio-distribution, receptor binding, and receptor and receptor-drug complex endocytosis. All reactions are modeled with 0^{th}, 1^{st}, and 2^{nd} order mass action reactions.

All models were implemented using KroneckerBio version 0.4. Simulation runs, parameter estimation, and parameter scans were performed using MATLAB version 2015b (Mathworks, Natick Mass.). KroneckerBio is open source software and is maintained at https://github.com/kroneckerbio. The version of KroneckerBio used for these simulations was tested prior to use and is archived by Applied BioMath (Lincoln, Mass.). Kroneker describes the systems as a system of ordinary differential equations with the following form:

$$\frac{d x}{d t} = k + A x + B ( x ⊗ x )$$

where k is a vector of 0th order rate constants, A is an n by n matrix of 1st order rate constants, and B is a n by n matrix of second order rate constants. The values of these matricies are provided as supplemental material.

The QSP model of the present disclosure was calibrated to each of the PK datasets for each of the different activatable antibody molecules. The estimation was done simultaneously for all six molecules, assuming that the physiologic model parameters (plasma volume, target expression, etc.) were constant across molecules, as well as the parameters that describe the common molecular features (mono-valent binding affinity of the open or cleaved prosody, first order elimination rate, central to peripheral distribution rates). The parameters that describe the differences between the molecules (mask strength, and cleavage rate) were simultaneously estimated using a Bayesian approach with a Gaussian log likelihood function. In brief, simulations were run over a range of parameter values for Kₘₐₛₖ and k_{cleave}. Simulations were compared to the data and a log likelihood function was computed. The exemplary parameters used or determined in the QSP model of the present disclosure are shown in Table 1.

TABLE 1
Exemplary Parameters in Monkey QSP Model

| Parameter | Value | | Notes | Reference |
|---|---|---|---|---|
| Body weight (kg) | 2.6 | kg | Fixed based on its typical value in literature | (1) |
| Central | 0.1 | L | Fixed based on its typical value in literature | (2) |
| Compartment | | | | |
| volume (Vc); | | | | |
| Plasma volume | | | | |
| (Vp) | | | | |
| kₑₗᵢₘ (s⁻¹) | 1.0e−6 | | First-order elimination rate constant of free | |
| | | | species from plasma compartment | |
| k_{endo} (s⁻¹) | 1.0e−4 | | Target endocytosis rate constant in peripheral | (3) |
| | | | compartment. | |
| k_{synR} (nmol s⁻¹) - | 5.6e−5 | | Target synthesis rate constant in peripheral | (3) |
| peripheral | | | compartment. | |
| k₁₂ (s⁻¹) | 1.0e−5 | | Inter-compartment transport rate constant | |
| | | | from plasma to peripheral compartments. | |
| k₂₁ (s⁻¹) | 1.1e−5 | | Inter-compartment transport rate constant | |
| | | | from peripheral to plasma compartments. | |
| kₒₙ₁ (nM⁻¹ s⁻¹) | 1e−3 | | Forward rate of 1^{st} binding of mono- or | |
| | | | bivalent activated antibody to target. | |
| kₒₙ₂ (nM⁻¹ s⁻¹) | 2e−3 | | Forward rate of 2^{nd} binding of bivalent | |
| | | | activated antibody to target. | |
| k_{off1} (s⁻¹) | 1e−3 | | Reverse rate constant of activated antibody | |
| | | | release of bound target. | |
| k_{close}/kₒₚₑₙ (Kₘₐₛₖ | 57 | (M1) | Ratio of MM opening (kₒₚₑₙ) and closing | |
| ratio) | 220 | (M2) | (k_{close}) first-order rate constants. | |

| | | |
|---|---|---|
| k_{cleave} (s⁻¹) (CM) | <3e−7 | Pseudo-first order rate constant of cleavage of |
| | | the CM. |

### References for Tables 1 and 2:

(1) Dong J Q, Salinger D H, Endres C J, Gibbs J P, Hsu C P, Stouch B J, et al. “Quantitative prediction of human pharmacokinetics for monoclonal antibodies: retrospective analysis of monkey as a single species for first-in-human prediction.” Clin Pharmacokinet. 2011; 50(2):131-42.
(2) Davies B, Morris T. “Physiological parameters in laboratory animals and humans.” Pharm Res. 1993; 10(7):1093-5.
(3) Lauffenburger D A, Linderman J J. Receptors models for binding, trafficking, and signaling. 1993.

The QSP model of the present disclosure was next calibrated to fit the observed cynomolgus monkey PK data. Parameter scans were conducted to compute the likelihood of Kₘₐₛₖ ratio and k_{cleave} given the data for each molecule; parameters other than Kₘₐₛₖ and k_{cleave} were set to the values of Table 1 The marginal probabilities for mask M2 produced an isolated minima; the marginal probabilities for mask M1 produced a one-sided minima which suggests it was indistinguishable from a non-binding molecule. For the parental antibody (CD166-mAb (0,0) with no mask group), the marginal probability suggested that the mask strength had to be very low which is consistent with the molecule not having a masking group. For k_{cleave}, the marginal probabilities for S1 and S2 substrates were indistinguishable from an uncleavable substrate. Likewise the k_{cleave} estimated for the parental antibody was a one-sided distribution with only very high rates being likely, consistent with the parental antibody lacking a cleavable substrate. While a unique k_{cleave} could not be estimated for the cleavable substrates, the inverse of maximum likelihood was bounded for estimates of L_{cleave}<3e⁻⁷ s⁻¹.

FIGS. 3A, 3B, and 3C suggest an adequate fit of the QSP model of the present disclosure to observed monkey PK data across the dose levels, mask strengths, and substrates entered into the evaluation. Most notably, across all dose levels, observed data exhibited the evidence of target-mediated drug disposition (TMDD) for the parental CD166-mAb (0,0) which was captured by the QSP model. At the 3 mg/kg dose level, observed data suggested decreased importance of TMDD in the disposition of the CD166 activatable antibodies overall, with evidence of TMDD minimized both for CD166 activatable antibodies with noncleavable substrate (i.e., CD166-AA (M2, 0)) and highest mask strength (CD166-AA (M2,S1) and CD166-AA (M2,S2)). These overall trends were again evident at the 5 mg/kg and 10 mg/kg dose levels where the contribution of TMDD was lessened for CD166 activatable antibodies of successively higher mask strengths (i.e. the masking strength of M2>M1); with k_{cleave} held fixed to the upper bound of 3e⁻⁷ s⁻¹, varying Kₘₐₛₖ alone by mask was sufficient for the QSP model of the present disclosure to capture trends overall.

### Example 2: Pharmacokinetic (PK) QSP Modeling of Anti-CD166 Activatable Antibodies (AA) in Humans

Following calibration against monkey PK data, the QSP model of the present disclosure was used to project human PK under a series of assumed values for the drug (e.g. Kₘₐₛₖ) and the tumor (e.g. Q and R_{T}). Fold-increases in k_{cleave} for the tumor relative to the systemic compartment was also varied to implicitly capture the effect of both drug (substrate stability) and tumor (protease activity) properties on PK. As described above in Table 1, elementary rate constants (e.g., k_{endo}, kₒₙ₁, kₒₙ₂, k_{off}) and normal tissue receptor concentration were assumed equal between monkey and human; likewise, k_{cleave} was assumed to have an upper bound of 3e⁻⁷ s⁻¹. Pharmacokinetic parameters k₁₂, k₂₁, and kₑₗᵢₘ (s⁻¹) were scaled using allometry. These and other parameter values used in human PK projection are summarized in Table 2

TABLE 2
Exemplary Parameters in Monkey QSP Model

| Parameter | Value | | Notes | Reference |
|---|---|---|---|---|
| Body weight | 2.6 | kg | Fixed based on its typical value in literature | |
| Plasma Volume | 2.6 | L | Fixed based on its typical value in literature | |
| Tumor Volume | 0.01 | L | In the range of breast tumor ER/PR+ | (4), (5) |
| kₑₗᵢₘ (s⁻¹) | 6.3e−7 | | First-order elimination rate constant of free | |
| | | | species from plasma compartment. Allometric | |
| | | | scaling | |
| k_{endo} (s⁻¹) | 1.0e−4 | | Target endocytosis rate constant in peripheral | (6) |
| | | | compartment. Same as cynomolgus monkey | |
| k_{synR} (nmol s⁻¹) - | 2.3e−3 | | Target synthesis rate constant in peripheral | |
| peripheral | | | compartment. Obtained by k_{synR} = k_{endo}*R_{T}, | |
| | | | and R_{T} is scaled by compartment volume | |
| k_{synR} (nmol s⁻¹) - | 2.7e−4 | | Target synthesis rate constant in tumor | |
| tumor | | | compartment. Obtained by k_{synR} = k_{endo}*R_{T} | |
| k₁₂ (s⁻¹) | 4.4e−6 | | Inter-compartment transport rate constant | |
| | | | from plasma to peripheral compartments. | |
| | | | Allometric scaling | |
| k₂₁ (s⁻¹) | 4.8e−6 | | Inter-compartment transport rate constant | (1) |
| | | | from plasma to peripheral compartments. | |
| | | | Allometric scaling | |
| k₁₃ (s⁻¹) | 4.4e−6 | | Inter-compartment transport rate constant | |
| K₃₁ (s⁻¹) | 8.4e−9 | | from plasma to tumor compartments (k₁₃ (s⁻¹)) | |
| | | | or from tumor to plasma compartments (k₃₁ (s⁻¹)). | |
| | | | Depends on partition coefficient which is | |
| | | | varying in the model with | |
| | | | nominal value = 0.5 | |
| kₒₙ₁ (nM⁻¹ s⁻¹) | 1e−3 | | Forward rate of 1^{st} binding of mono- or | |
| | | | bivalent activated antibody to target. Same as | |
| | | | cynomolgus monkey. | |
| kₒₙ₂ (nM⁻¹ s⁻¹) | 2e−3 | | Forward rate of 2^{nd} binding of bivalent | |
| | | | activated antibody to target. Same as | |
| | | | cynomolgus monkey.. | |
| k_{off1} (s⁻¹) | 1e−3 | | Reverse rate constant of activated antibody | |
| | | | release of bound target. Same as cynomolgus | |
| | | | monkey.. | |
| k_{close}/kₒₚₑₙ (Kₘₐₛₖ | 57 | (M1) | Ratio of MM opening (kₒₚₑₙ) and closing | |
| ratio) | 220 | (M2) | (k_{close}) first-order rate constants. Same as | |
| | | | cynomolgus monkey. | |

| | | |
|---|---|---|
| k_{cleave} (s⁻¹) (CM) | <3e−7 | Pseudo-first order rate constant of cleavage of |
| | | the CM. Same as cynomolgus monkey for |
| | | central and peripheral compartments and ×10 |
| | | for tumor compartment |

### References for Table 2:

(4) Parise C A, Bauer K R, Brown M M, Caggiano V. “Breast cancer subtypes as defined by the estrogen receptor (E R), progesterone receptor (P R), and the human epidermal growth factor receptor 2 (HER2) among women with invasive breast cancer in California, 1999-2004”. Breast J. 2009; 15(6):593-602.
(5) Ryu E B, Chang J M, Seo M, Kim S A, Lim J H, Moon W K. “Tumour volume doubling time of molecular breast cancer subtypes assessed by serial breast ultrasound”. Eur Radiol. 2014; 24(9):2227-35.
(6) Deng R, Iyer S, Theil F P, Mortensen D L, Fielder P J, Prabhu S. “Projecting human pharmacokinetics of therapeutic antibodies from nonclinical data: what have we learned?” MAbs. 2011; 3(1):61-6.

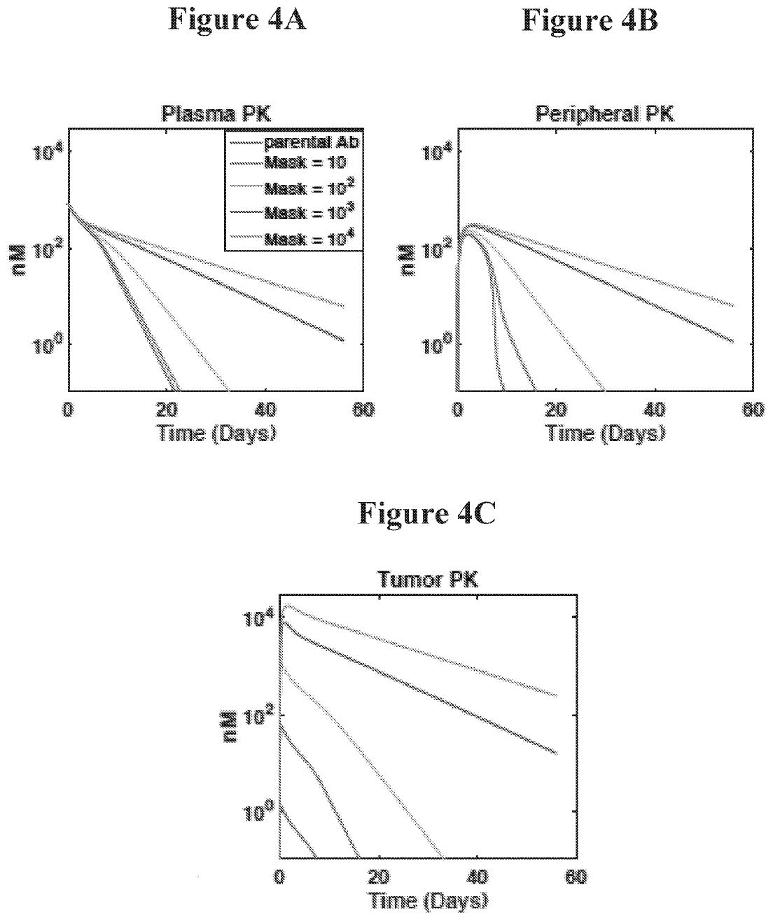

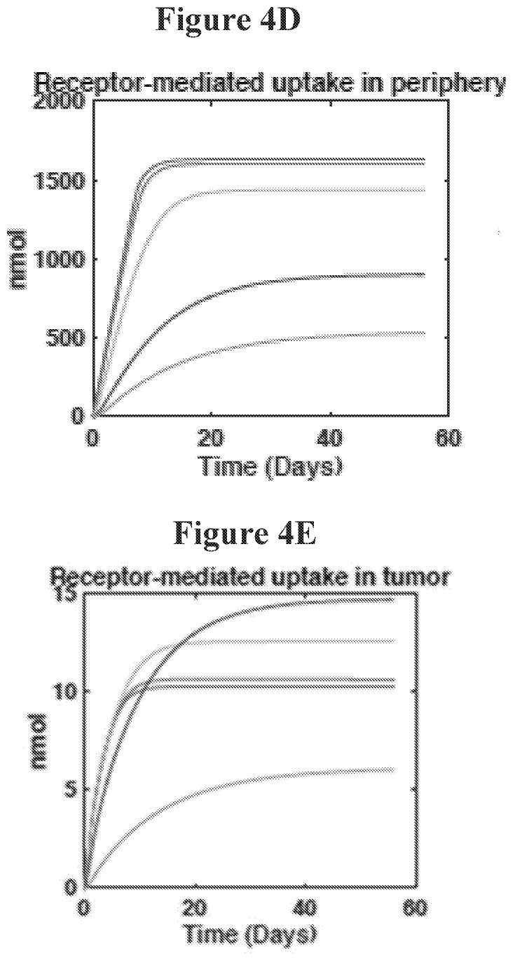

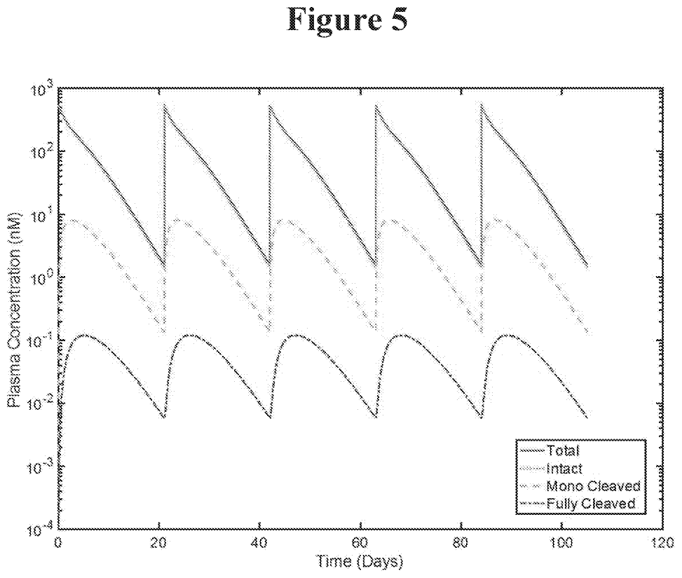

FIGS. 4A and 4B illustrates predictions of the QSP model of the present disclosure of the PK profiles in human plasma and periphery following a single 4.5 mg/kg dose of parental anti-CD166 mAb (0,0) and anti-CD166 activatable antibodies of increasing masking binding inhibition rations (Kₘₐₛₖ). In both plasma and peripheral compartments, parental mAb exposure is reduced relative to total activatable antibody, with evidence of a monotonic decrease in the terminal elimination rate with increasing Kₘₐₛₖ This observation is consistent with FIG. 4D, where model-predicted uptake in the periphery trends lower overall with increasing Kₘₐₛₖ, suggesting decreased contribution of TMDD in the periphery to the overall CD166 activatable antibody clearance with increasing Kₘₐₛₖ. The tumor compartment (FIG. 4C) follows trends of increasing exposure and decreasing terminal elimination rate with increasing Kₘₐₛₖ, respectively. FIG. 4D suggests the relationship of increasing Kₘₐₛₖ on receptor-mediated uptake in the tumor is not monotonic, and instead may pass through an optimum. Following multiple dose administration of 3 mg/kg of CD166 activatable antibodies, from FIG. 5 the QSP model of the present disclosure suggests that the intact CD166 activatable antibodies comprises the majority of the total circulating species in the plasma.

The QSP model of the present disclosure of the human PK of anti-CD166 activatable antibody demonstrates an enhanced therapeutic window offered by the activatable antibody relative to the parental mAb, as well as a platform to increase this window. For example, at a given dose level in the monkey, the exemplary observed data and model predictions showed a reduced exposure of circulating levels of parental anti-CD166 antibody relative to the anti-CD166 activatable antibody, and further showed a trend of increasing anti-CD166 activatable antibody exposure with increasing Kₘₐₛₖ (i.e. MM masking efficiency). Exemplary results in monkey further suggest that molecules sharing elements such as a given CM or MM could be described by a common estimate of k_{cleave} and Kₘₐₛₖ, respectively. Moreover, the QSP model of the present disclosure predicted in these examples that circulating activatable antibodies escape binding to targets in the peripheral compartment, thereby reducing the overall clearance rate.

The QSP model of the present disclosure applied in these examples to a human cancer model to human cancer patients likewise indicated a decreased systemic clearance of activatable antibodies relative to parental mAb, with correspondingly increased exposure of the activatable antibodies in the periphery and tumor. As observed in monkey, model-predicted changes in humans showed that activatable antibody exposure in plasma correlate with Kₘₐₛₖ in the QSP model of human cancer patients. Thus, enhanced circulating levels of activatable antibodies with increasing Kₘₐₛₖ, which further predicts that activatable antibody levels in the periphery and tumor (which equilibrate with the circulating levels) likewise increase with Kₘₐₛₖ.

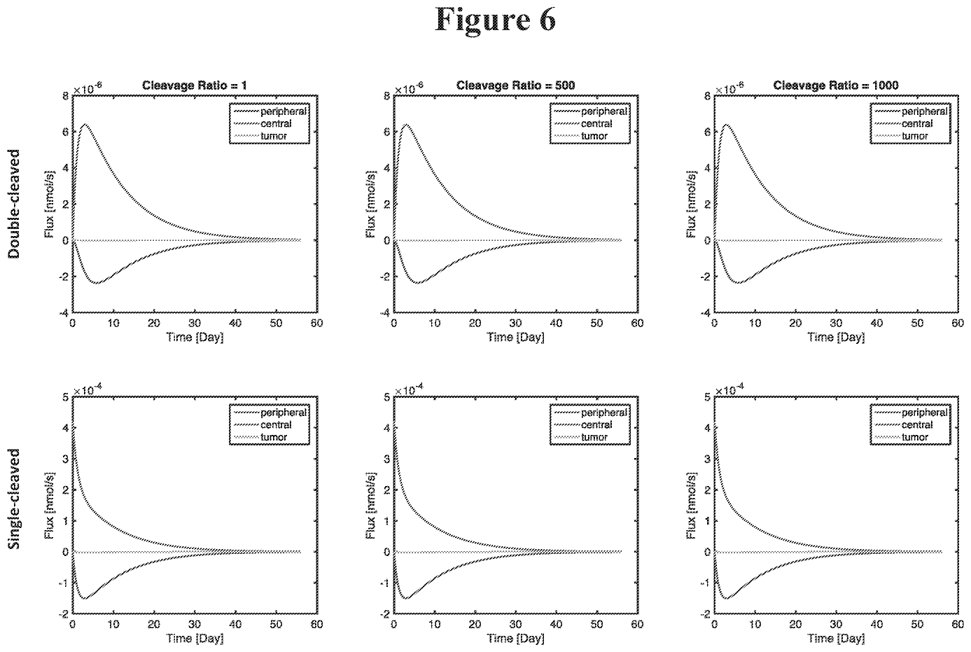

The parameter k_{cleave} in the QSP model of the present disclosure captures both CM substrate stability and protease activity. Under the exemplary scenario of a multiple higher-fold increased k_{cleave} in the tumor relative to periphery, as shown in FIG. 6, the exemplary QSP modeling predicted no net positive flux of cleaved species from tumor (i.e. tumor leakage of cleaved activated antibody) over a range of assumed fold increased k_{cleave} in the tumor relative to periphery.

### Example 3: Pharmacokinetics (PK) of Anti-PD-L1 Activatable Antibodies (AA) in Humans

In this clinical study, the observed levels of activatable antibody in plasma were determined following administration of an anti-PD-L1 activatable antibody (HC of SEQ ID NO: 224 and LC of SEQ ID NO: 225) to a subject at various dosage regimens.

In these exemplary studies, both intact and total (i.e., intact plus activated) activatable antibodies (AA) levels were determined in plasma samples using a validated high performance liquid chromatography tandem mass spectrometry (HPLC MS/MS) method with a lower limit of quantification for each analyte of 0.657 nM. Magnetic beads coated with protein A were used to enrich for immunoglobulin (including intact and activated activatable antibodies) in K₂EDTA plasma samples. The bound proteins were digested with trypsin, and two peptide fragments unique to the activatable antibody were monitored: one peptide from the anti-PD-L1 activatable antibody heavy chain that is present in both the intact and activated forms of the activatable antibody (for quantitation of total activatable antibody) and one peptide from the prodomain that is only present in the intact form of anti-PD-L1 activatable antibody (for quantitation of intact activatable antibody). Following the immunocapture and digestion steps, the final extract is analyzed via HPLC with MS/MS detection using positive ion electrospray.

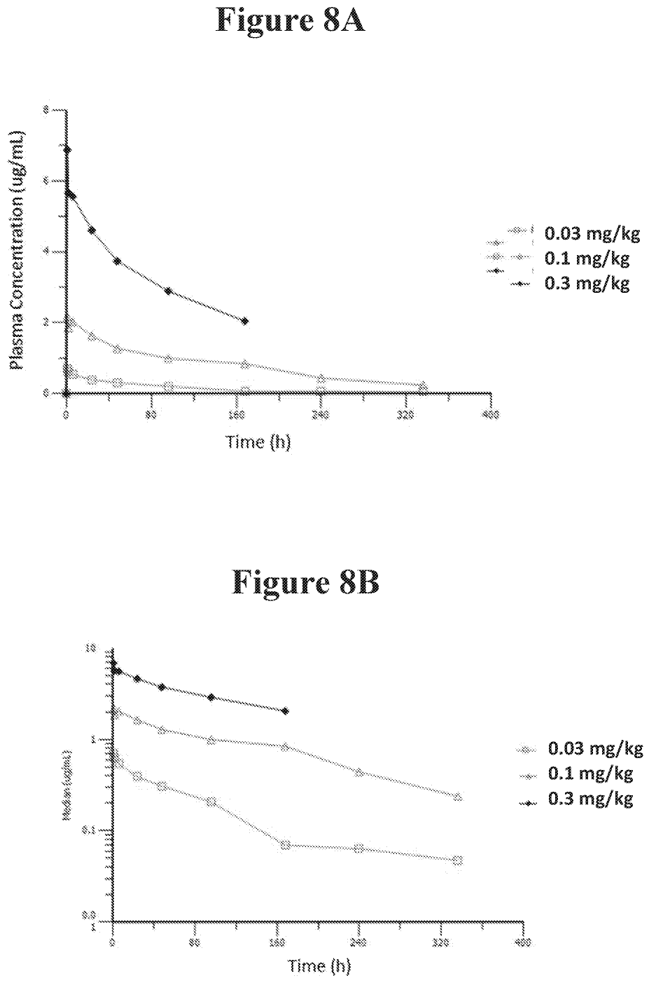

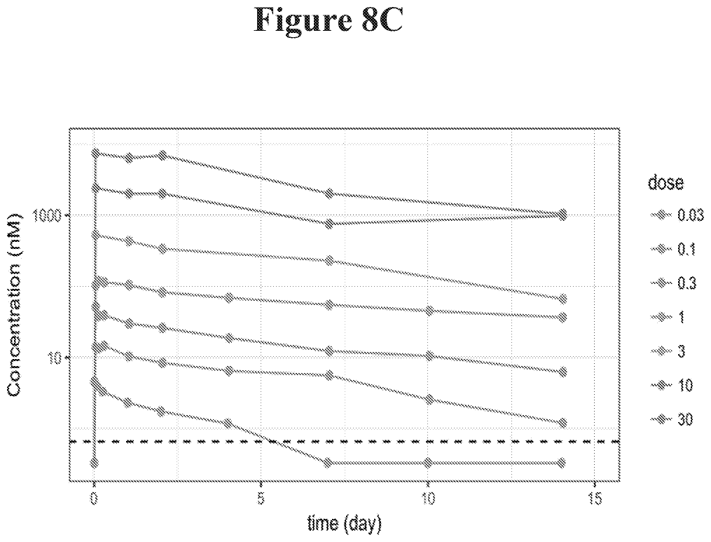

As shown in FIGS. 8A (linear) and 8B (semi-log), three different dosages of the activatable antibody (0.03 mg/kg, 0.1 mg/kg, and 0.3 mg/kg) were administered to subjects in a q2w regimen and the amount of intact activatable antibody were determined using the above-described method at the indicated time points. The median and range of AUCₜₐᵤ and Cₘₐₓ for the measured intact activatable antibodies in plasma for each dosage are summarized below in Table 3.

TABLE 3
PK Data for Anti-PD-L1 Activatable Antibody

| Dose | | AUC_{inf} | AUCₜₐᵤ | Cₘₐₓ | Cₘᵢₙ | CL | Vss |
|---|---|---|---|---|---|---|---|
| (mg/kg) | Analyte | (day*μg/mL) | (day*μg/mL) | (μg/mL) | (μg/mL) | (L/d) | (L) |
| 0.03 | Intact AA | 1.9 | 1.48 | 0.692 | — | 1.07 | 4.06 |
| | | [1.8-2] | [1.41-1.54] | [0.665-0.718] | | [0.851-1.28] | [3.34-4.79] |
| 0.03 | Total AA | 3.28 | 2.19 | 0.699 | 0.0948 | — | — |
| | | [2.45-4.12] | [1.6-2.77] | [0.617-0.781] | [0.0948-0.0948] | | |
| 0.1 | Intact AA | 12.4 | 10.8 | 2.5 | 0.315 | 0.562 | 3.51 |
| | | [8.6-16.2] | [7.56-14] | [2.31-2.68] | [0.315-0.315] | [0.554-0.57] | [3.16-3.85] |
| 0.1 | Total AA | 12.9 | 11.1 | 2.5 | 0.2375 | — | — |
| | | [9.24-16.6] | [8.36-13.9] | [2.4-2.6] | [0.136-0.339] | | |
| 0.3 | Intact AA | — | 33.7 | 7.2 | — | — | — |
| | | | [26.8-40.7] | [6.03-8.37] | | | |
| 0.3 | Total AA | — | 32.9 | 7.01 | — | — | — |
| | | | [26.3-39.5] | [5.59-8.43] | | | |

Values in shown in Table 3 are medians with their corresponding ranges shown in brackets ([ ]). AUC_{inf} is area under curve evaluated until infinity. AUCₜₐᵤ is area under curve evaluated until the end of the dosing interval. Cₘₐₓ is maximum plasma concentration. Cₘᵢₙ is minimum plasma concentration evaluated at tau. CL is clearance rate. Vss is volume of distribution.

The Cₘᵢₙ of the intact AA at the 0.03 mg/kg dosing regimen was below the level of quantitation. The other values not shown in Table 3 were not available at the time.

These exemplary results demonstrate that AUC_{inf}, AUCₜₐᵤ, and Cₘₐₓ were generally dose-proportional. The levels of intact activatable antibody were generally consistent with the levels of atezoluzimab (anti-PD-L1 monoclonal antibody) model predictions (see, e.g., Herbst, R. S., et al., “Predictive correlates of response to the anti-PD-L1 antibody NIPDL3280A in cancer patients.” Nature Vol. 515(7528), pp. 563-567 (2014).)

FIG. 8C shows the PK of anti-PD-L1 activatable antibody following its administration to subjects at the different indicated dosages (in mg/kg) that were administered to subjects in a q2w regimen. The median amount of intact activatable antibody for each dosage at the indicated time points was determined using the above-described method. The dotted horizontal line represents the lower limit of quantitation for the assay that was used. As shown in FIG. 8C, the median measured amount of plasma levels of intact activatable antibody correlated with the initial dosage, such that the highest median levels of intact AA in plasma resulting from administration of the highest dosage (30 mg/kg) are plotted as the uppermost line, and with each subsequent lower line representing the measured amount of intact AA resulting from each next lower dosage. These PK results are consistent with modeling of the activatable antibody at the same dosages using models of the present disclosure. In comparison, corresponding modeling of the parental, unmasked antibody (anti-PD-L1) show evidence of target-mediated drug disposition (TMIDD).

In another example, the levels of intact and total activatable antibody in cynomolgus monkey plasma were determined following administration of an anti-PD-L1 activatable antibody (HC of SEQ ID NO: 224 and LC of SEQ ID NO: 225) to the cynomolgus monkey following a single administration of 20 mg/kg, 60 mg/kg, or 200 mg/kg of the activatable antibody. These plasma levels were measured using standard protocols and using the methods described herein. Corresponding cynomolgus QSP models of the present disclosure were used to model the amount of circulating intact and total activatable antibody at the same administered dosages. The QSP models and observed results for both intact and total amounts of circulating activatable anti-PD-L1 antibody showed a high level of concordance over 7 days following the single administration.

### Example 4: QSP Modeling of Anti-PD-L1 Activatable Antibodies (AA) in Humans

The QSP model of the present disclosure was used to model an exemplary PK of an anti-PD-L1 activatable antibody (anti-PD-L1 AA) in a subject to determine the plasma concentration of the activatable antibody over time following administration of various dosing regimens to the subject.

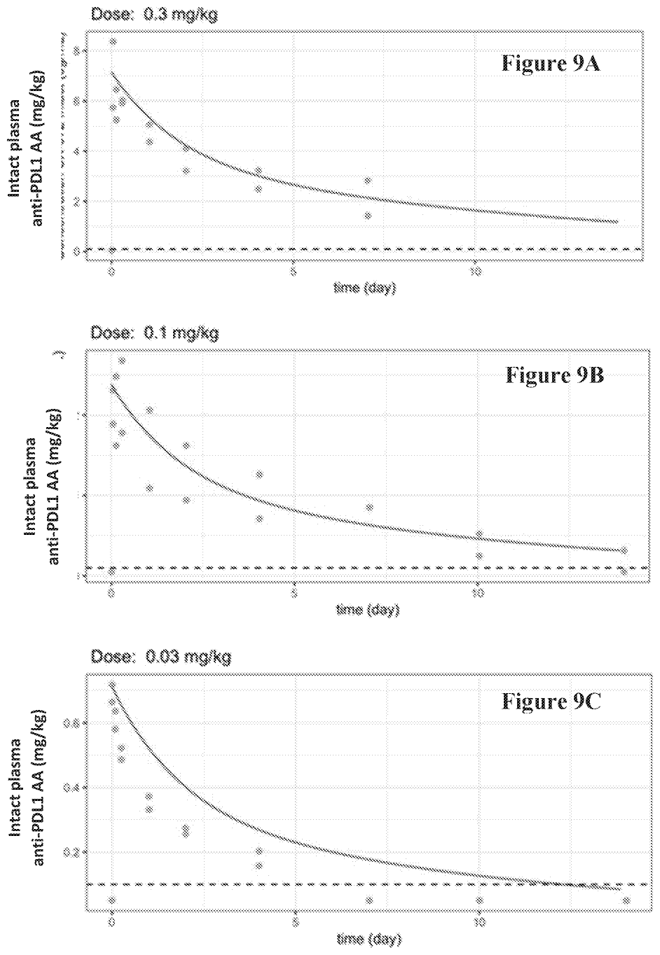

In this exemplary QSP model, the parameters were based on known or postulated mechanisms of activatable antibodies without reliance of observed human PK data of the corresponding activatable antibody. As shown in FIGS. 9A-9C, the observed PK of plasma levels of intact anti-PD-L1 activatable antibody (HC of SEQ ID NO: 224 and LC of SEQ ID NO: 225), which are indicated by the plotted dots in each graph, showed a high degree of concordance with PK as determined by the QSP model of the present disclosure, which are indicated by the curved lines in each graph. The QSP model of the present disclosure also showed that levels of intact and total (i.e., intact and activated) activatable antibody would be similar.

These examples of the QSP model of the present disclosure demonstrate, for example, that the observed PK of activatable antibodies are consistent with our mechanistic understanding of the behavior of activatable antibodies as shown by the QSP model.

### Example 5: QSP Modeling of the Dosage of Anti-PD-L1 Activatable Antibodies (AA) in Humans

The QSP model of the present disclosure was used to model the relationship between an administered dosage of an anti-PD-L1 activatable antibody (HC of SEQ ID NO: 224 and LC of SEQ ID NO: 225) with various periphery/tumor cleavage ratios in order to obtain a desired tumor receptor (or target) occupancy.

The QSP model in this example was based on fitting observed PK data of anti-PD-L1 activatable antibody with data of Herbst et al. (2014), as cited above, which showed modeling of the PK of a monoclonal anti-PD-L1 antibody. This fitting was used to estimate the elimination rate of the activated activatable antibody, the peripheral/plasma compartment distribution parameters, and the PD-L1 target expression levels, which were presumptively the same as that of anti-PD-L1 monoclonal antibody used in the Herbst model. The remaining QSP model parameters were based on literature references or derived from non-clinical data.

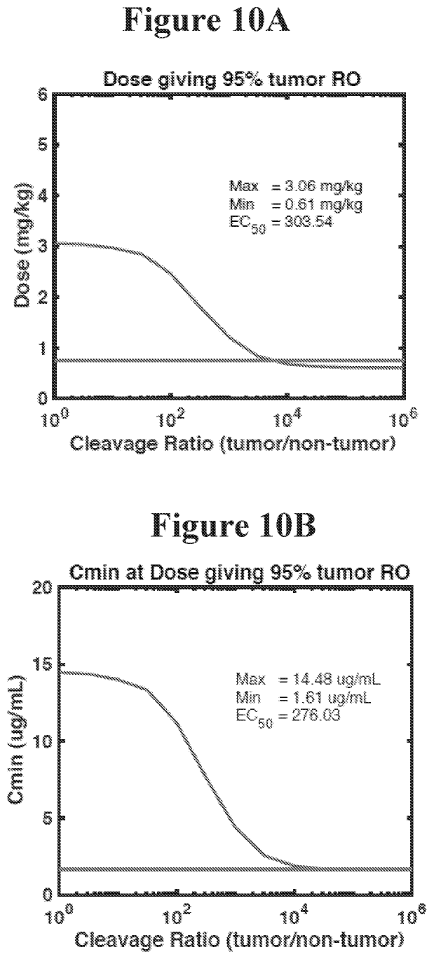

As shown in FIG. 10A, an exemplary QSP model of the present disclosure was used to determine the appropriate administered dose of activatable antibody (mg/kg) over a range of cleavage ratios to obtain a 95% receptor occupancy (RO) in the tumor. The cleavage ratio indicates the relative amount of cleavage of the activatable antibody in the tumor compartment as compared to the peripheral compartment. As shown in the exemplary modeled results of FIG. 10A, the amount of the required administered dosage of the activatable antibody decreased with increasing tumor/periphery cleavage ratio as shown by the curved line. The horizontal line indicates a similar model of a monoclonal antibody, which would be unaffected by cleavage ratios due to its lack of a prodomain. Similarly, as shown in FIG. 10B, an exemplary QSP model of the present disclosure was used to determine the Cₘᵢₙ of intact activatable antibody (mg/kg) over a range of cleavage ratios to obtain a 95% receptor occupancy (RO) in the tumor. The horizontal line indicates a similar model of a monoclonal antibody, which would be unaffected by cleavage rations due to its lack of a prodomain.

The results of these models indicate that, for example, the targeted Cₘᵢₙ, would range from 2 to 15 μg/mL. These models can be used, for example, to support dose selection in a clinical trial and used to interpret PK data from a clinical program.

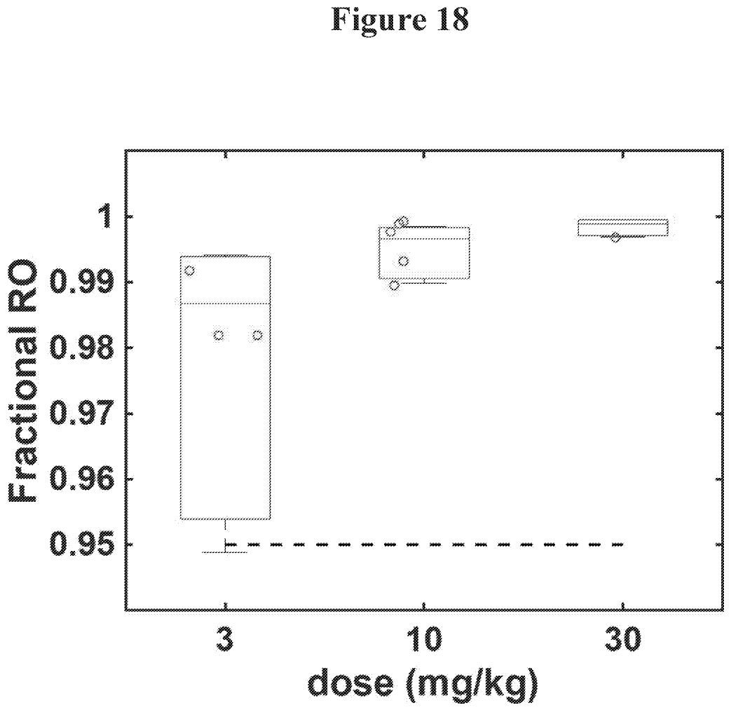

In another example, the QSP model of the present disclosure was used to predict the receptor occupancy (RO) in a tumor of an anti-PD-L1 activatable antibody (HC of SEQ ID NO: 224 and LC of SEQ ID NO: 225). Referring to FIG. 18, the QSP model of the present disclosure was used to model the RO following administration to cancer patients of 3 mg/kg, 10 mg/kg, or 30 mg/kg of the anti-PD-L1 activatable antibody. The box in the plot of FIG. 18 for a given dosage corresponds to the range of RO predicted by the QSP model of the present disclosure, based upon assumed relative rates of activatable antibody activation in the periphery to the tumor (relative rate spanning 1 to 1×10⁶). These predicted ROs corresponded to calculated estimated ROs based on patient tumor biopsies, which are indicated for each given dosage by points. These exemplary results demonstrate that the QSP model of the present disclosure can be used to predict RO for an activatable antibody

### Example 6: QSP Modeling of the PK of Isotope-Labeled Anti-PD-L1 Activatable Antibodies (AA) in Humans

The QSP model of the present disclosure was used to model the PK of an isotopically-labeled anti-PD-L1 activatable antibody in a subject to determine the plasma concentration of the activatable antibody over time following its administration in various dosing regimens to the subject.

In previous models of ⁸⁹Zr-labeled anti-PD-L1 monoclonal antibody atezoluzimab, low dosages of the monoclonal antibody were shown to have high clearance from the serum. For example, dosages of less than 1 mg of atezoluzimab were cleared from the serum in 7-21 days after administration. Accordingly, additional “cold” (i.e. unlabeled) monoclonal antibody in the amount of 10 mg was administered concurrently with 1 mg labeled monoclonal antibody to mitigate the rapid clearance of the labeled monoclonal antibody.

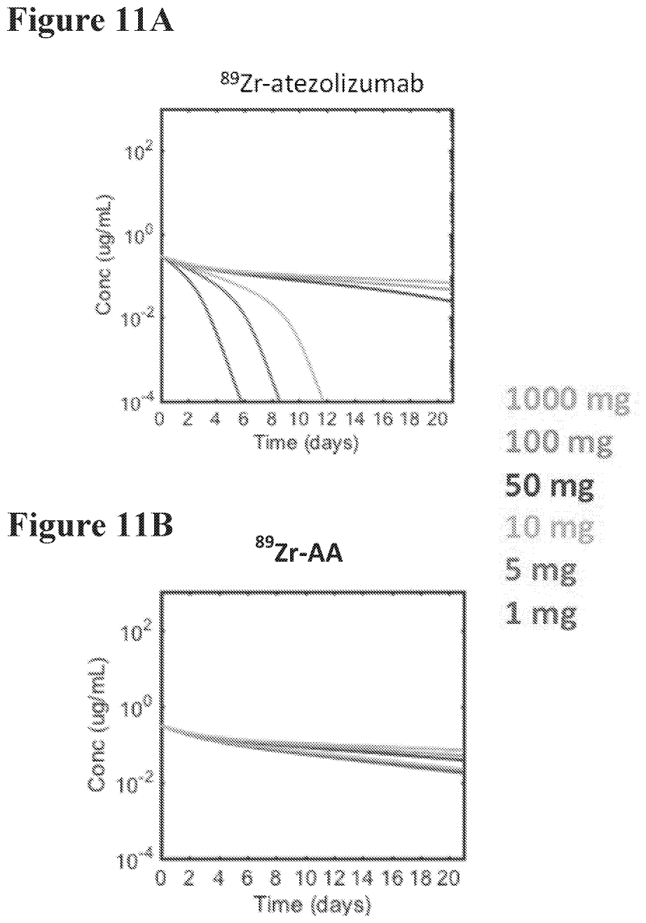

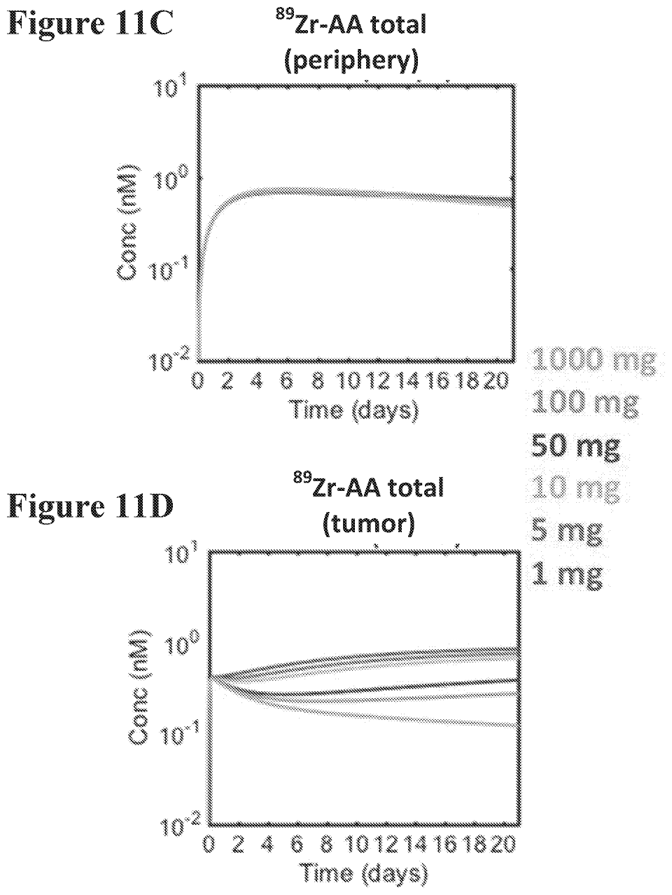

Using the QSP model of the present disclosure, the PK of serum levels of anti-PD-L1 activatable antibody (HC of SEQ ID NO: 224, LC of SEQ ID NO: 225) were modeled at varying dosages, as shown in FIGS. 11A and 11B. In FIG. 11A, a QSP model was used to determine the levels of circulating ⁸⁹Zr-labeled monoclonal antibody atezoluzimab following administration of 1 mg of the labeled monoclonal antibody along with the indicated amount (ranging from 1 to 1000 mg) of the unlabeled monoclonal antibody. The exemplary model depicted in FIG. 11A demonstrated that the levels of circulating ⁸⁹Zr-labeled monoclonal antibody increased with increasing amounts of co-administered unlabeled antibody. In FIG. 11B, a QSP model was used to determine the levels of circulating ⁸⁹Zr-labeled anti-PD-L1 activatable antibody following administration of 1 mg of the labeled activatable antibody along with the indicated amount (ranging from 1 to 1000 mg) of the unlabeled activatable antibody. The exemplary model depicted in FIG. 11B demonstrated that the levels of circulating ⁸⁹Zr-labeled activatable antibody did not markedly increase with increasing amounts of co-administered unlabeled antibody. These results also show that, for example, lower amounts of co-administered unlabeled activatable antibody was required to maintain circulating levels as compared to the monoclonal antibody.

As shown in FIGS. 11C and 11D, the exemplary QSP modeling results demonstrated that, for example, after co-administering ⁸⁹Zr-labeled anti-PD-L1 activatable antibody with 1-1000 mg of unlabeled activatable antibody as indicated, the QSP model indicated that levels of the label in the tumor compartment (FIG. 11D) decreased with increasing amounts of co-administered unlabeled activatable antibody, while the amount of label indicated by the QSP model in the peripheral compartment (FIG. 11C) remained essentially the same.

### Example 7: QSP Modeling of the Cleavage of Anti-PD-L1 Activatable Antibodies (AA)

The QSP model of the present disclosure was used to model the level of activated anti-PD-L1 activatable antibody in a tumor compartment of a subject.

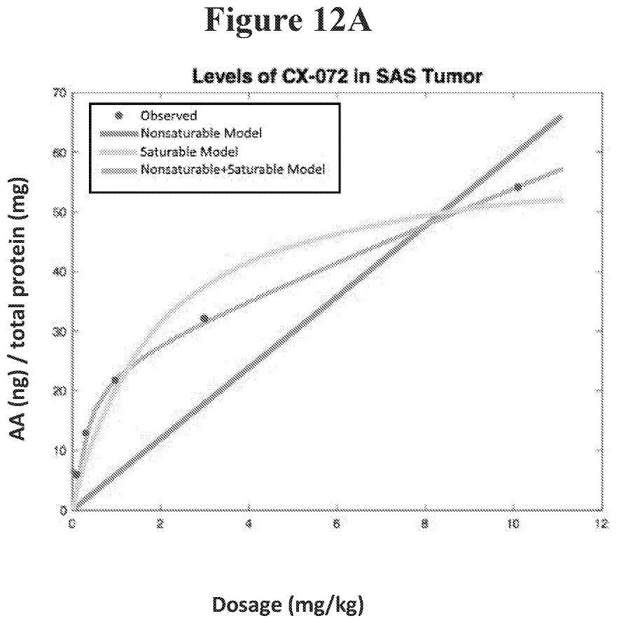

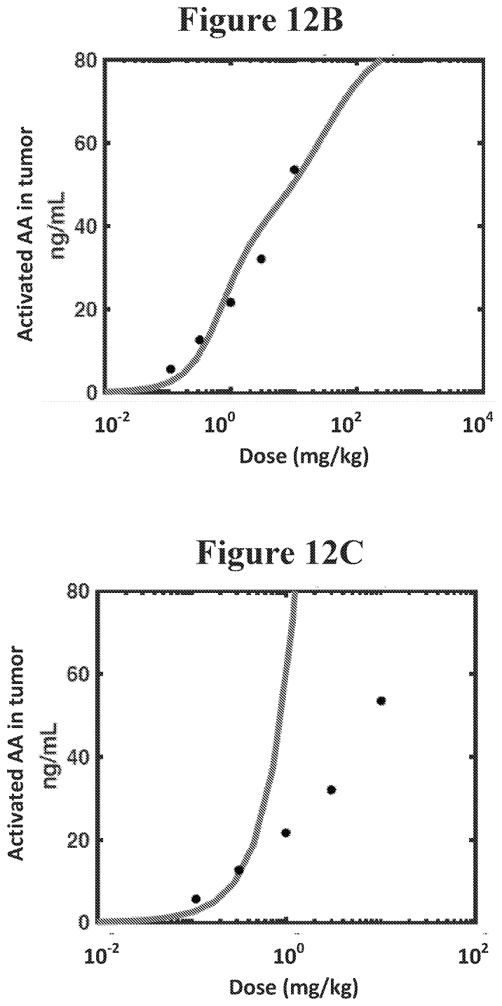

As shown in FIG. 12A, the amount of activated anti-PD-L1 activatable antibody formed in mouse xenograft tumors was observed to be relatively higher at lower dosages of the activatable antibody, indicative of a non-saturated phase and a saturated phase for kinetics.

Using the QSP model of the present disclosure, in an exemplary study the cleavage of the activatable antibody was modeled to assess different parameters of the protease. In the first model (FIG. 12B), the protease was modeled with a low Km (˜nM range), and the model demonstrated that in this scenario with increasing dosages of activatable antibody, the protease becomes saturated by the substrate (i.e., the AA), resulting in increasing amounts of intact activatable antibody with increasing dosages. As a result, the intact activatable antibody at these higher dosages begins to compete with the activated activatable antibody for binding to the target and hence also competes with the clearance mechanism of activatable antibody from the tumor. Thus, the model demonstrates in this scenario the rate of clearance of the activated activatable antibody from the tumor compartment is reduced, thus producing an apparent continued increase in activated activatable antibody in the tumor compartment. As shown in FIG. 12B, this model appears consistent with the observed amounts of activated activatable antibody in the tumor compartment.

In the second model (FIG. 12C), the protease was modeled with a high Km (˜μM range, typical of MMP and TACE enzymes), and the model demonstrated that in this scenario, the amount of intact activatable antibody is in the linear kinetic range for dosages of 1-20 mg/kg. As shown in FIG. 12C, this model appears inconsistent with the observed amounts of activated activatable antibody in the tumor compartment.

### Example 8: QSP Modeling of the Dosage of Anti-PD-1 Activatable Antibodies (AA) in Humans

In this example, the QSP model of the present disclosure was used to estimate the biologically effective dose (BED) of an anti-PD-1 activatable antibody by modeling the relationship between an administered dosage of the anti-PD-1 activatable antibody (SEQ ID NOs: 226 (heavy chain) and 227 (light chain)) with various mechanisms of receptor binding, cleavage, elimination, tissue and tumor biodistribution, and receptor and receptor-drug complex endocytosis in order to obtain a desired tumor receptor (or target) occupancy.

In this example, the estimated BED for the anti-PD-1 activatable antibody was based on models of the relationship between the administered dosage of the anti-PD-1 monoclonal antibody pembrolizumab and its target receptor occupancy. For pembrolizumab, population PK/PD modeling suggested that 2 mg/kg and 10 mg/kg of pembrolizumab administered every three weeks was at or near the plateau of response in melanoma patients. (See, e.g., Chatterj ee M. S., et al.; “Population Pharmacokinetic/Pharmacodynamic Modeling of Tumor Size Dynamics in Pembrolizumab-Treated Advanced Melanoma.” CPT: Pharmacometrics & Systems Pharmacology (2017) 6(1), pp. 29-39.) Simulations further suggested that a 50% reduction in exposure relative to the typical exposure following 2 mg/kg pembrolizumab (i.e. the typical exposure associated with pembrolizumab is 1 mg/kg administered every third week (q3w)) would not be clinically meaningful. Available analyses also suggested a flat exposure-response relationship for patients receiving 1 mg/kg to 10 mg/kg pembrolizumab in melanoma, and this interval includes the marketed 2 mg/kg and 200 mg fixed dose levels.

In this example, the BED for the anti-PD-1 activatable antibody was based on a QSP model-estimated tumor receptor occupancy (RO) of the activatable antibody that corresponded to the RO of pembrolizumab following a 1 mg/kg dose level. A BED of 80 mg (range 80 to 240 mg) is the predicted dose of anti-PD-1 activatable antibody required to generate the QSP model-estimated 99.5% tumor RO, which is the same RO that is modeled to follow administration of pembrolizumab at 1 mg/kg.

As discussed herein, a QSP model of the present disclosure was developed to capture known mechanisms of activatable antibody receptor binding, cleavage, elimination, tissue and tumor bio-distribution, and receptor and receptor-drug complex endocytosis. This QSP model of the present disclosure used in this example included four (4) different parameterizations. The first and second parameterizations captured cynomolgus monkey PK data following anti-PD-1 activatable antibody and a corresponding anti-PD-1 monoclonal antibody (SEQ ID NOs: 226 (heavy chain) and 228 (light chain)). The anti-PD-1 monoclonal antibody shares the same heavy and light chain sequence as the anti-PD-1 activatable antibody, but the former lacks the prodomain. The third and fourth parameterizations correspond to human models following administration of pembrolizumab and the anti-PD-1 activatable antibody, respectively, and are hereafter referred to as the “human pembrolizumab QSP model” and “human anti-PD-1 activatable antibody QSP model,” respectively. Parameters for the binding of the anti-PD-1 monoclonal antibody and anti-PD-1 activatable antibody were derived from in vitro binding data.

Both monkey QSP models of the present disclosure for anti-PD-1 monoclonal antibody and anti-PD-1 activatable antibody were calibrated to monkey PK data following administration of the respective molecules to cynomolgus monkeys. The human pembrolizumab QSP model of the present disclosure was used to estimate the concentration of PD-1 in humans, and the human anti-PD-1 activatable antibody QSP model of the present disclosure was parametrized based upon the integration of outputs from the previous three steps. The human anti-PD-1 activatable antibody QSP model of the present disclosure was finally used to estimate the likely BED for anti-PD-1 activatable antibody.

TABLE 4
Exemplary Parameters in QSP Model for Anti-PD-1
Antibody (mAb) and Activatable Antibody (ActAb)

| | | | Human | | | |
|---|---|---|---|---|---|---|
| | Cyno | Cyno | PD-1 | Human | | |
| Parameter | PD-1 | PD-1 | Pembro | PD-1 | | |
| (Unit) | mAb | ActAb | mAb | ActAb | Notes | Ref. |
| Mol. Wt. (kDa) | 144.75 | 153.72 | 146 | 153.72 | | |
| Body Wt. (kg) | 3 | 3 | 77 | 77 | Based on typical or avg. values | (4, 5) |
| Central vol. Vc | 0.09 | 0.09 | 4.06 | 3.3 | Cyno PD-1 ActAb based on fit | (6) |
| (L) | | | | | to Cmax | |
| | | | | | Human PD-1 ActAb scaled | |
| | | | | | value for 77 kg human | |
| | | | | | Pembro mAb: ref. 6 | |
| Peripheral vol., | 0.09 | 0.09 | 3.48 | 3.3 | Cyno & human PD-1 ActAb | (6) |
| Vp (L) | | | | | set equal to central | |
| | | | | | compartment | |
| | | | | | Pembro mAb: ref. 6 | |
| Tumor vol., Vt | N/A | N/A | 0.01 | 0.01 | In the range of breast tumor | |
| (L) | | | | | ER/PR+ | |
| K_{cleave} (sec⁻¹) | N/A | 1.84e−7 | N/A | 8.17e−8 | Cyno PD-1 ActAb fit to | |
| | | | | | experimental data. | |
| | | | | | Human PD-1 ActAb | |
| | | | | | allometrically scaled from cyno | |
| | | | | | value. | |
| Elimination | 8.89e−7 | 6.71e−7 | 7.32e−7 | 2.98e−7 | Cyno PD-1 ActAb fit to | (6) |
| rate constant, | | | | | experimental data. | |
| kₑₗ (sec⁻¹) | | | | | Human PD-1 ActAb | |
| | | | | | allometrically scaled from cyno | |
| | | | | | value. | |
| | | | | | Pembro mAb: ref. 6 | |
| K₁₂ (sec⁻¹) | 7.5e−6 | 1.15e−5 | 2.64e−6 | 5.13e−6 | Cyno PD-1 ActAb fit to | (6) |
| | | | | | experimental data. | |
| | | | | | Human PD-1 ActAb | |
| | | | | | allometrically scaled from cyno | |
| | | | | | value. | |
| | | | | | Pembro mAb: ref. 6 | |
| K₂₁ (sec⁻¹) | 5.52e−6 | 8.33e−6 | 2.27e−6 | 3.70e−6 | Cyno PD-1 ActAb fit to | (6) |
| | | | | | experimental data. | |
| | | | | | Human PD-1 ActAb | |
| | | | | | allometrically scaled from cyno | |
| | | | | | value. | |
| | | | | | Pembro mAb: ref. 6 | |
| K₁₃ (sec⁻¹) | N/A | N/A | 5.93e−10 | 1.12e−9 | Calculated based on partition | |
| | | | | | coefficient | |
| K₃₁ (sec⁻¹) | N/A | N/A | 4.91e−6 | 8.83e−6 | Calculated based on partition | |
| | | | | | coefficient | |
| Tumor/plasma | N/A | N/A | 0.21 | 0.21 | Based on literature value for | (7) |
| partition (sec⁻¹) | | | | | atezolizumab | |
| Kₒₚₑₙ (sec⁻¹) | N/A | 0.0116 | N/A | 0.0116 | Assumed | |
| K_{close} (sec⁻¹) | N/A | 0.9663 | N/A | 0.9663 | Estimated as Kmask*kopen | |
| Kₒₙ (nM⁻¹ sec⁻¹) | 1e−3 | 1e−3 | 1e−3 | 1e−3 | Typical Kon value from | (8) |
| | | | | | literature | |
| K_{off} (sec⁻¹) | 7.73e−5 | 7.73e−5 | 5.8e−5 | 7.73e−5 | Computed from Kd and Kon | |
| Central | 8.66e−8 | 8.66e−8 | 2.68e−6 | 2.54e−6 | Fit to human, low dose | (9) |
| K_{syn}_{_{—}}PD1 | | | | | pembrolizumab TMDD data | |
| (nmol/sec) | | | | | | |
| Periph. | 8.66e−8 | 8.66e−8 | 3.13e−6 | 2.54e−6 | Fit to human, low dose | (9) |
| K_{syn}_{_{—}}PD1 | | | | | pembrolizumab TMDD data | |
| (nmol/sec) | | | | | | |
| Tumor | N/A | N/A | 1.54e−9 | 1.54e−9 | Assumed same as | |
| K_{syn}_{_{—}}PD1 | | | | | Central/Periph expression | |
| (nmol/sec) | | | | | | |
| K_{endo}_{_{—}}PD1 | 9.63e−5 | 9.63e−5 | 9.63e−5 | 9.63e−5 | Assumed 2 hr turnover rate | |
| (sec⁻¹) | | | | | | |
| K_{D} (pM) of | | | 29 | | Fixed based on its typical value | (4) |
| pembrolizumab | | | | | in literature | |
| K_{D} (pM) of | 39 | | | | Estimated based on ELISA | |
| anti-PD-1 | | | | | assay | |
| monoclonal | | | | | | |
| antibody | | | | | | |

### References for Table 4:

(4) Stroh M, Winter H, Marchand M, Claret L, Eppler S, Ruppel J, et al. The Clinical Pharmacokinetics and Pharmacodynamics of Atezolizumab in Metastatic Urothelial Carcinoma. Clin Pharmacol Ther. 2016.
(5) Davies B, Morris T. Physiological parameters in laboratory animals and humans. Pharm Res. 1993; 10(7):1093-5.
(6) Ahamadi M, Freshwater T, Prohn M, Li C H, de Alwis D P, de Greef R, et al. Model-Based Characterization of the Pharmacokinetics of Pembrolizumab: A Humanized Anti-PD-1 Monoclonal Antibody in Advanced Solid Tumors. CPT: pharmacometrics & systems pharmacology. 2017; 6(1):49-57.
(7) Deng R, Iyer S, Theil F P, Mortensen D L, Fielder P J, Prabhu S. “Projecting human pharmacokinetics of therapeutic antibodies from nonclinical data: what have we learned?” MAbs. 2011; 3(1):61-6.
(8) Schlosshauer M, Baker D. Realistic protein-protein association rates from a simple diffusional model neglecting long-range interactions, free energy barriers, and landscape ruggedness. Protein Sci. 2004; 13(6):1660-9.
(9) Patnaik A, Kang S P, Rasco D, Papadopoulos K P, Elassaiss-Schaap J, Beeram M, et al. Phase I Study of Pembrolizumab (MK-3475; Anti-PD-1 Monoclonal Antibody) in Patients with Advanced Solid Tumors. Clinical cancer research: an official journal of the American Association for Cancer Research. 2015; 21(19): 4286-93.

In this exemplary QSP model of the present disclosure, the dissociation constant (K_{D}) for pembrolizumab of 29 pM was assumed from the literature. (Ref (4); Stroh et al.) The K_{D} of 39 pM for anti-PD-1 monoclonal antibody of 39 pM was estimated based on experimental ELISA assays. In this example, as neither kₒₙ₁ nor k_{off1} could not be separately determined by these assays kₒₙ₁ was fixed at 0.001 nM⁻¹ s⁻¹ which is a typical value for a monoclonal antibody receptor binding (Ref. (8); Schlosshauer, et al.). The off rate was then computed using k_{off1}=kₒₙ₁*K_{D}.

In this exemplary QSP model, it was assumed that the binding of fully activated anti-PD-1 activatable antibody would be similar to that for the corresponding anti-PD-1 monoclonal antibody. The Kₘₐₛₖ of 83.3 was then calculated as follows:

Kₘₐₛₖ=K_{app_PD-1 ActAb}/K_{app_PD-1 mAb}

Where K_{app_PD-1 mAb} and K_{app_PD-1 ActAb} of 0.320 nM and 26.7 nM, respectively, were the apparent affinity constants as obtained from a cell binding assay.

Calibration to Cynomolgus Monkey Data Exemplary Results

In this exemplary study, the monkey anti-PD-1 monoclonal antibody QSP model and the monkey anti-PD-1 activatable antibody QSP model were calibrated to monkey PK data following administration of anti-PD-1 monoclonal antibody and anti-PD-1 activatable antibody, respectively. Though all animals entered into this evaluation tested positive for anti-drug antibodies, only a subset of the animals exhibited decreased exposure in the elimination phase at Days 14 and after. Animals which exhibited decreased exposure in the elimination phase at Days 14 and after were identified by inspection and were excluded from the calibration. Anti-PD-1 monoclonal antibody at the 1 and 5 mg/kg dose levels were fit individually, as were anti-PD-1 activatable antibody data at the 5 mg/kg dose level. Parameters k₁₂, k₂₁, kₑₗ were estimated using the optimization package within KroneckerBio 0.5 using a log-weighted sum of squares objective function and a linear error function with constant and proportional coefficients of 0 and 0.1, respectively. The volume of the central compartment (Vc) was estimated from the equation Vc=Cmax/D, where Cmax is the observed maximum plasma concentration of anti-PD-1 monoclonal antibody and anti-PD-1 activatable antibody, respectively, in nM following the dose D of anti-PD-1 monoclonal antibody and anti-PD-1 activatable antibody, respectively, in nmol. Body weights were assumed to a value of 3 kg for each animal.

Table 4 provides the model parameters for calibration of the monkey anti-PD-1 monoclonal antibody QSP model and monkey anti-PD-1 activatable antibody QSP model to the observed cynomolgus monkey PK data following administration of anti-PD-1 monoclonal antibody and anti-PD-1 activatable antibody, respectively. The Vc estimate was 90 mL, and the peripheral volume of distribution (Vp) was assumed equal to Vc. For anti-PD-1 activatable antibody, k₁₂, k₂₁, kₑₗ were estimated to be 1.15e-5 sec⁻¹, 8.33e-6 sec⁻¹, and 6.71e-7 sec¹, respectively. For anti-PD-1 monoclonal antibody, the geometric average of the k₁₂, k₂₁, kₑₗ estimates obtained at the 1 mg/kg and 5 mg/kg CX-083 dose levels were 1.46e-5 sec⁻¹, 8.73e-6 sec⁻¹, and 8.63e-7 sec⁻¹, respectively.

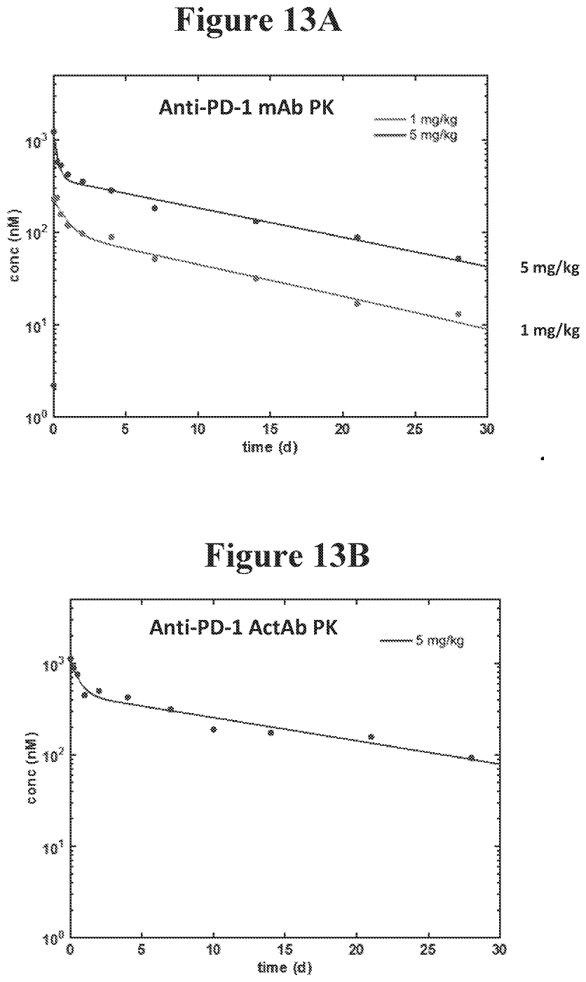

The monkey anti-PD-1 monoclonal antibody QSP model-(FIG. 13A) and monkey anti-PD-1 activatable antibody QSP model-predicted (FIG. 13B) PK data adequately captured the observed anti-PD-1 monoclonal antibody and anti-PD-1 activatable antibody data, respectively. In FIGS. 13A and 13B, the solid lines indicate model-estimated PK and the dots indicate observed PK for the indicated molecules and dosages in cynomolgus monkeys.

Available cynomolgus monkey from studies of the anti-PD-1 activatable antibody at the 100 mg/kg dose level suggested an approximate 20% decrease in the fraction of the activatable antibody that was circulating in plasma as the intact moiety over a 7-day period. The k_{cleave} was estimated using this observed decrease in the fraction intact of the anti-PD-1 activatable antibody using the monkey anti-PD-1 activatable antibody QSP model.

Other model parameters along with information pertaining to source of these parameters are summarized in Table 4.

Human Model Development Exemplary Results

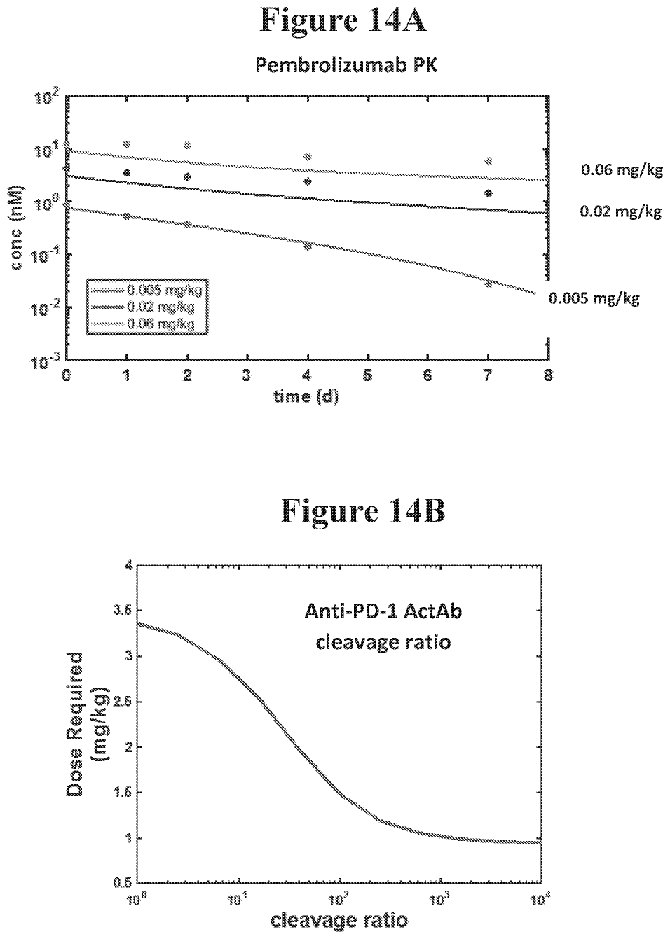

Table 4 provides the model parameters for estimation of PD-1 levels in human. The PD-1 Css was estimated to be 8.3 pM. As shown by the exemplary data in FIG. 14A, the human pembrolizumab QSP model adequately captured the low-dose pembrolizumab PK data, where the solid lines indicate model-estimated PK and the dots indicate observed PK for the indicated dosages.

Using the human pembrolizumab QSP model, the tumor RO for pembrolizumab 1 mg/kg would be approximately 99.5%.

From FIG. 14B, results from the human anti-PD-1 activatable antibody QSP model of the present disclosure suggest that the dose required for the anti-PD-1 activatable antibody to achieve 99.5% tumor RO would be approximately 1 mg/kg in the limit of high ratio (10000×) of k_{cleave} in the tumor relative to k_{cleave} the periphery (cleavage ratio). In the limit of low cleavage ratio (1×), results from the human CX-188 QSP model suggest that a dose of approximately 3 mg/kg would be required to achieve the same targeted tumor RO; when evaluated at the midpoint of these cleavage ratios (100×) the anti-PD-1 activatable antibody dose required is approximately 1 mg/kg. Assuming a 80 kg body weight, the fixed dose equivalent to the 1 to 3 mg/kg dose range is 80 to 240 mg. Thus, the 80 mg (ranging from 80 to 240 mg) dose is the predicted BED for the anti-PD-1 activatable antibody based on the QSP model of the present disclosure.

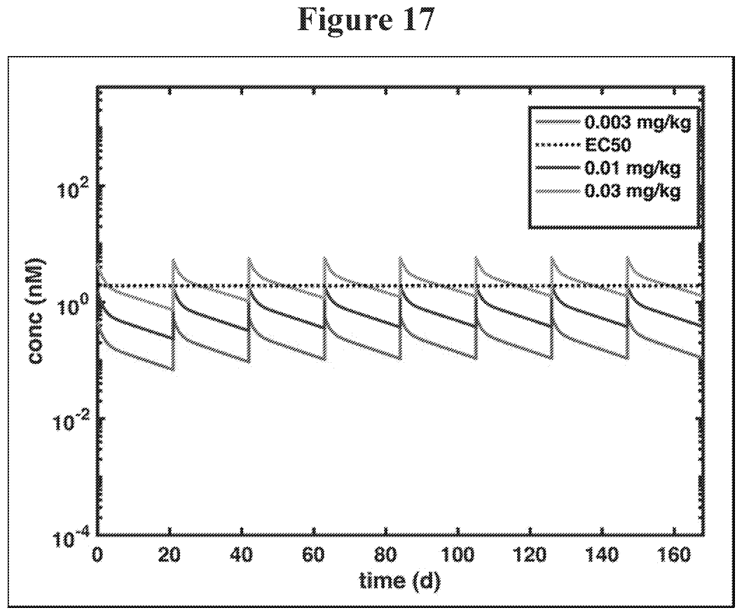

From FIG. 17, the results from an exemplary human QSP model of the present disclosure were used to estimate the dose required for the anti-PD-1 activatable antibody (HC of SEQ ID NO: 226 and light chain of SEQ ID NO: 227) to achieve the MABEL dose (i.e., the estimated dose that just exceeds the EC50 of 0.251 ug/mL from an in vitro cytomegalovirus (CMV) T cell recall assay, which is defined as MABEL). This in vitro CMV antigen recall assay in which activatable anti-PD-1 antibody enhanced T cell activation in primary human peripheral blood mononuclear cells (PBMCs) with an EC50 of 0.251 μg/mL. From inspection of the QSP-modeled results of dosages of 0.003 mg/kg, 0.01 mg/kg, and 0.03 mg/kg shown in FIG. 17, following administration of 0.01 mg/kg anti-PD-1 activatable antibody, Cₘₐₓ values would just surpass the EC50 of 0.251 ug/mL (1.6 nM) from an in vitro CMV antigen recall assay performed with primary human PBMCs. Following administration of 0.01 mg/kg anti-PD-1 activatable antibody, QSP model-simulations suggest that the activatable antibody would predominantly circulate as the intact species (96% ratio of model-predicted areas under the curve for intact to total anti-PD-1 activatable antibody following a multiple doses of 0.01 mg/kg anti-PD-1 activatable antibody (see Table 5A-5D). Accordingly, no additional correction was made for the possible contribution of activated activatable antibody.

TABLE 5A
Activatable Anti-PD-1 Antibody (ActAb) Q3Wx1

| Dose | Total ActAb | Intact ActAb | Total ActAb | Intact ActAb |
|---|---|---|---|---|
| (mg/kg) | Cₘₐₓ (nM) | Cₘₐₓ (nM) | AUC (nM*day) | AUC (nM*day) |
| 0.01 | 1.58 | 1.58 | 10.2 | 9.91 |
| 0.1 | 15.8 | 15.8 | 111 | 103 |
| 0.3 | 47.3 | 47.3 | 347 | 314 |
| 1 | 158 | 158 | 1180 | 1050 |
| 3 | 473 | 473 | 3550 | 3160 |
| 10 | 1580 | 1580 | 11900 | 10500 |

TABLE 5B
Activatable Anti-PD-1 Antibody (ActAb) Q3Wx5

| Dose | Total ActAb | Intact ActAb | Total ActAb | Intact ActAb |
|---|---|---|---|---|
| (mg/kg) | Cₘₐₓ (nM) | Cₘₐₓ (nM) | AUC (nM*day) | AUC (nM*day) |
| 0.01 | 1.98 | 1.96 | 15.8 | 15.2 |
| 0.1 | 22.2 | 20.5 | 213 | 172 |
| 0.3 | 69.9 | 61.8 | 712 | 522 |
| 1 | 237 | 206 | 2470 | 1740 |
| 3 | 716 | 619 | 7490 | 5230 |
| 10 | 2390 | 2060 | 25100 | 17500 |

TABLE 5C
Activatable Anti-PD-1 Antibody (ActAb) Q4Wx1

| Dose | Total ActAb | Intact ActAb | Total ActAb | Intact ActAb |
|---|---|---|---|---|
| (mg/kg) | Cmax (nM) | Cmax (nM) | AUC (nM*day) | AUC (nM*day) |
| 13.75 | 2170 | 2170 | 19700 | 16900 |

TABLE 5D
Activatable Anti-PD-1 Antibody (ActAb) Q4Wx5

| Dose | Total ActAb | Intact ActAb | Total ActAb | Intact ActAb |
|---|---|---|---|---|
| (mg/kg) | Cmax (nM) | Cmax (nM) | AUC (nM*day) | AUC (nM*day) |
| 13.75 | 2980 | 2610 | 35900 | 24300 |

Based on these exemplary results, and assuming an 80 kg body weight, the fixed-dose equivalent of the 0.01 mg/kg dose is 0.8 mg. Thus, these exemplary results show that the QSP model of the present disclosure can be used to estimate dosages to achieve particular efficacies.

### Example 9: QSP Modeling of the Dosage of Activatable T-Cell Bispecific Antibodies in Humans

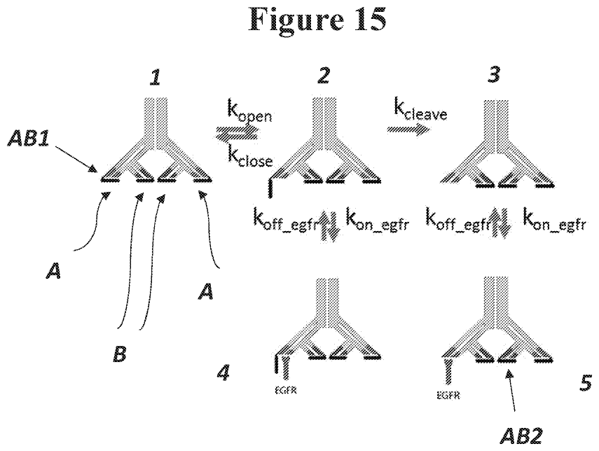

In this example, the QSP model of the present disclosure was used to estimate the biologically effective dose (BED) of activatable T-cell bispecific antibodies. As shown in FIG. 15, the QSP model of this embodiment of the present disclosure accounts for various species of activatable and activated T-cell bispecific antibodies and conversion pathways therebetween in a manner that is analogous to the various species of activatable mono-specific antibodies described herein. For example, as shown in FIG. 15, a schematic depiction of an exemplary activatable T-cell bispecific antibody is depicted, showing conversion between the various activatable and activated forms of the T-cell bispecific antibody. In this exemplary depiction of an activatable T-cell bispecific antibody, the activatable bispecific antibody (item no. 1) includes two target-specific binding elements (AB1 and AB2) each with a masking prodomain (A) and two T-cell specific binding elements each with a masking prodomain (B). This figure also schematically depicts different species of activated bispecific antibody, including an activated bispecific antibody with one conformationally unmasked prodomain (item no. 2), and an activated bispecific antibody with one prodomain removed, e.g., by cleavage, from the activatable bispecific antibody (item no. 3). The figure also depicts that the conformationally activated or cleavage activated bispecific antibody can reversibly bind to its target (item nos. 4 and 5, respectively). Other activated forms of the bispecific antibody are also envisioned but not shown here, such as where each prodomain can be reversibly and conformationally unmasked or irreversibly cleaved.

The exemplary conversion reactions and various species of activatable and activated bispecific antibodies can be present in various compartments (also in a manner that is analogous to the various compartments and transfers therebetween in relation to mono-specific activatable antibodies as described herein), which are used in the QSP model of the present disclosure. As explained herein, compartments represent portions of a subject's body, and typically include at least one target compartment and at least one non-target compartment. For example, the non-target compartment may represent, at least, a plasma compartment of the subject. In another example, non-target compartments can include a central compartment, which may be a plasma compartment, and a “peripheral” compartment that represents one or more non-target organs or tissues in the subject. A peripheral compartment may or may not be physiological; e.g., it may contain multiple non-target organs/tissues, or even a subset of one organ or tissue. In each compartment, the various species of activatable and activated (partially and fully) of the T-cell bispecific antibody can be present.

Compartments are generally chosen because they are relevant to determining a time-dependent concentration or amount (mass or density) of activatable bispecific antibody and/or determining associated PK/PD parameters. The QSP model will use compartments to provide boundaries between regions of the subject's body where the activatable bispecific antibody is subject to different environments and consequently different reactions and/or reaction conditions. The activatable bispecific antibody can move between compartments via physical transport mechanisms such as diffusion, perfusion, and/or active transport. Further, even without such mass transport mechanisms, an activatable bispecific antibody's concentration in a compartment may change due to osmosis, degradation (extra- or intracellular), or other passive or active mechanism in which the antibody doesn't necessarily pass between compartments.

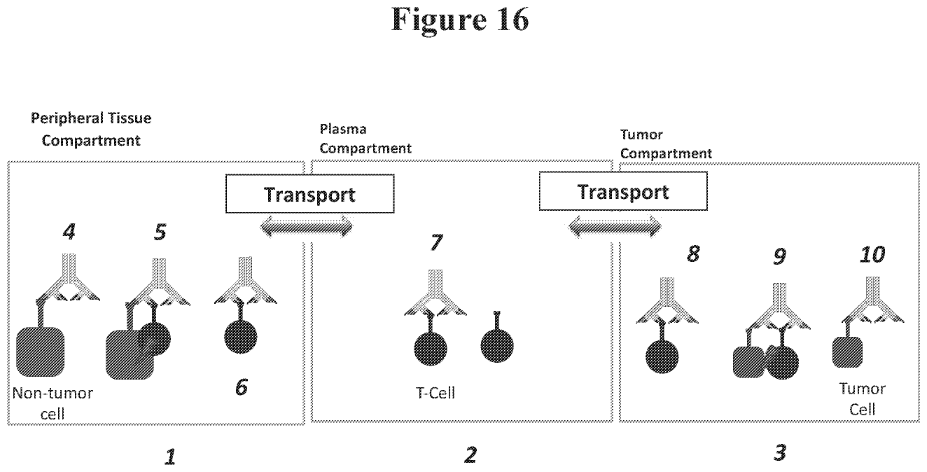

An example of a compartmental arrangement for a QSP model of the present disclosure of an activatable bispecific antibody is depicted in FIG. 16. The depicted arrangement includes peripheral tissue (item no. 1), plasma (item no. 2), and tumor (item no. 3) compartments. Concurrently, all forms/species of an activatable bispecific antibody can distribute to the plasma, peripheral, and tumor compartments. In the peripheral and tumor compartments, a subset of activatable or activated bispecific antibodies may engage in monovalent or bivalent binding with one or both of its targets, depending upon the number of breathing or cleaved prodomains at each binding site. For example, in the peripheral tissue compartment, an activated bispecific antibody can bind with the target antigen (e.g., EGFR) on a non-tumor cell as depicted (item nos. 4 and 5). Similarly, in the tumor compartment, an activated bispecific antibody can bind with the target antigen (e.g., EGFR) on a tumor cell (item nos. 9 and 10). In another feature of the QSP model of the present disclosure, an activated bispecific antibody can bind with the T-cell specific antigen (e.g., CD3) on a T-cell as depicted (item nos. 5, 6, 7, 8, and 9) in any of the three depicted compartments. In the QSP model of the present disclosure, when an activated bispecific antibody binds both a T-cell and a cell with the target antigen to form a trimeric complex (item nos. 5 and 9), then the cell with the target antigen can be killed by the T-cell. This killing of the target can be governed by the rate constant kₖᵢₗₗ.

In certain exemplary human QSP models of the present disclosure relating to activatable T-cell bispecific antibodies, the model may include certain exemplary assumptions. Based on the type of tumor, the T-cell distribution, etc., human QSP models may assume different target antigen expression levels in different cell types, different T-cell distributions in different compartments, different cleavage rates for different types of prodomains, etc. In some examples, cells in the tumor and non-tumor compartments express the target antigen at the same expression levels (e.g., 75,000 EGFR target antigens per cell). In other embodiments, the target expression level of the target antigen can be higher on tumor cells in the tumor compartment, reflecting indications where tumor cells overexpress the target antigen. In some embodiments, the QSP model of the present disclosure assumes that T-cells are populated equally in each compartment, and do not increase or decrease during the course of the model. In some embodiments, the QSP model of the present disclosure assumes that there are an equal number of target antigen-expressing cells and T-cells in each compartment (i.e. an E:T ratio of 1). In some embodiments, the QSP model of the present disclosure assumes the target antigen-expressing cells double in number at a defined and presume rate in the tumor compartment, but not in the non-tumor compartments. In some embodiments, the QSP model of the present disclosure assumes that the cleavage rate constant (k_{cleave}) for all prodomains on the activatable bispecific antibody are the same, such that the cleavage rate constant for the prodomain for the T-cell specific binding portion (e.g., the anti-CD3) is the same as that for the prodomain for the target antigen specific binding portion (e.g., the anti-EGFR). In some embodiments, the QSP model of the present disclosure assumes the size of tumor compartment (e.g., 10 mL) and average volume of the target antigen-expressing cell (e.g., 2×10e-12 L/cell) based on target-mediated drug distribution estimations in cynomolgus monkeys.

### Example 10: QSP Modeling of the Dosage of Activatable T-Cell Bispecific Antibodies in Humans

In this example, the human QSP model of the present disclosure was used to estimate the biologically effective dose (BED) of an activatable T-cell bispecific antibody that, when both its AB1 and AB2 are activated, is capable of specifically binding to both CD3 receptor on T-cells and EGFR on target cells, such as tumor cells. When the activated T-cell bispecific antibody binds both cell types simultaneously to form a trimer complex of the bispecific antibody, the target cell, and the T-cell, killing of the target cell by the T-cell can be effected by defined first-order rate constant (kₖᵢₗₗ).

In this example, a QSP model of the present disclosure was based on known or derived parameters such as those listed in Table 5. The modeled activatable bispecific antibody was an anti-CD3 and anti-EGFR activatable bispecific antibody. In this example, the QSP model included a parameter for the doubling time of the EGFR-expressing tumor cells in the tumor compartment. The BED for the activatable bispecific antibody was based on a model to predict the dosage of the activatable bispecific antibody that would result in stasis of the tumor cells in the tumor compartment resulting from tumor cell killing resulting from formation of the trimer complex.

In this exemplary QSP model of the present disclosure, after single Q3W dosage a BED of 0.170 mg/kg of the activatable bispecific antibody was predicted that would result in cytostatis of the tumor cells in the tumor compartment, as well as resulting in a serum Cmax of 18.3 nM. When eight (8) Q3W doses of the activatable bispecific antibody were modeled, a lower BED of 0.086 mg/kg and serum Cₘₐₓ of 9.3 mg/kg was predicted, as the multiple dosages would result in a steady-state level of the therapeutic drug and the resulting trimer complex, which was predicted to result in more effective killing of the tumor cells.

TABLE 5
Exemplary Parameters in Human QSP Model for Activatable T-Cell Bispecific Antibody

| | Cyno PD- | |
|---|---|---|
| Parameter (Unit) | 1 mAb | Notes |
| Human Body Wt. (kg) | 77 | Based on typical or avg. values |
| Human Surface Area (m²) | 2.1 | Based on typical or avg. values |
| Central vol. Vc (mL) | 3300 | Based on typical or avg. values |
| Peripheral vol., Vp (mL) | 3300 | Fixed based on peripheral volume |
| Elimination rate constant | 1.37e−6 | Allometrically scaled from experimental cyno |
| (central & peripheral) | | value based on relative body weights. |
| kₑₗ (sec⁻¹) | | |
| K₁₂ (sec⁻¹) | 4.28e−4 | Allometrically scaled from experimental cyno |
| | | value based on relative body weights. |
| K₂₁ (sec⁻¹) | 4.28e−4 | Allometrically scaled from experimental cyno |
| | | value based on relative body weights. |
| Human T_{1/2} of the Activatable | 5.86 | |
| Antibody | | |
| T_{dist} central-peripheral (hr) | 0.2256 | Computed from scaling |
| T_{dist} central-tumor (hr) | 0.2256 | Assume same as T_{dist} central-peripheral |
| P_{dist} central-peripheral | 1 | Computed from scaling |
| partition coefficient | | |
| P_{dist} central-tumor | 0.21 | Assume same as atezolizumab (Deng, et al.) |
| partition coefficient | | |
| Tumor vol., Vt (mL) | 10 | In the range of breast tumor ER/PR+ |
| Tumor cellularity (%) | 80 | Approximate for solid tumor |
| T_{1/2} of CD3 on T-cells (hr) | 10.5 | Literature reference |
| T_{1/2} of EGFR on target cells | 24 | Literature reference |
| (hr) | | |
| CD3 per cell | 53,000 | Literature reference |
| EGFR per cell | 75,000 | Literature reference |
| Conc. of EGFR+ cells (nM) | 8.3e−4 (tumor) | Tumor concentration calculated based on tumor |
| | 2.1e−4 (non-tumor) | volume and avg. cell size (2e−12 L) |
| | | Non-tumor concentration based on cyno TMDD PK |
| | | fit |
| Conc. of T-cells (nM) | 8.3e−4 (tumor) | Assumed EGFR+:T-cell ratio of 1. |
| | 2.1e−4 (non-tumor) | |
| Kd Ab:CD3 (nM) | 7.21 | Experimental data of in vitro binding to Jurkat |
| Kd monovalent (nM) | 14.42 | cells |
| Kd Ab:EGFR (nM) | 0.1392 | Experimental data of in vitro binding to HT29 |
| Kd monovalent (nM) | 0.2784 | cells |
| CD3 prodomain (k_{close}/kₒₚₑₙ) | 9.687 | Ratio of activatable T-cell bispecific Ab to |
| | | activated T-cell bispecific Ab |
| EGFR prodomain (k_{close}/kₒₚₑₙ) | 1.599 | Ratio of activatable T-cell bispecific Ab to |
| | | activated T-cell bispecific Ab |
| K_{cleave} (sec⁻¹) | 8.18.e−8 | Based on anti-PD-1 activatable antibody k_{cleave} |
| Cleavage ratio (tumor/non- | 100 | Assumed value |
| tumor) | | |
| Kₖᵢₗₗ trimer complex (sec⁻¹) | 2.38e−8 | Fit to experimental mouse data of tumor growth |
| | | inhibition |
| Tumor doubling time (days) | 69.1 | Based on TNBC |

### Other Embodiments

The invention may be defined by reference to the following illustrative clauses:

1. A method for modeling an activatable antibody between a plurality of compartments, the method comprising:

(A) providing a plurality of compartments wherein each compartment includes one of a plurality of species of activatable antibody, the plurality of species comprising:
(a) a first species comprising a quantity of uncleaved activatable antibody,
(b) a second species comprising a quantity of partially cleaved activatable antibody,
(c) a third species comprising a quantity of fully cleaved activatable antibody, and
(d) a fourth species comprising a quantity of fully unmasked partially cleaved activatable antibody,

(B) providing a rate expression between each of pair of compartments selected from the plurality of compartments, wherein each rate expression reflects the rate of conversion between the species in the given pair of compartments; and

(C) determining the distribution of species between the plurality of compartments,

(D) wherein the activatable antibody comprises:
(i) an antibody or an antigen binding fragment thereof (AB) that specifically binds to a target,
(ii) a masking moiety (MM) coupled to the AB that inhibits the binding of the AB of the activatable antibody in an uncleaved state to the target, wherein the MM of the activatable antibody in an uncleaved state interferes with specific binding of the AB to the target, and
(iii) a cleavable moiety (CM) coupled to the AB, wherein the CM is a polypeptide that functions as a substrate for a cleaving agent, whereby cleavage of the uncleaved activatable antibody in the CM results in an activated activatable antibody, and
(iv) wherein the activatable antibody in the uncleaved state has the structural arrangement from N-terminus to C-terminus as follows: MM-CM-AB or AB-CM-MM2.

1A. The method of clause 1, wherein the plurality of species comprises:

(f) a fifth species comprising a quantity of a partially unmasked uncleaved activatable antibody, and (g) a sixth species comprising a quantity of fully unmasked uncleaved activatable antibody.

2. The method of clause 1 or 1A comprising:

(E) providing a plurality of physiological compartments comprising (a) a first physiological compartment comprising a quantity of activatable antibody species in a vascular compartment, (b) a second physiological compartment comprising a quantity of activatable antibody species in a peripheral tissue compartment, and (c) a third physiological compartment comprising a quantity of activatable antibody species in a tumor compartment,

(F) providing a rate expression between each of pair of physiological compartments selected from the plurality of physiological compartments, wherein each rate expression reflects the rate of transport of activatable antibody species between the given pair of physiological compartments; and

(G) providing a rate expression for each physiological compartment reflecting the rate of elimination of activatable antibody species from the given physiological compartment.

3. The method of clause 1 or 1A comprising:

(E) providing a plurality of physiological compartments comprising (a) a first physiological compartment comprising a quantity of activatable antibody species in a plasma compartment, (b) a second physiological compartment comprising a quantity of activatable antibody species in a peripheral tissue compartment, and (c) a third physiological compartment comprising a quantity of activatable antibody species in a tumor compartment,

(F) providing a equilibrium coefficient between each of pair of physiological compartments selected from the plurality of physiological compartments, wherein each equilibrium coefficient reflects the ratio of activatable antibody species between the given pair of physiological compartments; and

(G) providing a rate expression for each physiological compartment reflecting the rate of elimination of activatable antibody species from the given physiological compartment.

4. The method of clause 1 or 1A comprising:

(E) providing a plurality of physiological compartments comprising

(g) a first physiological compartment comprising a quantity of activatable antibody species in a plasma compartment, (h) a second physiological compartment comprising a quantity of activatable antibody species in a peripheral tissue compartment, and (i) a third physiological compartment comprising a quantity of activatable antibody species in a tumor compartment,

(F) providing a perfusion rate between each of pair of physiological compartments selected from the plurality of physiological compartments, wherein each perfusion rate reflects the rate of transport of activatable antibody species between the given pair of physiological compartments; and

(G) providing a rate expression for each physiological compartment reflecting the rate of elimination of activatable antibody species from the given physiological compartment.

5. The method of any one of clauses 1-4 comprising:
(E) providing in each of the second and third physiological compartments a quantity of the target,

wherein for each physiological compartment, a synthesis rate of the target and an endocytosis rate of the target is provided, and

wherein the quantity of the target in a given physiological compartment is based on the synthesis rate and the endocytosis rate in the given compartment.

6. The method of any one of clauses 2-5 comprising:

(a) providing in each of the second and third physiological compartments a monovalent target occupancy compartment and a bivalent target occupancy compartment,

wherein the monovalent target occupancy compartment includes a quantity of the species from the second, third, fourth, fifth, and sixth species compartments in the given physiological compartment that are bound to the target in the given physiological compartment,

wherein the bivalent target occupancy compartment includes a quantity of the species from the third and sixth species compartments in the given physiological compartment that are bound to the target in the given physiological compartment; and

(b) providing a first on-rate expression reflecting the rate of binding to the target of the first binding element of the activatable antibody species having a first unmasked antigen-binding site, (c) providing a second on-rate expression reflecting the rate of binding to the target of the first binding element of the activatable antibody species having a first masked and uncleaved antigen-binding site, (d) providing a third on-rate expression reflecting the rate of binding to the target of the first binding element of the activatable antibody species having a first cleaved antigen-binding site, (e) providing a fourth on-rate expression reflecting the rate of binding to the target of the second binding element of the activatable antibody species having a second unmasked antigen-binding site, (f) providing a fifth on-rate expression reflecting the rate of binding to the target of the second binding element of the activatable antibody species having a second masked and uncleaved antigen-binding site, (g) providing a sixth on-rate expression reflecting the rate of binding to the target of the second binding element of the activatable antibody species having a second cleaved antigen-binding site, (h) providing a first off-rate expression reflecting the rate of target release of the first or second binding element of the activatable antibody species having an unmasked antigen-binding site, (i) providing a second off-rate expression reflecting the rate of target release of the first or second binding element of the activatable antibody species having an masked and uncleaved antigen-binding site, and (j) providing a third off-rate expression reflecting the rate of target release of the first or second binding element of the activatable antibody species having a cleaved antigen-binding site.

6A. The method of any one of clauses 2-5 comprising:

(a) providing in each of the second and third physiological compartments a monovalent target occupancy compartment and a bivalent target occupancy compartment,

wherein the monovalent target occupancy compartment includes a quantity of the species from the second, third, fourth, fifth, and sixth species compartments in the given physiological compartment that are bound to the target in the given physiological compartment,

wherein the bivalent target occupancy compartment includes a quantity of the species from the third and sixth species compartments in the given physiological compartment that are bound to the target in the given physiological compartment; and

(b) providing a first equilibrium constant reflecting the binding between the target and the first binding element of the activatable antibody species having a first unmasked antigen-binding site, (c) providing a second equilibrium constant reflecting the binding between the target and the first binding element of the activatable antibody species having a first masked and uncleaved antigen-binding site, (d) providing a third equilibrium constant reflecting the binding between the target and the first binding element of the activatable antibody species having a first cleaved antigen-binding site, (e) providing a fourth equilibrium constant reflecting the binding between the target and the second binding element of the activatable antibody species having a second unmasked antigen-binding site, (f) providing a fifth equilibrium constant reflecting the binding between the target and the second binding element of the activatable antibody species having a second masked and uncleaved antigen-binding site, and (g) providing a sixth equilibrium constant reflecting the binding between the target and the second binding element of the activatable antibody species having a second cleaved antigen-binding site, (h) providing a first off-rate expression reflecting the rate of target release of the first or second binding element of the activatable antibody species having an unmasked antigen-binding site.

7. The method of any one of clauses 2-6A comprising determining the distribution of species between the plurality of physiological compartments.

8. The method of any one of clauses 6, 6A, and 7 comprising determining the distribution of species between the plurality of target occupancy compartments.

9. The method of any one of clauses 1-8, wherein the step of determining the distribution of species between the plurality of species compartments comprises determining the distribution at a plurality of time points.

10. The method of any one of clauses 2-9 comprising determining the distribution of species between the plurality of physiological compartments comprises determining the distribution at a plurality of time points.

11. The method of any one of clauses 6-10 comprising determining the distribution of species between the plurality of target occupancy compartments comprises determining the distribution at a plurality of time points.

12. The method of any one of clauses 6-11, wherein the step of determining the distribution of species between the plurality of species compartments comprises determining the distribution at a plurality of time points.

13. The method of any one of clauses 1-12, wherein the rate constant of the rate expression between any given pair of species compartments is a first-order rate constant.

14. The method of any one of clauses 1-13, wherein (a) the rate expression of conversion from the second species compartment to the first species compartment is zero, (b) the rate expression of conversion from the third species compartment to the second species compartment is zero, (c) the rate expression of conversion from the second species compartment to the fourth species compartment is zero, (d) the rate expression of conversion from the third species compartment to the sixth species compartment is zero, or (e) the rate expression of conversion from the sixth species compartment to the fifth species compartment is zero.

15. A method of calibrating a model of the distribution of species of activatable antibody in a subject comprising: determining the distribution of species of activatable antibody at a plurality of time points according to the modeling method of any one of clauses 9 to 12; comparing the distribution of species of activatable antibody at the plurality of time points in the model to an observed distribution of species of activatable antibody at a plurality of time points in a subject; and modifying one or more parameters in the model, whereby the modified model has a higher concordance to the observed distribution of species.

16. A method of determining a dosage range of an activatable antibody in a subject comprising: determining the distribution of species of activatable antibody at a plurality of time points for given dosages of activatable antibody according to the modeling method of any one of clauses 9 to 12; and selecting the given dosages for administration to the subject based the modeled distribution of the activatable antibody species for each given dosage.

17. The method of clause 16, wherein the selection of the given dosages is based on the modeled plasma concentration of the activatable antibody species for a given dosage.

### BIOLOGICAL SEQUENCE LISTING

The sequence listing is shown in Table A below.

TABLE A
Sequence Listing

| SEQ | | |
|---|---|---|
| ID | | |
| NO: | NAME | SEQUENCE |
| 1 | CM | LSGRSDNH |
| 2 | CM | TGRGPSWV |
| 3 | CM | PLTGRSGG |
| 4 | CM | TARGPSFK |
| 5 | CM | NTLSGRSENHSG |
| 6 | CM | NTLSGRSGNHGS |
| 7 | CM | TSTSGRSANPRG |
| 8 | CM | TSGRSANP |
| 9 | CM | VHMPLGFLGP |
| 10 | CM | AVGLLAPP |
| 11 | CM | AQNLLGMV |
| 12 | CM | QNQALRMA |
| 13 | CM | LAAPLGLL |
| 14 | CM | STFPFGMF |
| 15 | CM | ISSGLLSS |
| 16 | CM | PAGLWLDP |
| 17 | CM | VAGRSMRP |
| 18 | CM | VVPEGRRS |
| 19 | CM | ILPRSPAF |
| 20 | CM | MVLGRSLL |
| 21 | CM | QGRAITFI |
| 22 | CM | SPRSIMLA |
| 23 | CM | SMLRSMPL |
| 24 | CM | ISSGLLSGRSDNH |
| 25 | CM | AVGLLAPPGGLSGRSDN |
| 26 | CM | ISSGLLSSGGSGGSLSGRSDNH |
| 27 | CM | LSGRSGNH |
| 28 | CM | SGRSANPRG |
| 29 | CM | LSGRSDDH |
| 30 | CM | LSGRSDIH |
| 31 | CM | LSGRSDQH |
| 32 | CM | LSGRSDTH |
| 33 | CM | LSGRSDYH |
| 34 | CM | LSGRSDNP |
| 35 | CM | LSGRSANP |
| 36 | CM | LSGRSANI |
| 37 | CM | LSGRSDNI |
| 38 | CM | MIAPVAYR |
| 39 | CM | RPSPMWAY |
| 40 | CM | MATPRPMR |
| 41 | CM | FRLLDWQW |
| 42 | CM | ISSGL |
| 43 | CM | ISSGLLS |
| 44 | CM | ISSGLL |
| 45 | CM | ISSGLLSGRSANPRG |
| 46 | CM | AVGLLAPPTSGRSANPRG |
| 47 | CM | AVGLLAPPSGRSANPRG |
| 48 | CM | ISSGLLSGRSDDH |
| 49 | CM | ISSGLLSGRSDIH |
| 50 | CM | ISSGLLSGRSDQH |
| 51 | CM | ISSGLLSGRSDTH |
| 52 | CM | ISSGLLSGRSDYH |
| 53 | CM | ISSGLLSGRSDNP |
| 54 | CM | ISSGLLSGRSANP |
| 55 | CM | ISSGLLSGRSANI |
| 56 | CM | AVGLLAPPGGLSGRSDDH |
| 57 | CM | AVGLLAPPGGLSGRSDIH |
| 58 | CM | AVGLLAPPGGLSGRSDQH |
| 59 | CM | AVGLLAPPGGLSGRSDTH |
| 60 | CM | AVGLLAPPGGLSGRSDYH |
| 61 | CM | AVGLLAPPGGLSGRSDNP |
| 62 | CM | AVGLLAPPGGLSGRSANP |
| 63 | CM | AVGLLAPPGGLSGRSANI |
| 64 | CM | ISSGLLSGRSDNI |
| 65 | CM | AVGLLAPPGGLSGRSDNI |
| 66 | CM | GLSGRSDNHGGAVGLLAPP |
| 67 | CM | GLSGRSDNHGGVHMPLGFLGP |
| 68 | Linker | GSGGS |
| 69 | Linker | GGGS |
| 70 | Linker | GGSG |
| 71 | Linker | GGSGG |
| 72 | Linker | GSGSG |
| 73 | Linker | GSGGG |
| 74 | Linker | GGGSG |
| 75 | Linker | GSSSG |
| 76 | Linker | GSSGGSGGSGGSG |
| 77 | Linker | GSSGGSGGSGG |
| 78 | Linker | GSSGGSGGSGGS |
| 79 | Linker | GSSGGSGGSGGSGGGS |
| 80 | Linker | GSSGGSGGSG |
| 81 | Linker | GSSGGSGGSGS |
| 82 | Linker | GGGS |
| 83 | Linker | GSSGT |
| 84 | Linker | GSSG |
| 85 | MM | YLCQRHPLALKYCTN |
| 86 | MM | PLCVPTQLLRSCYNY |
| 87 | MM | AVCHPLANVETQCLD |
| 88 | MM | PHCHPLFNNTYCYRH |
| 89 | MM | PLCRPIELLASCPMK |
| 90 | MM | GAACVSAWGFFCECC |
| 91 | MM | DCAKDILHLMPHCSM |
| 92 | MM | NTCMHPLLLQGCKTY |
| 93 | MM | YLGCLLYAGPGCEGG |
| 94 | MM | ARCPHPLLLSICENN |
| 95 | MM | ELCPHPLPFGFCNNY |
| 96 | MM | ALYCHPPYIRCEEMT |
| 97 | MM | TSLCHPVMIMYCKTG |
| 98 | MM | PLCHPLEQASWCNMD |
| 99 | MM | PHPCPRTGSRMCHFS |
| 100 | MM | SGCRHPLPLKACGTN |
| 101 | MM | GLCHPIRLHNTQCTI |
| 102 | MM | KCMHPLNLHNINCNH |
| 103 | MM | PICHPLREFMNTCFK |
| 104 | MM | NCHPLDVVGWLGCMK |
| 105 | MM | YNNVCHPLFCSQHTY |
| 106 | MM | TFCHPLFSLNYCGHK |
| 107 | MM | FCHPLTLSNNKQCNR |
| 108 | MM | LSHCAVLLLRVCSGS |
| 109 | MM | KIHCHPLRLGTCLVG |
| 110 | MM | ETCAHPLDMRMCRHN |
| 111 | MM | PLCYPLILMSSCWLG |
| 112 | MM | YGICHPAPDLPCMQI |
| 113 | MM | TACHPLYNVEHLCEI |
| 114 | MM | TACNKSVCVAGCCLL |
| 115 | MM | LHPLCSYMKSCMKNN |
| 116 | MM | THCHCMVYFCPCRWS |
| 117 | MM | PKCPHPLHLANCYAS |
| 118 | MM | KTCYHPTPVIAXNSY |
| 119 | MM | AKCLPPLIQYCRCIK |
| 120 | MM | HACQHPLQLHTCKHN |
| 121 | MM | LCHPLVLSAWESCSN |
| 122 | MM | WPLCSFGKSFCAQNA |
| 123 | MM | ECQSFEHFLTNNCHS |
| 124 | MM | SCKHPLVMPNLKCTR |
| 125 | MM | YPCHPLQLSIPHCTK |
| 126 | MM | ICHPLTHTMEYMCMN |
| 127 | MM | TLCHPLTFSVPTCTN |
| 128 | MM | PLCQPNRLLQACGNT |
| 129 | MM | TLCRHPLALDGCQNN |
| 130 | MM | QPMCYQPAHPLCNTI |
| 131 | MM | SNCHPLLFQHYHCML |
| 132 | MM | EKCYHPLTLAHCQNH |
| 134 | MM | NKCFVHPLAMPNCNS |
| 134 | MM | VNNCLLMTRAHCTSY |
| 135 | MM | LPCWAFAVNPLHCGD |
| 136 | MM | VNNCLLMTRAHCTSY |
| 137 | MM | SSCPHPLGLTGCNDK |
| 138 | MM | NKCFVHPLAMPNCNS |
| 139 | MM | FVGCHSVYVSGCLRA |
| 140 | MM | NMCHPPHNIYSICNM |
| 141 | MM | LTCHLLPGLTLH-TK |
| 142 | MM | RTCHPLPGLTLHCTK |
| 143 | MM | HPLCFESMKNCFPNY |
| 144 | MM | TTCHPLSFTHNYCIT |
| 145 | MM | RDCGFDAVRADCLFG |
| 146 | MM | RICSTHPLIMPQCNY |
| 147 | MM | MKCHPLQLTGNTCSM |
| 148 | MM | SGCPHPLQLITCSTA |
| 149 | MM | KCFPAFHDGPLACAS |
| 150 | MM | LKCQHPLPMSHCQPQ |
| 151 | MM | AFCGFSVIHPLCSGA |
| 152 | MM | SVHCAVLKLDGCLGW |
| 153 | MM | TLPCHPIMVLGCTPM |
| 154 | MM | HYPCMKYNPLNCSMS |
| 155 | MM | LKCPHPLSLNGCTLK |
| 156 | MM | VYSCMANNPLDCFTQ |
| 157 | MM | PICHPLVTLMSYCNK |
| 158 | MM | DWCSFWAGQSVWCTS |
| 159 | MM | STCHPLTPFHDKCRY |
| 160 | MM | PVCPPLVTLMSYCNK |
| 161 | MM | STCHPLPTLMPYCNS |
| 162 | MM | FPLCGIGPAFCDTTV |
| 163 | MM | PTCHPLVLSVPCPKI |
| 164 | MM | GPLCDYFVFYSCRGS |
| 165 | MM | HTCYHPLKLGQCEMF |
| 166 | MM | RTCIHPLPLHQCHKP |
| 167 | MM | ACHPINFNSIVYCNN |
| 168 | MM | SHPCSVVNLPGCEPD |
| 169 | MM | LCHPLVLSAWESCSS |
| 170 | MM | LCaPLVLSAWESCSS |
| 171 | MM | LCHaLVLSAWESCSS |
| 172 | MM | LCHPaVLSAWESCSS |
| 173 | MM | LCHPLaLSAWESCSS |
| 174 | MM | LCHPLVaSAWESCSS |
| 175 | MM | LCHPLVLSAaESCSS |
| 176 | MM | LCHPLVLSAWaSCSS |
| 177 | MM | CHPLVLSAWESC |
| 178 | MM | HPLVL |
| 180 | MM | LEGWCLHPLCLWGAG |
| 181 | MM | LEGaCLHPLCLWGAG |
| 182 | MM | LEGWCaHPLCLWGAG |
| 183 | MM | LEGWCLaPLCLWGAG |
| 184 | MM | LEGWCLHaLCLWGAG |
| 185 | MM | LEGWCLHPaCLWGAG |
| 186 | MM | LEGWCLHPLCaWGAG |
| 187 | MM | LEGWCLHPLCLaGAG |
| 188 | MM | CLHPLC |
| 189 | Spacer | QGQSGS |
| 190 | Spacer | GQSGS |
| 191 | Spacer | QSGS |
| 194 | Spacer | QGQSGQG |
| 195 | Spacer | GQSGQG |
| 196 | Spacer | QSGQG |
| 197 | Spacer | SGQG |
| 200 | Spacer | QGQSGQ |
| 201 | Spacer | GQSGQ |
| 202 | Spacer | QSGQ |
| 205 | VH#1 domain of | QITLKESGPTLVKPTQTLTLTCTFS |
| | anti-CD166 | GFSLSTYGMGVGWIRQPPGKALEWL |
| | antibody | ANIWWSEDKHYSPSLKSRLTITKDT |
| | | SKNQWLTMTNMDPVDTATYYCVQID |
| | | YGNDYAETYWGQGTLVTVSS |
| 206 | VH#2 domain of | QITLKESGPTLVKPTQTLTLTCTFS |
| | anti-CD166 | GFSLSTYGMGVGWIRQPPGKALEWL |
| | antibody | ANIWWSEDKHYSPSLKSRLTITKDT |
| | | SKNQWLTITNVDPVDTATYYCVQID |
| | | YGNDYAFTYWGQGTLVTVSS |
| 207 | VL#1 domain of | DIVMTQSPLSLPVTPGEPASISCRS |
| | anti-CD166 | SKSLLHSNGITYLYWYLQKPGQSPQ |
| | antibody | LLIYQMSNLASGVPDRFSGSGSGTD |
| | | FTLKISRVEAEDVGVYYCAQNLELP |
| | | YTFGQGTKLEIK |
| 208 | VL#2 domain of | DIVMTQSPLSLPVTPGEPASISCRS |
| | anti-CD166 | SKSLLHSNGITYLYWYLQKPGQSPQ |
| | antibody | LLIYQMSNLASGVPDRFSSSGSGTD |
| | | FTLKISRVEAEDVGVYYCAQNLELP |
| | | YTFGQGTKLEIK |
| 209 | VL#3 domain of | DIVMTQSPLSLPVTPGEPASISCRS |
| | anti-CD166 | SQSLLHSNGITYLYWYLQKPGQSPQ |
| | antibody | LLIYQMSNRASGVPDRFSGSGSGTD |
| | | FTLKISRVEAEDVGVYYCAQNLELP |
| | | YTFGQGTKLEIK |
| 210 | VL#4 domain of | DIVMTQSPLSLPVTPGEPASISCRS |
| | anti-CD166 | SQSLLHSNGITYLYWYLQKPGQSPQ |
| | antibody | QLLIYQMSNRASGVPDRFSSSGSGT |
| | | DFTLKISRVEAEDVGVYYCANLELP |
| | | YTFGQGTKLEIK |
| 211 | VL (M2,S1) | LCHPAVLSAWESCSSGGGSSGGSIS |
| | domain of anti- | SGLLSGRSDNHGGGSDIVMTQSPLS |
| | CD166 | LPVTPGEPASISCRSSKSLLHSNGI |
| | activatable | TYLYWYLQKPGQSPQLLIYQMSNLA |
| | antibody | SGVPDRFSGSGSGTDFTLKISRVEA |
| | | EDVGVYYCAQNLELPYTFGQGTKLE |
| | | IK |
| 212 | VL (Ml,S1) | LCHPLVASAWESCSSGGGSSGGSIS |
| | domain of anti- | SGLLSGRSDNHGGGSDIVMTQSPLS |
| | CD166 | LPVTPGEPASISCRSSKSLLHSNGI |
| | activatable | TYLYWYLQKPGQSPQLLIYQMSNLA |
| | antibody | SGVPDRFSGSGSGTDFTLKISRVEA |
| | | EDVGVYYCAQNLELPYTFGQGTKLE |
| | | IK |
| 213 | VL (M2,S2) | LCHPAVLSAWESCSSGGGSSGGSAV |
| | domain of anti- | GLLAPPGGLSGRSDNHGGSDIVMTQ |
| | CD166 | SPLSLPVTPGEPASISCRSSKSLLH |
| | activatable | SNGITYLYWYLQKPGQSPQLLIYQM |
| | antibody | SNLASGVPDRFSGSGSGTDFTLKIS |
| | | RVEAEDVGVYYCAQNLELPYTFGQG |
| | | TKLEIK |
| 214 | VL (Ml,S2) | LCHPLVASAWESCSSGGGSSGGSAV |
| | domain of anti- | GLLAPPGGLSGRSDNHGGSDIVMTQ |
| | CD166 | SPLSLPVTPGEPASISCRSSKSLLH |
| | act ivatable | SNGITYLYWYLQKPGQSPQLLIYQM |
| | antibody | SNLASGVPDRFSGSGSGTDFTLKIS |
| | | RVEAEDVGVYYCAQNLELPYTFGQG |
| | | TKLEIK |
| 215 | VL (M2,0) | LCHPAVLSAWESCSSGGGSSGGSGG |
| | domain of anti- | GSGGGSGGSDIVMTQSPLSLPVTPG |
| | CD166 | EPASISCRSSKSLLHSNGITYLYWY |
| | activatable | LQKPGQSPQLLIYQMSNLASGVPDR |
| | antibody | FSGSGSGTDFTLKISRVEAEDVGVY |
| | | YCAQNLELPYTFGQGTKLEIK |
| 216 | Heavy chain of | QITLKESGPTLVKPTQTLTLTCTFS |
| | anti-CD166 | GFSLSTYGMGVGWIRQPPGKALEWL |
| | activatable | ANIWWSEDKHYSPSLKSRLTITKDT |
| | antibody | SKNQWLTITNVDPVDTATYYCVQID |
| | | YGNDYAFTYWGQGTLVTVSSASTKG |
| | | PSVFPLAPSSKSTSGGTAALGCLVK |
| | | DYFPEPVTVSWNSGALTSGVHTFPA |
| | | VLQSSGLYSLSSWTVPSSSLGTQTY |
| | | ICNVNHKPSNTKVDKKVEPKSCDKT |
| | | HTCPPCPAPELLGGPSVFLFPPKPK |
| | | DTLMISRTPEVTCVWDVSHEDPEVK |
| | | FNWYVDGVEVHNAKTKPREEQYNST |
| | | YRVVSVLTVLHQDWLNGKEYKCKVS |
| | | NKALPAPIEKTISKAKGQPREPQVY |
| | | TLPPSREEMTKNQVSLTCLVKGFYP |
| | | SDIAVEWESNGQPENNYKTTPPVLD |
| | | SDGSFFLYSKLTVDKSRWQQGNVFS |
| | | CSVMHEALHNHYTQKSLSLSPGK |
| 217 | Light chain | LCHPAVLSAWESCSSGGGSSGGSIS |
| | (M2, S1) of | SGLLSGRSDNHGGGSDIVMTQSPLS |
| | anti-CD166 | LPVTPGEPASISCRSSKSLLHSNGI |
| | activatable | TYLYWYLQKPGQSPQLLIYQMSNLA |
| | antibody | SGVPDRFSGSGSGTDFTLKISRVEA |
| | | EDVGVYYCAQNLELPYTFGQGTKLE |
| | | IKRTVAAPSVFIFPPSDEQLKSGTA |
| | | SVVCLLNNFYPREAKVQWKVDNALQ |
| | | SGNSQESVTEQDSKDSTYSLSSTLT |
| | | LSKADYEKHKVYACEVTHQGLSSPV |
| | | TKSFNRGEC |
| 218 | Light chain | LCHPLVASAWESCSSGGGSSGGSIS |
| | M1, S1) of anti- | SGLLSGRSDNHGGGSDIVMTQSPLS |
| | CD166 | LPVTPGEPASISCRSSKSLLHSNGI |
| | activatable | TYLYWYLQKPGQSPQLLIYQMSNLA |
| | antibody | SGVPDRFSGSGSGTDFTLKISRVEA |
| | | EDVGVYYCAQNLELPYTFGQGTKLE |
| | | IKRTVAAPSVFIFPPSDEQLKSGTA |
| | | SWCLLNNFYPREAKVQWKVDNALQS |
| | | GNSQESVTEQDSKDSTYSLSSTLTL |
| | | SKADYEKHKVYACEVTHQGLSSPVT |
| | | KSFNRGEC |
| 219 | Light chain | LCHPAVLSAWESCSSGGGSSGGSAV |
| | (M2, S2) of | GLLAPPGGLSGRSDNHGGSDIVMTQ |
| | anti-CD166 | SPLSLPVTPGEPASISCRSSKSLLH |
| | activatable | SNGITYLYWYLQKPGQSPQLLIYQM |
| | antibody | SNLASGVPDRFSGSGSGTDFTLKIS |
| | | RVEAEDVGVYYCAQNLELPYTFGQG |
| | | TKLEIKRTVAAPSVFIFPPSDEQLK |
| | | SGTASVVCLLNNFYPREAKVQWKVD |
| | | NALQSGNSQESVTEQDSKDSTYSLS |
| | | STLTLSKADYEKHKVYACEVTHQGL |
| | | SSPVTKSFNRGEC |
| 220 | Light chain | LCHPLVASAWESCSSGGGSSGGSAV |
| | (M1, S2) of | GLLAPPGGLSGRSDNHGGSDIVMTQ |
| | anti-CD166 | SPLSLPVTPGEPASISCRSSKSLLH |
| | activatable | SNGITYLYWYLQKPGQSPQLLIYQM |
| | antibody | SNLASGVPDRFSGSGSGTDFTLKIS |
| | | RVEAEDVGVYYCAQNLELPYTFGQG |
| | | TKLEIKRTVAAPSVFIFPPSDEQLK |
| | | SGTASVVCLLNNFYPREAKVQWKVD |
| | | NALQSGNSQESVTEQDSKDSTYSLS |
| | | STLTLSKADYEKHKVYACEVTHQGL |
| | | SSPVTKSFNRGEC |
| 221 | Light chain | LCHPLVASAWESCSSGGGSSGGSGG |
| | (M1, 0) of anti- | GSGGGSGGSDIVMTQSPLSLPVTPG |
| | CD166 | EPASISCRSSKSLLHSNGITYLYWY |
| | activatable | LQKPGQSPQLLIYQMSNLASGVPDR |
| | antibody | FSGSGSGTDFTLKISRVEAEDVGVY |
| | | YCAQNLELPYTFGQGTKLEIKRTVA |
| | | APSVFIFPPSDEQLKSGTASWCLLN |
| | | NFYPREAKVQWKVDNALQSGNSQES |
| | | VTEQDSKDSTYSLSSTLTLSKADYE |
| | | KHKVYACEVTHQGLSSPVTKSFNRG |
| | | EC |
| 222 | Heavy chain of | QITLKESGPTLVKPTQTLTLTCTFS |
| | anti-CD166 | GFSLSTYGMGVGWIRQPPGKALEWL |
| | activatable | ANIWWSEDKHYSPSLKSRLTITKDT |
| | antibody | SKNQWLTITNVDPVDTATYYCVQID |
| | | YGNDYAFTYWGQGTLVTVSSASTKG |
| | | PSVFPLAPSSKSTSGGTAALGCLVK |
| | | DYFPEPVTVSWNSGALTSGVHTFPA |
| | | VLQSSGLYSLSSWTVPSSSLGTQTY |
| | | ICNVNHKPSNTKVDKKVEPKSCDKT |
| | | HTCPPCPAPELLGGPSVFLFPPKPK |
| | | DTLMISRTPEVTCWVDVSHEDPEVK |
| | | FNWYVDGVEVHNAKTKPREEQYNST |
| | | YRVVSVLTVLHQDWLNGKEYKCKVS |
| | | NKALPAPIEKTISKAKGQPREPQVY |
| | | TLPPSREEMTKNQVSLTCLVKGFYP |
| | | SDIAVEWESNGQPENNYKTTPPVLD |
| | | SDGSFFLYSKLTVDKSRWQQGNVFS |
| | | CSVMHEALHNHYTQKSLSLSPGK |
| 223 | Linker | GGGSSGGSGGSGG |
| 224 | Heavy chain of | EVQLLESGGGLVQPGGSLRLSCAAS |
| | anti-PD-L1 | GFTFSSYAMSWVRQAPGKGLEWVSS |
| | activatable | IWRNGIVTVYADSVKGRFTISRDNS |
| | antibody | KNTLYLQMNSLRAEDTAVYYCAKWS |
| | | AAFDYWGQGTLVTVSSASTKGPSVF |
| | | PLAPCSRSTSESTAALGCLVKDYFP |
| | | EPVTVSWNSGALTSGVHTFPAVLQS |
| | | SGLYSLSSWTVPSSSLGTKTYTCNV |
| | | DHKPSNTKVDKRVESKYGPPCPPCP |
| | | APEFLGGPSVFLFPPKPKDTLMISR |
| | | TPEVTCVWDVSQEDPEVQFNWYVDG |
| | | LVEVHNAKTKPREEQFNSTYRWSVT |
| | | VLHQDWLNGKEYKCKVSNKGLPSSI |
| | | EKTISKAKGQPREPQVYTLPPSQEE |
| | | MTKNQVSLTCLVKGFYPSDIAVEWE |
| | | SNGQPENNYKTTPPVLDSDGSFFLY |
| | | SRLTVDKSRWQEGNVFSCSVMHEAL |
| | | HNHYTQKSLSLSLG |
| 225 | Light chain of | QGQSGSGIALCPSHFCQLPQTGGGS |
| | anti-PD-L1 | SGGSGGSGGISSGLLSGRSDNHGGS |
| | activatable | DIQMTQSPSSLSASVGDRVTITCRA |
| | antibody | SQSISSYLNWYQQKPGKAPKLLIYA |
| | | ASSLQSGVPSRFSGSGSGTDFTLTI |
| | | SSLQPEDFATYYCQQDNGYPSTFGG |
| | | GTKVEIKRTVAAPSVFIFPPSDEQL |
| | | KSGTASWCLLNNFYPREAKVQWKVD |
| | | NALQSGNSQESVTEQDSKDSTYSLS |
| | | STLTLSKADYEKHKVYACEVTHQGL |
| | | SSPVTKSFNRGEC |
| 226 | Heavy chain of | EVQLVESGGGLVQPGGSLRLSCAAS |
| | anti-PD-1 | GFTFSGYAMSWVRQAPGKGLEWVAY |
| | activatable | ISNSGGNAHYADSVKGRFTISRDNS |
| | antibody and | KNTLYLQMNSLRAEDTAVYYCTRED |
| | anti-PD-1 | YGTSPFVYWGQGTLVTVSSASTKGP |
| | antibody | SVFPLAPCSRSTSESTAALGCLVKD |
| | | YFPEPVTVSWNSGALTSGVHTFPAV |
| | | LQSSGLYSLSSWTVPSSSLGTKTYT |
| | | CNVDHKPSNTKVDKRVESKYGPPCP |
| | | PCPAPEFLGGPSVFLFPPKPKDTLM |
| | | ISRTPEVTCVWDVSQEDPEVQFNWY |
| | | VDGVEVHNAKTKPREEQFNSTYRVV |
| | | SVLTVLHQDWLNGKEYKCKVSNKGL |
| | | PSSIEKTISKAKGQPREPQVYTLPP |
| | | SQEEMTKNQVSLTCLVKGFYPSDIA |
| | | VEWESNGQPENNYKTTPPVLDSDGS |
| | | FFLYSRLTVDKSRWQEGNVFSCSVM |
| | | HEALHNHYTQKSLSLSLG |
| 227 | Light chain of | QGQSGQGTSYCSIEHYPCNTHHGGG |
| | anti-PD-1 | SSGGSISSGLLSGRSDNPGGGSDIQ |
| | activatable | LTQSPSSLSASVGDRVTITCRASES |
| | antibody | VDAYGISFMNWFQQKPGKAPKLLIY |
| | | AASNQGSGVPSRFSGSGSGTDFTLT |
| | | ISSMQPEDFATYYCQQSKDVPWTFG |
| | | QGTKLEIKRTVAAPSVFIFPPSDEQ |
| | | LKSGTASWCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| 228 | Light chain of | DIQLTQSPSSLSASVGDRVTITCRA |
| | anti-PD-1 | SESVDAYGISFMNWFQQKPGKAPKL |
| | antibody | LIYAASNQGSGVPSRFSGSGSGTDF |
| | | TLTISSMQPEDFATYYCQQSKDVPW |
| | | TFGQGTKLEIKRTVAAPSVFIFPPS |
| | | DEQLKSGTASWCLLNNFYPREAKVQ |
| | | WKVDNALQSGNSQESVTEQDSKDST |
| | | YSLSSTLTLSKADYEKHKVYACEVT |
| | | HQGLSSPVTKSFNRGEC |
| 229 | Linker | GGGSSGGS |

While the foregoing invention has been described in some detail for purposes of clarity and understanding, it will be clear to one skilled in the art from a reading of this disclosure that various changes in form and detail can be made without departing from the true scope of the invention. It is understood that the materials, examples, and embodiments described herein are for illustrative purposes only and not intended to be limiting and that various modifications or changes in light thereof will be suggested to persons skilled in the art and are to be included within the spirit and scope of the appended claims.

What is claimed is:

1. A method of preparing a quantitative systems pharmacology model for predicting the disposition of an activatable antibody administered to a subject, the method comprising:
(a) providing at least one relationship or parameter characterizing mass transfer of activatable antibody and/or activated antibody between a non-target compartment of the subject and a target compartment of the subject, wherein the target compartment comprises a target to which an antibody or an antigen binding fragment (AB) specifically binds,
wherein the activatable antibody comprises an AB and a prodomain, wherein the prodomain comprises a masking moiety (MM) and a cleavable moiety (CM), wherein the activatable antibody has a reduced binding affinity to the target compared to the AB,
wherein the activated antibody comprises an AB that includes at least one prodomain that no longer masks the AB or lacks at least one prodomain relative to the activatable antibody, wherein the activated antibody has a higher binding affinity to the target compared to the activatable antibody, and
(b) providing a plurality of relationships and/or parameters characterizing reactions in the non-target compartment and/or the target compartment, wherein at least one of the reactions is a reaction that (i) converts the activatable antibody to the activated antibody which has increased affinity to binding the target compared to the activatable antibody, wherein the converting step comprises a change of conformation of at least one prodomain of the activatable antibody with respect to the AB in the activatable antibody or a cleavage of at least one prodomain away from the AB, whereby the conversion results in increased affinity to binding the target by the AB compared to the activatable antibody;
(c) determining a rate constant for the relationship or the parameter characterizing the mass transfer of activatable antibody and/or activated antibody by using first measurements of the activatable antibody and/or the activated antibody in one or more test subjects or in vitro;
(d) determining a rate constant for the relationship or the parameter characterizing at least one of the reactions by using second measurements of the activatable antibody and/or the activated antibody in the one or more test subjects or in vitro; and
(e) programming a computational system with (i) the rate constant for the relationship or the parameter characterizing the mass transfer of activatable antibody and/or activated antibody, and (ii) the rate constant for the relationship or the parameter characterizing at least one of the reactions,
whereby the computational system is programmed to (i) solve a system of expressions under a defined set of pharmacological conditions, wherein the system of expressions comprises the at least one relationship or parameter characterizing mass transfer of activatable antibody and/or activated antibody, and the plurality of relationships and/or parameters characterizing the reactions, and (ii) output of one or more predicted pharmacodynamics and/or pharmacokinetic parameter values in the subject after administration of the activatable antibody to the subject under the defined set of pharmacological conditions.

2. The method of claim 1, wherein the first measurements of the activatable antibody and/or activated antibody comprises
measurements of time-varying values of concentrations of the activatable antibody and/or the activated antibody in samples taken from the one or more test subjects who were administered one or more doses of the activatable antibody.

3. The method of claim 1 or claim 2, wherein the second measurements of the activatable antibody and/or the activated antibody comprise measurements of time-varying values of concentrations of the activatable antibody and/or activated antibody in samples taken from the one or more test subjects who were administered one or more doses of the activatable antibody.

4. The method of claim 1, 2, or 3, wherein the target compartment represents a tumor in the subject, wherein the tumor expresses the target.

5. The method of any one of claims 1 to 4, wherein the non-target compartment represents a portion of the subject that initially receives, upon administration, the activatable antibody.

6. The method of claim 5, wherein the non-target compartment represents, at least, a plasma compartment of the subject.

7. The method of any one of claims 1 to 6, wherein a first reaction of the reactions takes place in the target compartment and a second reaction of the reactions takes place in the non-target compartment.

8. The method of any one of claims 1 to 7, wherein the relationship characterizing mass transfer of the activatable antibody and/or the activated antibody is a rate expression employing concentrations of the activatable antibody and/or the activated antibody in the non-target compartment.

9. The method of any one of claims 1 to 8, further comprising providing a relationship or parameter characterizing mass transfer of the activatable antibody and/or the activated antibody between the non-target compartment of the subject and a second non-target compartment of the subject.

10. The method of claim 9, wherein the second non-target compartment represents one or more non-tumor organs or tissues in the subject.

11. The method of any one of claims 1 to 10, wherein at least one of the plurality of relationships characterizing reactions in the non-target compartment and/or the target compartment comprises cleavage of the CM of the activatable antibody.

12. The method of any one of claims 1 to 11, wherein at least one of the plurality of relationships characterizing the reactions in the non-target compartment and/or the target compartment comprises binding of the activated antibody to the target.

13. The method of any one of claims 1 to 12, wherein at least one of the plurality of relationships characterizing the reactions in the non-target compartment and/or the target compartment comprises unmasking of the AB of the activatable antibody resulting in reduced inhibition to binding the target by the AB.

14. The method of any one of claims 1 to 13, wherein at least one of the plurality of relationships characterizing the reactions in the non-target compartment and/or the target compartment comprises a relationship for a rate of cleaving the CM as a function of concentration of a protease, wherein the CM is a substrate for the protease.

15. The method of any one of claims 1 to 14, wherein the plurality of relationships and/or parameters characterizing the reactions in the non-target compartment and/or the target compartment comprises a relationship for a rate of target expression or an amount of the target.

16. The method of any one of claims 1 to 15, wherein the pharmacological conditions comprise one or more of: a dose of the activatable antibody, a frequency of dose of the activatable antibody, other medicaments administered concurrently with the activatable antibody, an activatable antibody binding affinity, an activated antibody binding affinity, a masking efficiency of the MM, a rate of cleavage of the CM, a target concentration in the target compartment, and a partition coefficient of the activatable antibody between two or more compartments.

17. The method of any one of claims 1 to 16, wherein the pharmacodynamics and pharmacokinetic parameter values comprise one or more of: a target occupancy by the activatable antibody in a target compartment; a target occupancy by the activatable antibody in a peripheral compartment; a therapeutic window; a target mediated drug disposition in a target compartment; a target mediated drug disposition in a peripheral compartment; a target mediated drug disposition in a plasma compartment; a concentration of activated antibody and/or activatable antibody in a target compartment; a concentration of activated antibody and/or activatable antibody in a plasma compartment; and a concentration of activated antibody and/or activatable antibody in a peripheral compartment.

18. The method of claim 1, wherein determining the rate constant for the relationship or the parameter characterizing the mass transfer of the activatable antibody and/or the activated antibody comprises applying an objective function to evaluate at least time-varying values of concentration of the activatable antibody and/or the activated antibody in samples taken from the one or more test subjects.

19. The method of claim 18, wherein the objective function is a log likelihood function.

20. The method of any one of claims 1 to 19, wherein determining the rate constant for the relationship or the parameter characterizing at least one of the reactions comprises applying an objective function to evaluate at least time-varying values of concentration of the activatable antibody and/or the activated antibody in samples taken from the one or more test subjects.

21. The method of claim 20, wherein the objective function is a log likelihood function.

22. The method of claim any one of claims 1 to 21, wherein the system of expressions comprises expressions for one or more zero order, first order, and/or second order rate relationships.

23. The method of any one of claims 1 to 22, wherein the system of expressions includes:
time-dependent differential equations for the activated antibody in the non-target compartment and/or time-dependent differential equations for the activatable antibody in the non-target compartment; and
time-dependent differential equations for activated antibody in the target compartment and/or time-dependent differential equations for activatable antibody in the target compartment.

24. The method of any one of claims 1 to 23, wherein the system of expressions is configured to, during execution of the quantitative systems pharmacology model, numerically solve time-dependent differential equations to provide:
a prediction of a time-dependent concentration or amount of the activated antibody in the non-target compartment and/or a time-dependent concentration or amount of the activatable antibody in the non-target compartment, and
a prediction of a time-dependent concentration or amount of the activated antibody in the target compartment and/or a time-dependent concentration or amount of the activatable antibody in the target compartment.

25. A computer program product comprising a non-transitory computer readable medium on which is provided instructions for causing a computational system to execute a quantitative systems pharmacology model for predicting pharmacodynamics and/or pharmacokinetic parameter values in a subject administered an activatable antibody, wherein the instructions comprise instructions for:
solving a system of expressions under a defined set of pharmacological conditions, wherein the system of expressions represents:
(a) at least one relationship or parameter characterizing mass transfer of activatable antibody and/or activated antibody between a non-target compartment of the subject and a target compartment of the subject, wherein the target compartment comprises a target to which at least the activated antibody binds,
wherein the activatable antibody comprises an AB and a prodomain, wherein the prodomain comprises a masking moiety (MM) and a cleavable moiety (CM), wherein the activatable antibody has a reduced binding affinity to the target compared to the AB,
wherein the activated antibody comprises an AB that includes at least one prodomain that no longer masks the AB or lacks at least one prodomain relative to the activatable antibody, wherein the activated antibody has a higher binding affinity to the target compared to the activatable antibody, and
(b) a plurality of relationships and/or parameters characterizing reactions in the non-target compartment and/or the target compartment, wherein at least one of the reactions is a reaction that (i) converts the activatable antibody to the activated antibody which has increased affinity to binding the target compared to the activatable antibody, wherein the converting step comprises a change of conformation of at least one prodomain of the activatable antibody with respect to the AB in the activatable antibody or a cleavage of at least one prodomain away from the AB, whereby the conversion results in increased affinity to binding the target by the AB compared to the activatable antibody; and
outputting one or more predicted pharmacodynamics and/or pharmacokinetic parameter values in the subject after administration of the activatable antibody to the subject under the defined set of pharmacological conditions.

26. The computer program product of claim 25, wherein a rate constant for the relationship or the parameter characterizing the mass transfer of activatable antibody and/or activated antibody was determined by using measurements of the activatable antibody and/or the activated antibody in one or more test subjects or in vitro.

27. The computer program product of claim 25 or 26, wherein the rate constant for the relationship or the parameter characterizing the mass transfer of the activatable antibody and/or the activated antibody was determined by applying an objective function to evaluate at least time-varying values of concentration of the activatable antibody and/or the activated antibody in samples taken from the one or more test subjects.

28. The computer program product of any one of claims 25 to 27, wherein the objective function is a log likelihood function.

29. The computer program product of any one of claims 25 to 28, wherein a rate constant for the relationship or the parameter characterizing at least one of the reactions of activatable antibody and/or activated antibody was determined by using measurements of the activatable antibody and/or the activated antibody in one or more test subjects or in vitro.

30. The computer program product of any one of claims 25 to 29, wherein the rate constant for the relationship or the parameter characterizing at least one of the reactions was determined by applying an objective function to evaluate at least time-varying values of concentration of the activatable antibody and/or the activated antibody in samples taken from the one or more test subjects.

31. The computer program product of claim 30, wherein the objective function is a log likelihood function.

32. The computer program product of any one of claims 25 to 31, wherein the target compartment represents a tumor in the subject, wherein the tumor expresses the target.

33. The computer program product of any one of claims 25 to 32, wherein non-target compartment represents a portion of the subject that initially receives, upon administration, the activatable antibody.

34. The computer program product of claim 33, wherein the non-target compartment represents, at least, a plasma compartment of the subject.

35. The computer program product of any one of claims 25 to 34, wherein a first reaction of the reactions takes place in the target compartment and a second reaction of the reactions takes place in the non-target compartment.

36. The computer program product of any one of claims 25 to 35, wherein the relationship characterizing mass transfer of the activatable antibody and/or the activated antibody is a rate expression employing concentrations of the activatable antibody and/or the activated antibody in the non-target compartment.

37. The computer program product of any one of claims 25 to 36, further comprising providing a relationship or parameter characterizing mass transfer of the activatable antibody and/or the activated antibody between the non-target compartment of the subject and a second non-target compartment of the subject.

38. The computer program product of claim 37, wherein the second non-target compartment represents one or more non-tumor organs or tissues in the subject.

39. The computer program product of any one of claims 25 to 38, wherein at least one of the plurality of relationships characterizing the reactions in the non-target compartment and/or the target compartment comprises cleavage of the CM of the activated antibody or the activatable antibody.

40. The computer program product of any one of claims 25 to 39, wherein at least one of the plurality of relationships characterizing the reactions in the non-target compartment and/or the target compartment comprises binding of the activated antibody to the target.

41. The computer program product of any one of claims 25 to 40, wherein at least one of the plurality of relationships characterizing the reactions in the non-target compartment and/or the target compartment comprises unmasking of the AB of the uncleaved activatable antibody resulting in reduced inhibition to binding the target by the AB.

42. The computer program product of any one of claims 25 to 41, wherein at least one of the plurality of relationships characterizing the reactions in the non-target compartment and/or the target compartment comprises a relationship for a rate of cleaving the CM as a function of concentration of a protease, wherein the CM is a substrate for the protease.

43. The computer program product of any one of claims 25 to 42, wherein the plurality of relationships and/or parameters characterizing the reactions in the non-target compartment and/or the target compartment comprises a relationship for a rate of target expression or an amount of the target.

44. The computer program product of any one of claims 25 to 43, wherein the pharmacological conditions comprise one or more of: a dose of the activatable antibody, a frequency of dose of the activatable antibody, other medicaments administered concurrently with the activatable antibody, an activatable antibody binding affinity, a activated antibody binding affinity, a masking efficiency of the MM, a rate of cleavage of the CM, a target concentration in the target compartment, and a partition coefficient of the activatable antibody between two or more compartments.

45. The computer program product of any one of claims 25 to 44, wherein the pharmacodynamics and pharmacokinetic parameter values comprise one or more of: a target occupancy by the activatable antibody in a target compartment; a target occupancy by the activatable antibody in a peripheral compartment; a therapeutic window; a target mediated drug disposition in a target compartment; a target mediated drug disposition in a peripheral compartment; a target mediated drug disposition in a plasma compartment; a concentration of cleaved and/or uncleaved activatable antibody in a target compartment; a concentration of cleaved and/or uncleaved activatable antibody in a plasma compartment; and a concentration of cleaved and/or uncleaved activatable antibody in a peripheral compartment.

46. The computer program product of any one of claims 25 to 45, wherein the rate constant for the relationship or the parameter characterizing the mass transfer of the activatable antibody and/or the activated antibody was determined by applying an objective function to evaluate at least time-varying values of concentration of the activatable antibody and/or the activated in samples taken from the one or more test subjects.

47. The computer program product of claim 46, wherein the objective function is a log likelihood function.

48. The computer program product of any one of claims 25 to 47, wherein the rate constant for the relationship or the parameter characterizing at least one of the reactions was determined by applying an objective function to evaluate at least time-varying values of concentration of the activatable antibody and/or the activated antibody in samples taken from the one or more test subjects.

49. The computer program product of claim 48, wherein the objective function is a log likelihood function.

50. The computer program product of any one of claims 25 to 49, wherein the system of expressions comprises expressions for one or more zero order, first order, and/or second order rate relationships.

51. The computer program product of any one of claims 25 to 40, wherein the system of expressions includes:
time-dependent differential equations for the activated antibody in the non-target compartment and/or time-dependent differential equations for the activatable antibody in the non-target compartment; and
time-dependent differential equations for activated antibody in the target compartment and/or time-dependent differential equations for activatable antibody in the target compartment.

52. The computer program product of any one of claims 25 to 51, wherein the system of expressions is configured to, during execution of the quantitative systems pharmacology model, numerically solve time-dependent differential equations to provide:
a prediction of a time-dependent concentration or amount of the activated antibody in the non-target compartment and/or a time-dependent concentration or amount of the activatable antibody in the non-target compartment, and
a prediction of a time-dependent concentration or amount of the activated antibody in the target compartment and/or a time-dependent concentration or amount of the activatable antibody in the target compartment.

53. A method of predicting pharmacodynamics and/or pharmacokinetic parameter values in a subject after administration of an activatable antibody, wherein the method comprises:
inputting a defined set of pharmacological conditions to a quantitative systems pharmacology model comprising instructions for solving a system of expressions under a defined set of pharmacological conditions, wherein the system of expressions represents:
(a) at least one relationship or parameter characterizing mass transfer of activatable antibody and/or activated antibody between a non-target compartment of the subject and a target compartment of the subject, wherein the target compartment comprises a target to which at least the activated antibody binds,
wherein the activatable antibody comprises an AB and a prodomain, wherein the prodomain comprises a masking moiety (MM) and a cleavable moiety (CM), wherein the activatable antibody has a reduced binding affinity to the target compared to the AB,
wherein the activated antibody comprises an AB that includes at least one prodomain that no longer masks the AB or lacks at least one prodomain relative to the activatable antibody, wherein the activated antibody has a higher binding affinity to the target compared to the activatable antibody, and
(b) a plurality of relationships and/or parameters characterizing reactions in the non-target compartment and/or the target compartment, wherein at least one of the reactions is a reaction that converts the activatable antibody to the activated antibody which has increased affinity to binding the target compared to the activatable antibody, wherein the converting step comprises a change of conformation of at least one prodomain of the activatable antibody with respect to the AB in the activatable antibody or a cleavage of at least one prodomain away from the AB, whereby the conversion results in increased affinity to binding the target by the AB compared to the activatable antibody; and
receiving from the quantitative systems pharmacology model one or more predicted pharmacodynamics and/or pharmacokinetic parameter values in the subject after administration of the activatable antibody to the subject under the defined set of pharmacological conditions.

54. The method of claim 53, further comprising using the one or more predicted pharmacodynamics and pharmacokinetic parameter values to identify or select a therapeutic activatable antibody having a selected susceptibility to cleaving the MM from the AB.

55. The method of claim 53 or 54, further comprising using the one or more predicted pharmacodynamics and pharmacokinetic parameter values to identify or select a treatment regimen for using the activatable antibody to treat a patient.

56. The method of any one of claims 53 to 55, wherein the target compartment represents a tumor in the subject, wherein the tumor expresses the target.

57. The method of any one of claims 53 to 55, wherein the non-target compartment represents a portion of the subject that initially receives, upon administration, the activatable antibody.

58. The method of claim 57, wherein the non-target compartment represents, at least, a plasma compartment of the subject.

59. The method of any one of claims 53 to 58, wherein a first reaction of the reactions takes place in the target compartment and a second reaction of the reactions takes place in the non-target compartment.

60. The method of any one of claims 53 to 59, wherein the relationship characterizing mass transfer of the activatable antibody and/or the activated antibody is a rate expression employing concentrations of the activatable antibody and/or the activated antibody in the non-target compartment.

61. The method of any one of claims 53 to 60, further comprising providing a relationship or parameter characterizing mass transfer of the activatable antibody and/or the activated antibody between the non-target compartment of the subject and a second non-target compartment of the subject.

62. The method of claim 61, wherein the second non-target compartment represents one or more non-tumor organs or tissues in the subject.

63. The method of any one of claims 53 to 62, wherein at least one of the plurality of relationships characterizing the reactions in the non-target compartment and/or the target compartment comprises cleavage of the CM of the activated antibody having one prodomain or the activatable antibody.

64. The method of any one of claims 53 to 63, wherein at least one of the plurality of relationships characterizing the reactions in the non-target compartment and/or the target compartment comprises binding of the activated antibody to the target.

65. The method of any one of claims 53 to 64, wherein at least one of the plurality of relationships characterizing the reactions in the non-target compartment and/or the target compartment comprises unmasking of the AB of the activatable antibody resulting in reduced inhibition to binding the target by the AB.

66. The method of any one of claims 53 to 65, wherein at least one of the plurality of relationships characterizing the reactions in the non-target compartment and/or the target compartment comprises a relationship for a rate of cleaving the CM as a function of concentration of a protease, wherein the CM is a substrate for the protease.

67. The method of any one of claims 53 to 66, wherein the plurality of relationships and/or parameters characterizing the reactions in the non-target compartment and/or the target compartment comprises a relationship for a rate of target expression or an amount of the target.

68. The method of any one of claims 53 to 67, wherein the system of expressions comprises expressions for one or more zero order, first order, and/or second order rate relationships.

69. The method of any one of claims 53 to 68, wherein the system of expressions includes:
time-dependent differential equations for the activated antibody in the non-target compartment and/or time-dependent differential equations for the activatable antibody in the non-target compartment; and
time-dependent differential equations for activated antibody in the target compartment and/or time-dependent differential equations for activatable antibody in the target compartment.

70. The method of any one of claims 53 to 69, wherein the system of expressions is configured to, during execution of the quantitative systems pharmacology model, numerically solve time-dependent differential equations to provide:
a prediction of a time-dependent concentration or amount of the activated antibody in the non-target compartment and/or a time-dependent concentration or amount of the activatable antibody in the non-target compartment, and
a prediction of a time-dependent concentration or amount of the activated antibody in the target compartment and/or a time-dependent concentration or amount of the activatable antibody in the target compartment.

71. The method of any one of claims 1 to 23, wherein the system of expressions is configured to, during execution of the quantitative systems pharmacology model, numerically solve time-dependent differential equations to provide:
a prediction of a dosage- and/or time-dependent concentration or amount of the activated antibody in the non-target compartment and/or a dosage- and time-dependent concentration or amount of the activatable antibody in the non-target compartment, and
a prediction of a dosage- and/or time-dependent concentration or amount of the activated antibody in the target compartment and/or a dosage- and time-dependent concentration or amount of the activatable antibody in the target compartment.

72. The computer program product of any one of claims 25 to 51, wherein the system of expressions is configured to, during execution of the quantitative systems pharmacology model, numerically solve time-dependent differential equations to provide:
a prediction of a dosage- and time-dependent concentration or amount of the activated antibody in the non-target compartment and/or a dosage- and/or time-dependent concentration or amount of the activatable antibody in the non-target compartment, and
a prediction of a dosage- and time-dependent concentration or amount of the activated antibody in the target compartment and/or a dosage- and/or time-dependent concentration or amount of the activatable antibody in the target compartment.

73. The method of any one of claims 53 to 69, wherein the system of expressions is configured to, during execution of the quantitative systems pharmacology model, numerically solve time-dependent differential equations to provide:
a prediction of a dosage- and/or time-dependent concentration or amount of the activated antibody in the non-target compartment and/or a dosage- and/or time-dependent concentration or amount of the activatable antibody in the non-target compartment, and
a prediction of a dosage- and time-dependent concentration or amount of the activated antibody in the target compartment and/or a dosage- and/or time-dependent concentration or amount of the activatable antibody in the target compartment.

74. The method of any one of claims 1 to 23, wherein the system of expressions is configured to, during execution of the quantitative systems pharmacology model, numerically solve time-dependent differential equations to provide:
a prediction of a dosage- and/or time-dependent receptor of target occupancy by the activated antibody in the non-target compartment and/or a dosage- and/or time-dependent concentration or amount of the activatable antibody in the non-target compartment, and
a prediction of a dosage- and/or time-dependent receptor of target occupancy by the activated antibody in the target compartment and/or a dosage- and/or time-dependent receptor of target occupancy by the activatable antibody in the target compartment.

75. The computer program product of any one of claims 25 to 51, wherein the system of expressions is configured to, during execution of the quantitative systems pharmacology model, numerically solve time-dependent differential equations to provide:
a prediction of a dosage- and/or time-dependent receptor of target occupancy by the activated antibody in the non-target compartment and/or a dosage- and/or time-dependent concentration or amount of the activatable antibody in the non-target compartment, and
a prediction of a dosage- and/or time-dependent receptor of target occupancy by the activated antibody in the target compartment and/or a dosage- and/or time-dependent receptor of target occupancy by the activatable antibody in the target compartment.

76. The method of any one of claims 53 to 69, wherein the system of expressions is configured to, during execution of the quantitative systems pharmacology model, numerically solve time-dependent differential equations to provide:
a prediction of a dosage- and/or time-dependent receptor of target occupancy by the activated antibody in the non-target compartment and/or a dosage- and/or time-dependent concentration or amount of the activatable antibody in the non-target compartment, and a prediction of a dosage- and/or time-dependent receptor of target occupancy by the activated antibody in the target compartment and/or a dosage- and/or time-dependent receptor occupancy by the activatable antibody in the target compartment.

77. The method of any one of claims 1 to 23, wherein the system of expressions is configured to, during execution of the quantitative systems pharmacology model, numerically solve time-dependent differential equations to provide:
a prediction of a dosage- and/or time-dependent receptor of target occupancy by the activated antibody in the non-target compartment and/or a dosage- and/or time-dependent concentration or amount of the activatable antibody in the non-target compartment,
a prediction of a dosage- and/or time-dependent receptor of target occupancy by the activated antibody in the target compartment and/or a dosage- and/or time-dependent receptor of target occupancy by the activatable antibody in the target compartment, and
a prediction of a biologically effective dose based on the dosage that results in a given target occupancy by the activated and activatable antibody in the target compartment.

78. The computer program product of any one of claims 25 to 51, wherein the system of expressions is configured to, during execution of the quantitative systems pharmacology model, numerically solve time-dependent differential equations to provide:
a prediction of a dosage- and/or time-dependent receptor of target occupancy by the activated antibody in the non-target compartment and/or a dosage- and/or time-dependent concentration or amount of the activatable antibody in the non-target compartment, and
a prediction of a biologically effective dose based on the dosage that results in a given target occupancy by the activated and activatable antibody in the target compartment.

79. The method of any one of claims 53 to 69, wherein the system of expressions is configured to, during execution of the quantitative systems pharmacology model, numerically solve time-dependent differential equations to provide:
a prediction of a dosage- and/or time-dependent receptor of target occupancy by the activated antibody in the non-target compartment and/or a dosage- and/or time-dependent concentration or amount of the activatable antibody in the non-target compartment, and
a prediction of a biologically effective dose based on the dosage that results in a given target occupancy by the activated and activatable antibody in the target compartment.
